(19) United States
(12) Patent Application Publication
(10) Pub. No.: US 2016/0009725 A1
(43) Pub. Date: Jan. 14, 2016

(54) PYRROLO- AND PYRAZOLO-TRIAZOLODIAZEPINES AS BET-PROTEIN INHIBITORS FOR TREATING HYPERPROLIFERATIVE DISEASES

(71) Applicant: BAYER PHARMA AKTIENGESELLSCHAFT, Berlin (DE)

(72) Inventors: Norbert SCHMEES, Berlin (DE); Bernd BUCHMANN, Hohen Neuendorf (DE); Bernard HAENDLER, Berlin (DE); Roland NEUHAUS, Berlin (DE); Pascale LEJEUNE, Berlin (DE); Martin KRÜGER, Berlin (DE); Amaury Ernesto FERNANDEZ-MONTALVAN, Berlin (DE); Hermann KÜNZER, (US)

(73) Assignee: BAYER PHARMA AKTIENGESELLSCHAFT, Berlin (DE)

(21) Appl. No.: 14/770,000

(22) PCT Filed: Feb. 17, 2014
(86) PCT No.: PCT/EP2014/052988

§ 371 Date: Aug. 24, 2015

(30) Foreign Application Priority Data
Feb. 22, 2013 (DE) 10 2013 202 993.4

(51) Int. Cl. C07D 487/14; C07D 498/08; C07D 495/10; C07D 491/08; C07D 491/107; A61K 31/551

(52) U.S. Cl. CPC C07D 487/14; C07D 491/107; A61K 31/551; C07D 495/10; C07D 491/08; C07D 498/08

(57) ABSTRACT

What is described are BET protein-inhibitory, especially BRD2-, BRD3- and BRD4-inhibitory, bicyclo- and spino-substituted pyrrolo- and pyrazolodiazepines of the general formula I

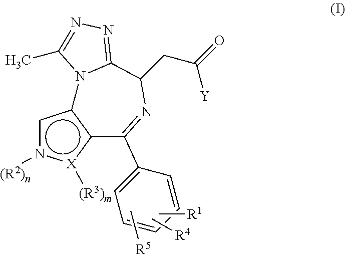

in which X, Y, n, m, R¹, R², R³, R⁴ and R⁵ are each as defined in the description, to intermediates for preparation of the inventive compounds, to pharmaceutical compositions comprising the inventive compounds, and to the prophylactic and therapeutic use thereof for hyperproliferative disorders, especially for tumour disorders. Also described is the use of the compounds according to the invention as BET protein inhibitors for benign hyperplasias, for atherosclerotic disorders, for sepsis, for autoimmune disorders, for vascular disorders, for viral infections, for neurodegenerative disorders, for inflammatory disorders, for atherosclerotic disorders and for male fertility control.

The present invention relates to BET protein-inhibitory, especially BRD2-, BRD3- and BRD4-inhibitory, bicyclo- and spino-substituted pyrrolo- and pyrazolodiazepines, to intermediates for preparation of the compounds according to the invention, to pharmaceutical compositions comprising the compounds according to the invention, and to the prophylactic and therapeutic use thereof for hyperproliferative disorders, especially for neoplastic disorders. The present invention further relates to the use of BET protein inhibitors for benign hyperplasias, for atherosclerotic disorders, for sepsis, for autoimmune disorders, for vascular disorders, for viral infections, for neurodegenerative disorders, for inflammatory disorders, for atherosclerotic disorders and for male fertility control.

The human BET family (bromo domain and extra C-terminal domain family) has four members (BRD2, BRD3, BRD4 and BRDT) containing two related bromo domains and one extraterminal domain (Wu and Chiang, J. Biol. Chem., 2007, 282:13141-13145). The bromo domains are protein regions which recognize acetylated lysine residues. Such acetylated lysines are often found at the N-terminal end of histones (e.g. histone H3 or histone H4), and they are features of an open chromatin structure and active gene transcription (Kuo and Allis, Bioessays, 1998, 20:615-626). The different acetylation patterns recognized by BET proteins in histones were investigated in depth (Umehara et al., J. Biol. Chem., 2010, 285:7610-7618; Filippakopoulos et al., Cell, 2012, 149:214-231). In addition, bromo domains can recognize further acetylated proteins. For example, BRD4 binds to RelA, which leads to stimulation of NF-B and transcriptional activity of inflammatory genes (Huang et al., Mol. Cell. Biol., 2009, 29:1375-1387; Zhang et al., J. Biol. Chem., 2012, doi/10.1074/jbc.M112.359505). The extraterminal domain of BRD2, BRD3 and BRD4 interacts with several proteins involved in chromatin modulation and the regulation of gene expression (Rahman et al., Mol. Cell. Biol., 2011, 31:2641-2652).

In mechanistic terms, BET proteins play an important role in cell growth and in the cell cycle. They are associated with mitotic chromosomes, suggesting a function in epigenetic memory (Dey et al., Mol. Biol. Cell, 2009, 20:4899-4909; Yang et al., Mol. Cell. Biol., 2008, 28:967-976). BRD4 is important for post-mitotic reactivation of gene transcription (Zhao et al., Nat. Cell. Biol., 2011, 13:1295-1304). It has been shown that BRD4 is essential for transcription elongation and for the recruitment of the elongation complex P-TEFb consisting of CDK9 and cyclin T1, which leads to activation of RNA polymerase II (Yang et al., Mol. Cell, 2005, 19:535-545; Schröder et al., J. Biol. Chem., 2012, 287:1090-1099). Consequently, the expression of genes involved in cell proliferation is stimulated, for example of c-Myc and aurora B (You et al., Mol. Cell. Biol., 2009, 29:5094-5103; Zuber et al., Nature, 2011, 478:524-528). BRD2 and BRD3 bind to transcribed genes in hyperacetylated chromatin regions and promote transcription by RNA polymerase II (LeRoy et al., Mol. Cell, 2008, 30:51-60).

Knock-down of BRD4 or the inhibition of the interaction with acetylated histones in various cell lines leads to G1 arrest and to cell death apoptosis (Mochizuki et al., J. Biol. Chem., 2008, 283:9040-9048; Mertz et al., Proc. Natl. Acad. Sci. USA, 2011, 108:16669-16674). It has also been shown that BRD4 binds to promoter regions of several genes which are activated in the G1 phase, for example cyclin D1 and D2 (Mochizuki et al., J. Biol. Chem., 2008, 283:9040-9048). In addition, inhibition of the expression of c-Myc, an essential factor in cell proliferation, after BRD4 inhibition has been demonstrated (Dawson et al., Nature, 2011, 478:529-533; Delmore et al., Cell, 2011, 146:1-14; Mertz et al., Proc. Natl. Acad. Sci. USA, 2011, 108:16669-16674).

BRD2 and BRD4 knockout mice die early in embryogenesis (Gyuris et al., Biochim Biophys. Acta, 2009, 1789:413-421; Houzelstein et al., Mol. Cell. Biol., 2002, 22:3794-3802). Heterozygotic BRD4 mice have various growth defects attributable to reduced cell proliferation (Houzelstein et al., Mol. Cell. Biol., 2002, 22:3794-3802).

BET proteins play an important role in various tumour types. Fusion between the BET proteins BRD3 or BRD4 and NUT, a protein which is normally expressed only in the testes, leads to an aggressive form of squamous cell carcinoma, called NUT midline carcinoma (French, Cancer Genet. Cytogenet., 2010, 203:16-20). The fusion protein prevents cell differentiation and promotes proliferation (Yan et al., J. Biol. Chem., 2011, 286:27663-27675). The growth of in vivo models derived therefrom is inhibited by a BRD4 inhibitor (Filippakopoulos et al., Nature, 2010, 468:1067-1073). Screening for therapeutic targets in an acute myeloid leukaemia cell line (AML) showed that BRD4 plays an important role in this tumour (Zuber et al., Nature, 2011, doi:10.1038). Reduction in BRD4 expression leads to a selective arrest of the cell cycle and to apoptosis. Treatment with a BRD4 inhibitor prevents the proliferation of an AML xenograft in vivo. Amplification of the DNA region containing the BRD4 gene was detected in primary breast tumours (Kadota et al., Cancer Res, 2009, 69:7357-7365). For BRD2 too, there are data relating to a role in tumours. A transgenic mouse which overexpresses BRD2 selectively in B cells develops B cell lymphoma and leukaemia (Greenwall et al., Blood, 2005, 103:1475-1484).

BET proteins are also involved in viral infections. BRD4 binds to the E2 protein of various papillomaviruses and is important for the survival of the viruses in latently infected cells (Wu et al., Genes Dev., 2006, 20:2383-2396; Vosa et al., J. Virol., 2012, 86:348-357). The herpes virus, which is responsible for Kaposi's sarcoma, also interacts with various BET proteins, which is important for disease survival (Viejo-Borbolla et al., J. Virol., 2005, 79:13618-13629; You et al., J. Virol., 2006, 80:8909-8919). Through binding to P-TEFb, BRD4 also plays an important role in the replication of HIV (Bisgrove et al., Proc. Natl. Acad. Sci. USA, 2007, 104:13690-13695).

BET proteins are additionally involved in inflammation processes. BRD2-hypomorphic mice show reduced inflammation in adipose tissue (Wang et al., Biochem. J., 2009, 425:71-83). Infiltration of macrophages in white adipose tissue is also reduced in BRD2-deficient mice (Wang et al., Biochem. J., 2009, 425:71-83). It has also been shown that BRD4 regulates a number of genes involved in inflammation. In LPS-stimulated macrophages, a BRD4 inhibitor prevents the expression of inflammatory genes, for example IL-1 or IL-6 (Nicodeme et al., Nature, 2010, 468:1119-1123). BET proteins also regulate the expression of the ApoA1 gene which plays an important role in atherosclerosis and inflammatory processes (Chung et al., J. Med. Chem, 2011, 54:3827-3838). Apolipoprotein A1 (ApoA1) is a major component of high density lipoproteins (HDL), and increased expression of ApoA1 leads to elevated blood cholesterol values (Degoma and Rader, Nat. Rev. Cardiol., 2011, 8:266-277). Elevated HDL values are associated with a reduced risk of atherosclerosis (Chapman et al., Eur. Heart J., 2011, 32:1345-1361).

### PRIOR ART

The nomenclature employed in the assessment of the structural prior art is illustrated by the following figure:

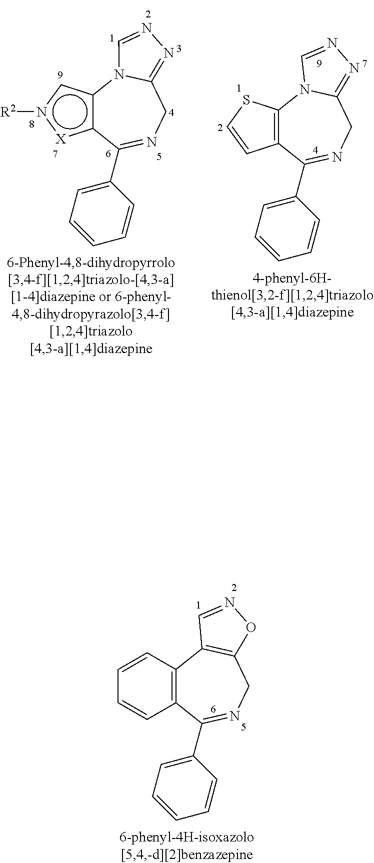

Based on the chemical structure, some types of BRD4 inhibitors have been described to date (Chun-Wa Chung et al., Progress in Medicinal Chemistry 2012, 51, 1-55).

The first published BRD4 inhibitors are phenylthienotriazolo-1,4-diazepines (4-phenyl-6H-thieno[3,2-f][1,2,4]triazolo[4,3-a][1,4]diazepines) as described in WO2009/084693 (Mitsubishi Tanabe Pharma Corporation) and as compound JQ1 in WO2011/143669 (Dana Farber Cancer Institute). Replacement of the thieno moiety by a benzo moiety also leads to active inhibitors (J. Med. Chem. 2011, 54, 3827-3838; E. Nicodeme et al., Nature 2010, 468, 1119). These and one further publication show that the pyrazole unit fused to the 1,4-benzodiazepine or thieno-1,4-diazepine ring system is actively involved in binding of the target protein BRD4 (P. Filippakopoulos et al., Nature 2010, 468, 1067). Further 4-phenyl-6H-thieno[3,2-f][1,2,4]triazolo[4,3-a][1,4]diazepines and related compounds having alternative rings as a fusion partner rather than the benzo unit are addressed generically or described directly in WO2012/075456 (Constellation Pharmaceuticals). WO2012/075383 (Constellation Pharmaceuticals) describes 6-substituted 4H-isoxazolo[5,4-d][2]benzazepines and 4H-isoxazolo[3,4-d][2]benzazepines, including compounds which have optionally substituted phenyl at position 6 as BRD4 inhibitors, and also analogues with alternative heterocyclic fusion partners rather than the benzo unit, for example thieno- or pyridoazepines.

WO2013/184876 and WO2013/184878 (Constellation Pharmaceuticals) describe further benzoisoxazoloazepine derivatives as inhibitors of proteins comprising bromo domains.

Another structural class of BRD4 inhibitors described is that of 7-isoxazoloquinolines and related quinolone derivatives (WO2011/054843, Bioorganic & Medicinal Chemistry Letters 22 (2012) 2963-2967, GlaxoSmithKline). Pyridinones and pyridazinones (WO 2013/185284, WO 2013/188381; Abbott Laboratories) and also isoindolones (WO 2013/155695, WO 2013/158952; Abbott Laboratories) have been described as inhibitors of binding of the bromo domains of the BET proteins to proteins comprising N-acetylated lysine residues.

WO94/26718/EP0703222A1 (Yoshitomi Pharmaceutical Industries) describes substituted 3-amino-2,3-dihydro-1H-1-benzazepin-2-ones or the corresponding 2-thiones and analogues in which the benzo unit has been replaced by alternative monocyclic systems, and in which the 2-ketone or the 2-thione together with the substituted nitrogen atom in the azepine ring may form a heterocycle, as CCK and gastrin antagonists for the treatment of CNS disorders, such as states of anxiety and depression, and of pancreatic disorders and of gastrointestinal ulcers. Ligands of the gastrin and the cholecystokinin receptor are described in WO2006/051312 (James Black Foundation). They also include substituted 3,5-dihydro-4H-2,3-benzodiazepin-4-ones which differ from the compounds according to the invention mainly by the obligatory oxo group in position 4 and by an obligatory carbonyl group-containing alkyl chain in position 5. Finally, substituted 3,5-dihydro-4H-2,3-benzodiazepin-4-ones are also described as AMPA antagonists in WO97/34878 (Cocensys Inc.). The generic claim is very wide with respect to the possible substitution patterns at the benzodiazepine skeleton; however, the working examples are limited to a very narrow range.

EP102602 furthermore describes 6-aryldiazepinones having a fused pyrrole ring which are used as spasmolytics and for anxiety. These may carry side chains in position 4 which are attached via an oxygen or nitrogen atom. Attachment via a carbon atom has hitherto not been described. DE3435973 describes 6-aryltriazolodiazepines which carry a fused pyrrole ring having a nitrogen in position 2. The compounds are used for treating pathological states and diseases where acetyl glyceryl ether phosphorylcholine (PAF) is involved. However, these compounds do not have a side chain in position 4. Only substitution by a methyl group has been described at a diazepinone system having a fused pyrazole (J. Med. Chem. 24, (1981), p 982ff, DeWald et al.).

Furthermore, fusion of pyrazole to the nitrogen atoms in positions 2 and 3 is described in U.S. Pat. No. 3,657,271. However, these compounds do not carry a further fused triazole ring and no side chain in position 4 either. The compounds demonstrate anti-inflammatory activity.

It would therefore be desirable to provide novel compounds having improved prophylactic and therapeutic properties.

Accordingly, it is an object of the present invention to provide compounds and pharmaceutical compositions comprising these compounds used for prophylactic and therapeutic applications for hyperproliferative disorders, in particular for tumour disorders, and as BET protein inhibitors for viral infections, for benign hyperplasias, for neurodegenerative disorders, for inflammatory disorders, for atherosclerotic disorders, for autoimmune disorders, for vascular disorders, for sepsis and for male fertility control.

The compounds according to the invention are novel pyrrolo- and pyrazolodiazepines having a bicyclo or spiro group at the diazepine skeleton which, surprisingly, have BET protein-inhibitory, especially BRD2-, BRD3- and BRD4-inhibitory, activity, and which inhibit interaction between BRD4 inhibitors and an acetylated histone H4 peptide and inhibit the growth of cancer cells.

From the prior art described above, there was no reason to modify the structures of the prior art such that structures suitable for the prophylaxis and therapy of tumour disorders are obtained.

Surprisingly, the compounds according to the invention inhibit the interaction between BRD4 and an acetylated histone H4 peptide and inhibit the growth of cancer cells. Accordingly, they provide novel structures for the therapy of human and animal disorders, in particular of cancers.

It has now been found that compounds of the general formula I

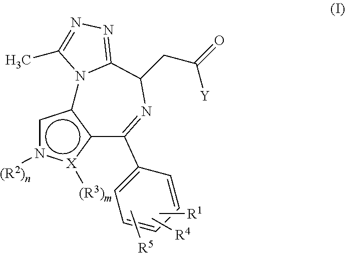

where
X represents a carbon or nitrogen atom,
n and m independently of one another represent 0 or 1,
R¹, R⁴ and R⁵ identical or different from one another represent hydrogen, hydroxy, cyano, nitro, amino, aminocarbonyl-, halogen or represent C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, aminocarbonyl-, C₁-C₆-alkylcarbonyl-, C₁-C₆-alkylaminocarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms, where the monocyclic heterocyclyl and heteroaryl radicals for their part are optionally monosubstituted by C₁-C₃-alkyl,
   or
   represents C₃-C₁₀-cycloalkyl- which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
   or
   represents monocyclic heteroaryl- having 5 or 6 ring atoms which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylsulphonyl-, C₁-C₆-alkylamino-C₁-C₆-alkyl, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represents monocyclic heterocyclyl- having 3 to 8 ring atoms which is optionally mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkylcarbonyl-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represents phenyl- which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylcarbonyl-, C₁-C₆-alkylaminosulphonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R³ represents hydrogen, hydroxy, cyano, nitro, amino, aminocarbonyl-, halogen or represents C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl—which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a carbon atom,
   or
R³ represents hydrogen or represents C₁-C₆-alkyl-, C₁-C₆-alkoxy-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a nitrogen atom,
   or
R² and R³ together with the ring atoms N and X may form a further heteroaromatic or heterocyclic ring having 5 to 7 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
   and their polymorphs, enantiomers, diastereomers, racemates, tautomers, solvates, physiologically acceptable salts and solvates of these salts are particularly suitable for a large number of prophylactic and therapeutic applications, in particular for hyperproliferative disorders, for tumour disorders and as BET protein inhibitors, for benign hyperplasias, atherosclerotic disorders, sepsis, autoimmune disorders, vascular disorders, viral infections, for neurodegenerative disorders, for inflammatory disorders and for male fertility control.

Of particular interest are those compounds of the general formula I in which
X represents a carbon or nitrogen atom,
n and m independently of one another represent 0 or 1,
R¹, R⁴ and R⁵ identical or different from one another represent hydrogen, hydroxy, cyano, nitro, amino, aminocarbonyl-, halogen or represent C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms, where the monocyclic heterocyclyl and heteroaryl radicals for their part are optionally monosubstituted by C₁-C₃-alkyl,
   or
   represents C₃-C₁₀-cycloalkyl- which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
   or
   represent monocyclic heterocyclyl- having 3 to 8 ring atoms which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms, or represents monocyclic heteroaryl- having 5 or 6 ring atoms which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylsulphonyl-, C₁-C₆-alkylamino-C₁-C₆-alkyl, halo-C₁-C₆-alkyl-, halo-C₁-C₆-alkoxy-, C₃-C₁₀-cycloalkyl- and a monocyclic heterocyclyl radical having 3 to 8 ring atoms,
   or
   represents phenyl- which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a carbon atom,
   or
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a nitrogen atom,
   or
R² and R³ together with the ring atoms N and X may form a further heteroaromatic or heterocyclic ring having 5 to 7 ring atoms which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
   and their polymorphs, enantiomers, diastereomers, racemates, tautomers, solvates, physiologically acceptable salts and solvates of these salts.

Particularly interesting are those compounds of the general formula I in which
X represents a carbon or nitrogen atom,
n and m independently of one another represent 0 or 1,
R¹, R⁴ and R⁵ identical or different from one another represent hydrogen, hydroxy, cyano, aminocarbonyl-, halogen or represent C₁-C₆-alkyl-, C₁-C₆-alkoxy, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy and carboxy,
   or
   represents C₃-C₁₀-cycloalkyl- which is optionally mono- or polysubstituted by identical or different substituents from the group consisting of halogen, C₁-C₆-alkyl- and C₁-C₆-alkoxy-,
   or
   represents phenyl- which is optionally mono- or polysubstituted by identical or different substituents from the group consisting of halogen, hydroxy, cyano, carboxy, C₁-C₆-alkyl-, C₁-C₆-alkoxy-, halo-C₁-C₆-alkyl-, halo-C₁-C₆-alkoxy-, C₃-C₁₀-cycloalkyl- and a monocyclic heterocyclyl radical having 3 to 8 ring atoms,
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a carbon atom, or
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a nitrogen atom,
   or
R² and R³ together with the ring atoms N and X may form a further heteroaromatic or heterocyclic ring having 5 to 7 ring atoms which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, hydroxy, amino, oxo, C₁-C₆-alkyl-, C₁-C₆-alkoxy-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-, and
R⁹ represents C₁-C₆-alkyl-,
   and their polymorphs, enantiomers, diastereomers, racemates, tautomers, solvates, physiologically acceptable salts and solvates of these salts.

Very particularly interesting are those compounds of the general formula I in which
X represents a carbon or nitrogen atom,
n and m independently of one another represent 0 or 1,
R¹, R⁴ and R⁵ identical or different from one another represent hydrogen, cyano, halogen or represent C₁-C₆-alkyl- or C₁-C₆-alkoxy- which is optionally mono- or polysubstituted by halogen,
R² represents hydrogen or represents C₁-C₆-alkyl- which is optionally mono- or polysubstituted by halogen,
   or
   represent phenyl which is optionally mono- or polysubstituted by identical or different substituents from the group consisting of halogen, cyano, C₁-C₆-alkyl-, C₁-C₆-alkoxy-, halo-C₁-C₆-alkyl- and halo-C₁-C₆-alkoxy-,
R³ represents hydrogen or C₁-C₆-alkyl- which is optionally mono- or polysubstituted by identical or different substituents from the group consisting of halogen, amino, hydroxy, C₁-C₆-alkoxy- and C₁-C₆-alkoxy-C₁-C₆-alkyl-, if X represents a carbon atom,
   or
R³ represents hydrogen or C₁-C₆-alkyl- which is optionally mono- or polysubstituted by identical or different substituents from the group consisting of halogen and C₁-C₆-alkoxy-, if X represents a nitrogen atom, or
R² and R³ together with the ring atoms N and X may form a further heteroaromatic or heterocyclic ring having 5 to 7 ring atoms which is optionally mono- or polysubstituted by identical or different substituents from the group consisting of halogen, amino, hydroxy, cyano, C₁-C₆-alkyl-, C₁-C₆-alkoxy-, halo-C₁-C₆-alkyl- and halo-C₁-C₆-alkoxy-,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, oxo, hydroxy, C₁-C₃-alkyl-, C₁-C₃-alkoxy-, halo-C₁-C₃-alkyl-, halo-C₁-C₃-alkoxy-, C₃-C₇-cycloalkyl-, phenyl-, halophenyl-, phenyl-C₁-C₃-alkyl-, phenyl-C₁-C₃-alkoxy-, —C(═O)—NR⁶R⁷, —C(═O)—R⁸, —S(═O)₂—NR⁶R⁷, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₃-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
   and their polymorphs, enantiomers, diastereomers, racemates, tautomers, solvates, physiologically acceptable salts and solvates of these salts.

Preference is given to those compounds of the general formula I in which
X represents a carbon or nitrogen atom,
n and m independently of one another represent 0 or 1,
R¹, R⁴ and R⁵ identical or different from one another represent hydrogen or halogen,
R² represents hydrogen or represents C₁-C₃-alkyl-,
R³ represents hydrogen or C₁-C₃-alkyl-, if X represents a carbon atom,
   or
R³ represents hydrogen or represents C₁-C₃-alkyl-, if X represents a nitrogen atom,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, oxo, cyano, hydroxy, C₁-C₃-alkyl-, C₁-C₃-alkoxy-, halo-C₁-C₃-alkyl-, halo-C₁-C₃-alkoxy-, phenyl-, halophenyl-, phenyl-C₁-C₃-alkyl-, phenyl-C₁-C₃-alkoxy-, —C(═O)—NR⁶R⁷, —C(═O)—R⁸, —S(═O)₂—R⁹ and —NH—S(═O)₂—R⁹,
R⁶ and R⁷ independently of one another represent hydrogen or C₁-C₃-alkyl,
R⁸ represents hydroxy, C₁-C₆-alkyl-, C₁-C₃-alkoxy-, halo-C₁-C₃-alkyl-, phenyl- or monocyclic heterocyclyl- having 3 to 8 ring atoms where phenyl- and heterocyclyl- are optionally mono- or disubstituted by halogen, C₁-C₃-alkoxy- or C₁-C₃-alkyl-,
R⁹ represents C₁-C₃-alkyl-,
   and their polymorphs, enantiomers, diastereomers, racemates, tautomers, solvates, physiologically acceptable salts and solvates of these salts.

Even more preference is given to those compounds of the general formula I in which
X represents a carbon or nitrogen atom,
n and m independently of one another represent 0 or 1,
R¹ represents hydrogen or halogen,
R² represents hydrogen or represents C₁-C₃-alkyl-,
R³ represents hydrogen or C₁-C₃-alkyl-, if X represents a carbon atom,
   or
R³ represents hydrogen or represents C₁-C₃-alkyl-, if X represents a nitrogen atom,
R⁴ and R⁵ represent hydrogen,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of oxo, fluoro, chloro, bromo, cyano, hydroxy, methyl, ethyl, methoxy, ethoxy, trifluoromethyl, benzyl, phenyl and —C(═O)—R⁸,
R⁸ represents hydroxy, C₁-C₆-alkyl- or halo-C₁-C₃-alkyl-,
   and their polymorphs, enantiomers, diastereomers, racemates, tautomers, solvates, physiologically acceptable salts and solvates of these salts.

Particular preference is given to those compounds of the general formula I in which
X represents a carbon or nitrogen atom,
n and m independently of one another represent 0 or 1,
R¹ represents hydrogen, fluorine, chlorine or bromine,
R² represents hydrogen or represents methyl,
R³ represents hydrogen or methyl, if X represents a carbon atom,
   or
R³ represents hydrogen or methyl, if X represents a nitrogen atom,
R⁴ and R⁵ represent hydrogen,
Y represents a spirocycloalkyl- or heterospirocycloalkyl radical of 7 to 11 ring atoms, a bridged cycloalkyl radical or a bridged heterocycloalkyl radical of 7 to 8 ring atoms, where the radicals mentioned here are optionally mono- or polysubstituted by identical or different substituents from the group consisting of oxo, fluoro, chloro, bromo, cyano, hydroxy, methyl, ethyl, methoxy, ethoxy, trifluoromethyl, benzyl, phenyl and —C(═O)—R⁸,
R⁸ represents hydroxy, C₁-C₆-alkyl- or halo-C₁-C₃-alkyl-,
   and their polymorphs, enantiomers, diastereomers, racemates, tautomers, solvates, physiologically acceptable salts and solvates of these salts.

Particular preference is given to those compounds of the general formula I in which
X represents a carbon or nitrogen atom,
n and m independently of one another represent 0 or 1,
R¹ represents hydrogen or chlorine,
R² represents hydrogen or represents methyl,
R³ represents methyl, if X represents a carbon atom,
   or
R³ represents hydrogen or methyl, if X represents a nitrogen atom,
R⁴ and R⁵ represent hydrogen,
Y represents a radical

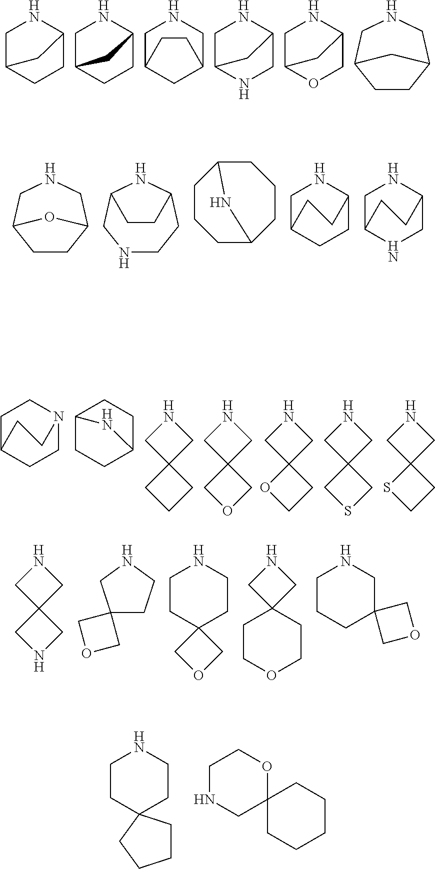

where the radicals mentioned here are optionally mono- or disubstituted by identical or different substituents from the group consisting of oxo, fluorine, chlorine, bromine, cyano, hydroxy, methyl, ethyl, methoxy, ethoxy, benzyl, phenyl and —C(═O)—R⁸,
R⁸ represents hydroxy, C₁-C₆-alkyl- or halo-C₁-C₃-alkyl-,
   and their polymorphs, enantiomers, diastereomers, racemates, tautomers, solvates, physiologically acceptable salts and solvates of these salts.

Very particular preference is given to those compounds of the general formula I in which
X represents a carbon or nitrogen atom,
n and m independently of one another represent 0 or 1,
R¹ represents hydrogen or chlorine,
R² represents hydrogen or represents methyl,
R³ represents methyl, if X represents a carbon atom,
   or
R³ represents hydrogen or methyl, if X represents a nitrogen atom,
R⁴ and R⁵ represent hydrogen,
Y represents a radical

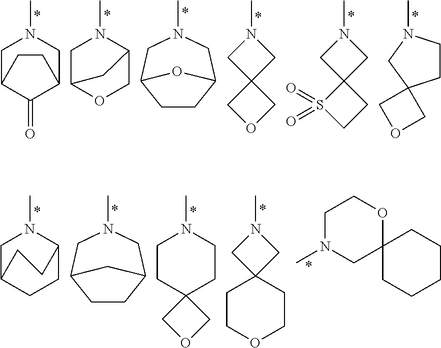

where “*” denotes the point of attachment to the remainder of the molecule,
   and their polymorphs, enantiomers, diastereomers, racemates, tautomers, solvates, physiologically acceptable salts and solvates of these salts.

Extraordinary preference is given to the following compounds:
(−)-2-[(4S)-6-(4-chlorophenyl)-1,7,8-trimethyl-4,8-dihydropyrrolo[3,4-f][1,2,4]triazolo-[4,3-a][1,4]diazepin-4-yl]-1-(2-oxa-6-azaspiro[3.3]hept-6-yl)ethan-1-one;
2-(1,7,8-trimethyl-6-phenyl-4,8-dihydropyrrolo[3,4-f][1,2,4]triazolo[4,3-a][1,4]diazepin-4-yl)-1-(2-oxa-6-azaspiro[3.3]hept-6-yl)ethan-1-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1,1-dioxo-1λ6-1-thia-6-azaspiro[3.3]hept-6-yl)ethan-1-one;
2-[(4S)-6-(4-chlorophenyl)-1,8-dimethyl-4,8-dihydropyrazolo[3,4-f][1,2,4]triazolo[4,3-a][1,4]diazepin-4-yl]-1-(2-oxa-6-azaspiro[3.3]hept-6-yl)ethan-1-one;
2-[(4S)-6-(4-chlorophenyl)-1,7-dimethyl-4,7-dihydropyrazolo[3,4-f][1,2,4]triazolo[4,3-a][1,4]diazepin-4-yl]-1-(2-oxa-6-azaspiro[3.3]hept-6-yl)ethan-1-one;
(−)-8-{2-[(4S)-6-(4-chlorophenyl)-1,7,8-trimethyl-4,8-dihydropyrrolo[3,4-f][1,2,4]triazolo[4,3-a][1,4]diazepin-4-yl]acetyl}-8-azabicyclo[3.2.1]octan-3-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-{(1S,4S)-2-oxa-5-azabicyclo[2.2.1]hept-5-yl}-ethan-1-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(8-oxa-3-azabicyclo[3.2.1]oct-3-yl)ethan-1-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1-oxa-4-azaspiro[5.5]undec-4-yl)ethanone;
(−)-1-(3-azabicyclo[3.2.1]oct-3-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(7-oxa-2-azaspiro[3.5]non-2-yl)ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-7-azaspiro[3.5]non-7-yl)ethanone;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-6-azaspiro[3.4]oct-6-yl)ethanone;
(−)-1-(2-azabicyclo[2.2.2]oct-2-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone and
2-[6-(4-chlorophenyl)-1,8-dimethyl-4,8-dihydropyrazolo[3,4-f][1,2,4]triazolo[4,3-a][1,4]diazepin-4-yl]-1-(1,1-dioxido-1-thia-6-azaspiro[3.3]hept-6-yl)ethanone.

In the general formula I, X may represent a carbon or nitrogen atom.

In the general formula I, m and n independently of one another may represent 0 or 1.

In the general formula I, R¹, R⁴ and R⁵ may be identical or different from one another and represent hydrogen, hydroxy, cyano, nitro, amino, aminocarbonyl-, halogen or represent C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-.

In the general formula I, of very particular interest are compounds in which R¹, R⁴ and R⁵ are identical or different from one another, represent hydrogen, cyano, halogen or represent C₁-C₆-alkyl- or C₁-C₆-alkoxy- which is optionally mono- or polysubstituted by halogen.

In the general formula I, preference is given to those compounds in which R¹, R⁴ and R⁵, identical or different from one another, represent hydrogen or halogen.

In the general formula I, even more preference is given to those compounds in which R¹ represents hydrogen or halogen and R⁴ and R⁵ represents hydrogen.

In the general formula I, special preference is given to those compounds in which R¹ represents hydrogen or chlorine and R⁴ and R⁵ represents hydrogen.

In the general formula I, of special interest are compounds in which R² represents hydrogen or represents C₁-C₆-alkyl-, C₁-C₆-alkylcarbonyl-, phenylsulphonyl- or C₁-C₆-alkylsulphonyl-, where the radicals mentioned may optionally be mono- or polysubstituted by identical or different substituents from the group consisting of halogen, amino, hydroxy and carboxy,

or
represents C₃-C₁₀-cycloalkyl- which may optionally be mono- or polysubstituted by identical or different substituents from the group consisting of halogen, C₁-C₆-alkyl- or C₁-C₆-alkoxy-,
or
represents phenyl- which is optionally mono- or polysubstituted by identical or different substituents from the group consisting of halogen, hydroxy, cyano, carboxy, C₁-C₆-alkyl-, C₁-C₆-alkoxy-, halo-C₁-C₆-alkyl-, halo-C₁-C₆-alkoxy-, C₃-C₁₀-cycloalkyl- and a monocyclic heterocyclyl radical having 3 to 8 ring atoms.

In the general formula I, preference is given to those compounds in which R² represents hydrogen or represents C₁-C₃-alkyl-.

In the general formula I, special preference is given to those compounds in which R² represents hydrogen or methyl.

In the general formula I, of special interest are compounds in which R³ represents hydrogen or represents C₁-C₆-alkyl-, C₁-C₆-alkylcarbonyl-, phenylsulphonyl- or C₁-C₆-alkylsulphonyl-, where the radicals mentioned may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, and in which X represents a carbon atom.

In the general formula I, of special interest are compounds in which R³ represents hydrogen or represents C₁-C₆-alkyl-, C₁-C₆-alkylcarbonyl-, phenylsulphonyl- or C₁-C₆-alkylsulphonyl-, where the radicals mentioned may optionally be mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, and in which X represents a nitrogen atom.

In the general formula I, preference is given to those compounds in which R³ represents hydrogen or represents C₁-C₃-alkyl- and in which X represents a carbon atom.

In the general formula I, preference is given to those compounds in which R³ represents hydrogen or represents C₁-C₃-alkyl- and in which X represents a nitrogen atom.

In the general formula I, special preference is given to those compounds in which R³ represents hydrogen or methyl and in which X represents a carbon atom.

In the general formula I, special preference is given to those compounds in which R³ represents hydrogen or methyl and in which X represents a nitrogen atom.

In the general formula I, of very particular interest are furthermore those compounds in which R² and

R³ together with the ring atoms N and X form a further heteroaromatic or heterocyclic ring having 5 to 7 ring atoms which may optionally be mono- or polysubstituted by identical or different substituents from the group consisting of halogen, amino, hydroxy, cyano, C₁-C₆-alkyl-, C₁-C₆-alkoxy-, halo-C₁-C₆-alkyl- and/or halo-C₁-C₆-alkoxy-.

In the general formula I, of particular interest are those compounds in which Y represents a spirocycloalkyl or heterospirocycloalkyl radical of 7 to 12 ring atoms, a bridged cycloalkyl radical or a bridged heterocycloalkyl radical of 7 to 12 ring atoms, where the radicals mentioned may optionally be mono- or polysubstituted by identical or different substituents from the group consisting of halogen, hydroxy, amino, oxo, cyano, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms.

In the general formula I, preference is given to those compounds in which Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned may optionally be mono- or polysubstituted by identical or different substituents from the group consisting of halogen, oxo, cyano, hydroxy, C₁-C₃-alkyl-, C₁-C₃-alkoxy-, halo-C₁-C₃-alkyl-, halo-C₁-C₃-alkoxy-, phenyl-, halophenyl-, phenyl-C₁-C₃-alkyl-, phenyl-C₁-C₃-alkoxy-, —C(═O)—NR⁶R⁷, —C(═O)—R⁸, —S(═O)₂—R⁹ and —NH—S(═O)₂—R⁹.

In the general formula I, even more preference is given to those compounds in which Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned may optionally be substituted by oxo, fluorine, chlorine, bromine, cyano, hydroxy, methyl, ethyl, methoxy, ethoxy, trifluoromethyl, benzyl, phenyl and/or —C(═O)—R⁸.

In the general formula I, particular preference is given to those compounds in which Y represents a spirocycloalkyl- or heterospirocycloalkyl radical of 7 to 11 ring atoms, a bridged cycloalkyl radical or a bridged heterocycloalkyl radical of 7 to 8 ring atoms, where the radicals mentioned may optionally be substituted by oxo, fluorine, chlorine, bromine, cyano, hydroxy, methyl, ethyl, methoxy, ethoxy, trifluoromethyl, benzyl, phenyl and/or —C(═O)—R⁸.

In the general formula I, special preference is given to those compounds in which Y represents a radical selected from

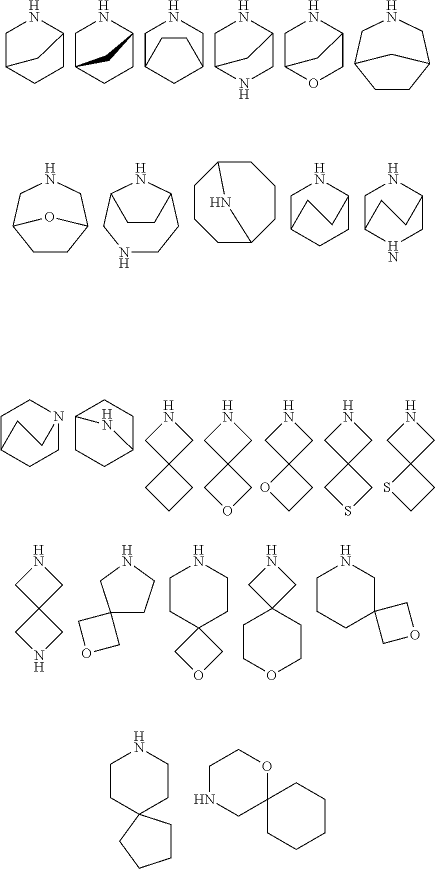

where the radicals mentioned above are optionally mono- or disubstituted independently of one another by identical or different substituents from the group consisting of oxo, fluorine, chlorine, bromine, cyano, hydroxy, methyl, ethyl, methoxy, ethoxy, benzyl, phenyl and —C(═O)—R⁸,

In the general formula I, very particular preference is given to those compounds in which Y represents a radical selected from

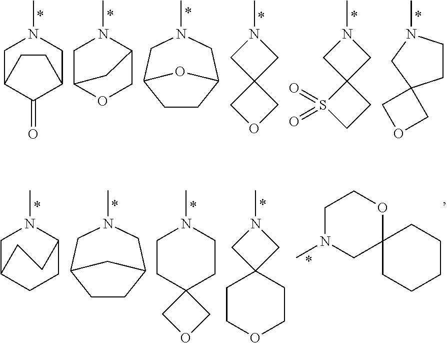

where “*” denotes the point of attachment to the remainder of the molecule.

In the general formula I, of particular interest are those compounds in which R⁶ and R⁷ independently of one another represent hydrogen, C₁-C₃-alkyl-, cyclopropyl- or di-C₁-C₃-alkylamino-C₁-C₃-alkyl-.

In the general formula I, preference is given to those compounds in which R⁶ and R⁷ independently of one another represent hydrogen or C₁-C₃-alkyl.

In the general formula I, of particular interest are those compounds in which R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl-having 5 or 6 ring atoms, where phenyl-, heteroaryl- and heterocyclyl- may optionally be mono- or disubstituted by halogen, C₁-C₃-alkoxy- or C₁-C₃-alkyl-.

In the general formula I, preference is given to those compounds in which R⁸ represents hydroxy, C₁-C₆-alkyl-, C₁-C₃-alkoxy-, halo-C₁-C₃-alkyl-, phenyl- or monocyclic heterocyclyl- having 3 to 8 ring atoms where phenyl- and heterocyclyl- may optionally be mono- or disubstituted by halogen, C₁-C₃-alkoxy- or C₁-C₃-alkyl-,

In the general formula I, particular preference is given to those compounds in which R⁸ represents hydroxy, C₁-C₆-alkyl- or halo-C₁-C₃-alkyl-.

In the general formula I, of particular interest are those compounds in which R⁹ represents C₁-C₆-alkyl-.

In the general formula I, preference is given to those compounds in which R⁹ represents C₁-C₃-alkyl-.

If X in the general formula I represents nitrogen, tautomeric forms of the compounds according to the invention may be possible. Here, the circle is supposed to represent both possible positions of the double bonds.

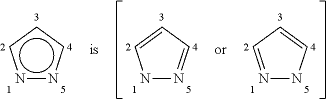

The invention is based on the following definitions:

### Alkyl

Alkyl represents a straight-chain or branched saturated monovalent hydrocarbon radical having generally 1 to 6 (C₁-C₆-alkyl), preferably 1 to 4 (C₁-C₄-alkyl) and particularly preferably 1 to 3 (C₁-C₃-alkyl) carbon atoms.

Examples which may be mentioned as being preferred are:

methyl-, ethyl-, propyl-, butyl-, pentyl-, hexyl-, isopropyl-, isobutyl-, sec-butyl-, tert-butyl-, isopentyl-, 2-methylbutyl-, 1-methylbutyl-, 1-ethylpropyl-, 1,2-dimethylpropyl-, neopentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, 1,2-dimethylbutyl-.

Particular preference is given to a methyl, ethyl, propyl, isopropyl or tert-butyl radical.

### Cycloalkyl

Cycloalkyl represents a monocyclic saturated monovalent hydrocarbon radical having generally 3 to 10 (C₃-C₁₀-cycloalkyl), preferably 3 to 8,

(C₃-C₈-cycloalkyl) and particularly preferably 3 to 7 (C₃-C₇-cycloalkyl) carbon atoms.

Examples of monocyclic cycloalkyl radicals which may be mentioned as being preferred are:

cyclopropyl, cyclobutyl, cyclopentyl, cyclohexyl and cycloheptyl.

Particular preference is given to a cyclopropyl, cyclopentyl or cyclohexyl radical.

### Alkoxy

Alkoxy represents a straight-chain or branched saturated alkylether radical of the formula —O-alkyl having generally 1 to 6 (C₁-C₆-alkoxy), preferably 1 to 4 (C₁-C₄-alkoxy) and particularly preferably 1 to 3 (C₁-C₃-alkoxy) carbon atoms.

Examples which may be mentioned as being preferred are:

methoxy, ethoxy, n-propoxy, isopropoxy, tert-butoxy, n-pentyloxy and n-hexyloxy.

### Alkoxyalkyl

Alkoxyalkyl represents an alkyl radical substituted by alkoxy, for example C₁-C₆-alkoxy-C₁-C₆-alkyl- or C₁-C₃-alkoxy-C₁-C₃-alkyl-.

Here, C₁-C₆-alkoxy-C₁-C₆-alkyl- means that the alkoxyalkyl group is attached via the alkyl moiety to the remainder of the molecule.

### Oxo

Oxo, an oxo group or an oxo substituent, is understood to mean a double-bonded oxygen atom ═O. Oxo may be attached to atoms of suitable valency, for example to a saturated carbon atom or to sulphur.

Preference is given to the bond to carbon with formation of a carbonyl group or to the bond to sulphur with formation of a sulphinyl or sulphonyl group.

### Alkylamino

Alkylamino represents an amino radical having one or two alkyl substituents (selected independently of one another) having generally 1 to 6 (C₁-C₆-alkylamino) and preferably 1 to 3 (C₁-C₃-alkylamino) carbon atoms.

(C₁-C₃)-Alkylamino represents, for example, a monoalkylamino radical having 1 to 3 carbon atoms or a dialkylamino radical having 1 to 3 carbon atoms each per alkyl substituent.

The following may be mentioned by way of example:

methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

### Alkylaminocarbonyl

Alkylaminocarbonyl represents the group alkylamino-C(═O)— having one or two alkyl substituents (selected independently of one another) having generally 1 to 6 (C₁-C₆-alkylaminocarbonyl) and preferably 1 to 3 (C₁-C₃-alkylaminocarbonyl) carbon atoms.

### Alkylcarbonyl

Alkylcarbonyl represents the group —C(═O)-alkyl having generally 1 to 6 (C₁-C₆-alkylcarbonyl), preferably 1 to 4 and particularly preferably 1 to 3 carbon atoms in the alkyl moiety.

Acetyl- and propanoyl may be mentioned by way of example.

### Alkylcarbonylamino

Alkylcarbonylamino represents the group alkyl-C(═O)—NH— having generally 1 to 6 (C₁-C₆-alkylcarbonylamino), preferably 1 to 4 and particularly preferably 1 to 3 carbon atoms in the alkyl moiety.

### Alkylsulphonyl

Alkylsulphonyl represents a straight-chain or branched saturated radical of the formula —S(═O)₂-alkyl having generally 1 to 6 (C₁-C₆-alkylsulphonyl), preferably 1 to 4 (C₁-C₄-alkylsulphonyl) and particularly preferably 1 to 3 (C₁-C₃-alkylsulphonyl) carbon atoms.

Examples which may be mentioned as being preferred are:

methylsulphonyl, ethylsulphonyl, propylsulphonyl.

Alkylaminosulphonyl

Alkylaminosulphonyl represents the group alkylamino-S(═O)₂— having one or two alkyl substituents (selected independently of one another) having generally 1 to 6 (C₁-C₆-alkylaminosulphonyl) and preferably 1 to 3 carbon atoms.

Examples which may be mentioned as being preferred are:

methylaminosulphonyl, ethylaminosulphonyl, dimethylaminosulphonyl.

### Phenylalkyl

Phenyl-C₁-C₆-alkyl- is understood to mean a group composed of an optionally substituted phenyl radical and a C₁-C₆-alkyl group, and bonded to the rest of the molecule via the C₁-C₆-alkyl group. Here, the alkyl radical has the meanings given above under alkyl.

Examples which may be mentioned include benzyl, phenethyl, phenylpropyl, phenylpentyl, with benzyl being preferred.

### Phenylalkoxy

Phenyl-C₁-C₆-alkoxy- is understood to mean a group composed of an optionally substituted phenyl radical and a C₁-C₆-alkoxy group, and bonded to the rest of the molecule via the C₁-C₆-alkoxy group. Here, the alkoxy radical has the meanings given above under alkoxy.

Examples which may be mentioned are benzoxy, phenethoxy, phenylpropyloxy, phenylpentyloxy, with benzoxy being preferred.

### Phenylsulphonyl

Phenylsulphonyl—is to be understood to mean a group composed of an optionally substituted phenyl radical and a —S(═O)₂ group.

Examples which may be mentioned are phenylsulphonyl, o- or p-toluylsulphonyl, m-chlorophenylsulphonyl.

### Heteroatoms

Heteroatoms are to be understood to mean oxygen, nitrogen or sulphur atoms.

### Heteroaryl

Heteroaryl means a monovalent aromatic ring system having 1, 2 or 3 heteroatoms. The heteroatoms may be nitrogen atoms, oxygen atoms and/or sulphur atoms. The binding valency can be at any aromatic carbon atom or at a nitrogen atom.

A monocyclic heteroaryl radical in accordance with the present invention has 5 or 6 ring atoms.

Heteroaryl radicals having 5 ring atoms include, for example, the rings:

thienyl, thiazolyl, furyl, pyrrolyl, oxazolyl, imidazolyl, pyrazolyl, isoxazolyl, isothiazolyl, oxadiazolyl, triazolyl, tetrazolyl and thiadiazolyl.

Heteroaryl radicals having 6 ring atoms include, for example, the rings:

pyridyl, pyridazinyl, pyrimidinyl, pyrazinyl and triazinyl.

### Monocyclic Heterocyclyl

Monocyclic heterocyclyl means a nonaromatic monocyclic ring system having 1, 2 or 3 heteroatoms. The heteroatoms may be nitrogen atoms, oxygen atoms and/or sulphur atoms.

A monocyclic heterocyclyl ring according to the present invention may have 3 to 8, preferably 4 to 7, particularly preferably 5 or 6 ring atoms.

By way of example and with preference, the following may be mentioned for monocyclic heterocyclyl radicals having 3 ring atoms:

aziridinyl.

By way of example and with preference, the following may be mentioned for monocyclic heterocyclyl radicals having 4 ring atoms:

azetidinyl, oxetanyl.

By way of example and with preference, the following may be mentioned for monocyclic heterocyclyl radicals having 5 ring atoms:

pyrrolidinyl, imidazolidinyl, pyrazolidinyl, pyrrolinyl, dioxolanyl and tetrahydrofuranyl.

By way of example and with preference, the following may be mentioned for monocyclic heterocyclyl radicals having 6 ring atoms:

piperidinyl, piperazinyl, morpholinyl, dioxanyl, tetrahydropyranyl and thiomorpholinyl.

By way of example and with preference, the following may be mentioned for monocyclic heterocyclyl radicals having 7 ring atoms:

azepanyl, oxepanyl, 1,3-diazepanyl, 1,4-diazepanyl.

By way of example and with preference, the following may be mentioned for monocyclic heterocyclyl radicals having 8 ring atoms:

oxocanyl, azocanyl.

From among the monocyclic heterocyclyl radicals, preference is given to 4- to 7-membered saturated heterocyclyl radicals having up to two heteroatoms from the group consisting of O, N and S. Particular preference is given to morpholinyl, piperidinyl and pyrrolidinyl.

### Spirocycloalkyl and Heterospirocycloalkyl

C₅-C₁₁-Spirocycloalkyl or C₅-C₁₁-heterospirocycloalkyl where 1-4 carbon atoms are replaced by heteroatoms as defined above in any combination is understood to mean a fusion of two saturated ring systems which share one common atom. Examples are spiro[2.2]pentyl, spiro[2.3]hexyl, azaspiro[2.3]hexyl, spiro[3.3]heptyl, azaspiro[3.3]heptyl, oxaazaspiro[3.3]heptyl, thiaazaspiro[3.3]heptyl, oxaspiro[3.3]heptyl, oxazaspiro[5.3]nonyl, oxazaspiro[4.3]octyl, oxazaspiro[5.5]undecyl, diazaspiro[3.3]heptyl, thiazaspiro[3.3]heptyl, thiazaspiro[4.3]octyl, azaspiro[5.5]decyl, and the further homologous spiro[3.4], spiro[4.4], spiro[5.5], spiro[6.6], spiro[2.4], spiro[2.5], spiro[2.6], spiro[3.5], spiro[3.6], spiro[4.5], spiro[4.6] and spiro[5.6] systems including the variants modified by heteroatoms according to the definition. Preference is given to C₆-C₈-heterospirocycloalkyl-.

### Bridged Cycloalkyl and Bridged Heterocycloalkyl

A bridged C₆-C₁₂ ring system such as bridged C₆-C₁₂-cycloalkyl- or bridged C₆-C₁₂-heterocycloalkyl- is understood to mean a fusion of at least two saturated rings which share two atoms that are not directly adjacent to one another. This may give rise either to a bridged carbocycle (bridged cycloalkyl) or to a bridged heterocycle (bridged heterocycloalkyl) where 1-4 carbon atoms are replaced by heteroatoms as defined above in any combination. Examples are bicyclo[2.2.1]heptyl-, azabicyclo[2.2.1]heptyl-, oxazabicyclo[2.2.1]heptyl-, thiazabicyclo[2.2.1]heptyl-, diazabicyclo[2.2.1]heptyl-, bicyclo[2.2.2]octyl-, azabicyclo[2.2.2]octyl-, diazabicyclo[2.2.2]octyl-, oxazabicyclo[2.2.2]octyl-, thiazabicyclo[2.2.2]octyl-, bicyclo[3.2.1]octyl-, azabicyclo[3.2.1]octyl-, diazabicyclo[3.2.1]octyl-, oxazabicyclo[3.2.1]octyl-, thiazabicyclo[3.2.1]octyl-, bicyclo[3.3.1]nonyl-, azabicyclo[3.3.1]nonyl, diazabicyclo[3.3.1]nonyl- oxazabicyclo[3.3.1]nonyl-, thiazabicyclo[3.3.1]nonyl-, bicyclo[4.2.1]nonyl-, azabicyclo[4.2.1]nonyl-, diazabicyclo[4.2.1]nonyl-, oxazabicyclo[4.2.1]nonyl-, thiazabicyclo[4.2.1]nonyl-, bicyclo[3.3.2]decyl-, azabicyclo[3.3.2]decyl-, diazabicyclo[3.3.2]decyl-, oxazabicyclo[3.3.2]decyl-, thiazabicyclo[3.3.2]decyl- or azabicyclo[4.2.2]decyl- and the further possible combinations according to the definition. Preference is given to bridged C₆-C₁₀-heterocycloalkyl-.

### Halogen

The term “halogen” includes fluorine, chlorine, bromine and iodine.

Preference is given to fluorine, chlorine and bromine, in particular fluorine or chlorine.

### Haloalkyl:

Haloalkyl represents an alkyl radical having at least one halogen substituent.

A halo-C₁-C₆-alkyl radical is an alkyl radical having 1-6 carbon atoms and at least one halogen substituent. If a plurality of halogen substituents is present, these may also be different from one another.

Examples which may be mentioned as being preferred are:

the trifluoromethyl, 2,2,2-trifluoroethyl, pentafluoroethyl, 4,4,5,5,5-pentafluoropentyl or 3,3,4,4,5,5,5-heptafluoropentyl group.

Preference is given to perfluorinated alkyl radicals such as trifluoromethyl or pentafluoroethyl.

### Haloalkoxy

Haloalkoxy represents an alkoxy radical having at least one halogen substituent.

A halo-C₁-C₆-alkoxy radical is an alkoxy radical having 1-6 carbon atoms and at least one halogen substituent. If a plurality of halogen substituents is present, these may also be different from one another. Preference is given to fluoroalkoxy radicals.

Examples which may be mentioned as being preferred are:

the trifluoromethoxy or 2,2,2-trifluoroethoxy radical.

### Hydroxyalkyl

Haloalkyl represents an alkyl radical having at least one hydroxy substituent.

A hydroxy-C₁-C₆-alkyl radical is an alkyl radical having 1-6 carbon atoms and at least one hydroxy substituent.

### Aminoalkyl

Aminoalkyl represents an alkyl radical having at least one amino substituent.

An amino-C₁-C₆-alkyl radical is an alkyl radical consisting of 1-6 carbon atoms and at least one amino substituent.

### Alkylaminoalkyl

Alkylaminoalkyl—represents an alkyl radical substituted by alkylamino as defined above, for example C₁-C₆-alkylamino-C₁-C₆-alkyl- or C₁-C₃-alkylamino-C₁-C₃-alkyl-.

Here, C₁-C₆-alkylamino-C₁-C₆-alkyl- means that the alkylaminoalkyl group is attached via the alkyl moiety to the remainder of the molecule.

Dialkylaminoalkyl-, for example di-C₁-C₃-alkylamino-C₁-C₃-alkyl-, means, that the alkylamino moiety mentioned above obligatorily contains two alkyl groups which may be identical or different.

Examples of alkylaminoalkyl are N,N-dimethylaminoethyl-, N,N-dimethylaminomethyl-, N,N-diethylaminoethyl-, N,N-dimethylaminopropyl-, N-methylaminoethyl-, N-methylaminomethyl-.

The specific radical definitions given in the particular combinations or preferred combinations of radicals are, irrespective of the particular combinations of radicals specified, also replaced as desired by radical definitions of other combination.

Very particular preference is given to combinations of two or more of the abovementioned preferred ranges.

Compounds according to the invention are the compounds of the formula (I) and their salts, solvates and solvates of the salts, the compounds encompassed by formula (I) of the formulae mentioned below and their salts, solvates and solvates of the salts and the compounds encompassed by formula (I) and mentioned below as working examples, and their salts, solvates and solvates of the salts, if the compounds encompassed by formula (I) and mentioned below are not already salts, solvates and solvates of the salts.

The present invention is likewise considered to encompass the use of the salts of the compounds according to the invention.

Preferred salts in the context of the present invention are physiologically acceptable salts of the compounds according to the invention. The invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention furthermore include base addition salts, for example of alkali metals such as sodium and potassium, of alkaline earth metals such as calcium and magnesium, or of ammonium salts derived from ammonia or organic amines having 1 to 16 carbon atoms, for example methylamine, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base and/or 1-amino-2,3,4-butanetriol. Furthermore, the compounds according to the invention may form base addition salts with quarterary ammonium ions which can be obtained, for example, by quarternization of corresponding amines with agents such as lower alkyl halides, for example methyl-, ethyl-, propyl- and butyl chlorides, bromides and iodides, dialkyl sulphates such as dimethyl, diethyl, dibutyl and diamyl sulphate, long-chain halides such as decyl, lauryl, myristyl and stearyl chlorides, bromides and iodides, or arylalkyl halides such as benzyl bromide or phenethyl bromide. Examples of such quarternary ammonium ions are tetramethylammonium, tetraethylammonium, tetra(n-propyl)ammonium, tetra(n-butyl)ammonium and also benzyltrimethylammonium.

The present invention further provides all the possible crystalline and polymorphous forms of the compounds according to the invention, where the polymorphs may be present either as single polymorphs or as a mixture of a plurality of polymorphs in all concentration ranges.

The present invention furthermore provides medicaments comprising the compounds according to the invention and at least one or more further active compounds, in particular for the prophylaxis and/or therapy of neoplastic disorders.

Solvates in the context of the invention are described as those forms of the compounds according to the invention which form a complex in the solid or liquid state by coordination with solvent molecules. Hydrates are a specific form of the solvates in which the coordination is with water. Solvates preferred in the context of the present invention are hydrates.

The compounds according to the invention may, depending on their structure, exist in different stereoisomeric forms, i.e. in the form of configurational isomers or else optionally as conformational isomers. The compounds according to the invention have, at the

carbon atom (C-4) of the diazepine skeleton attached to Y via —(CH₂)—C(═O)—, a centre of asymmetry. They may therefore take the form of pure enantiomers, racemates, or else of diastereomers or mixtures thereof when one or more of the substituents described in the formula (I) contains a further element of asymmetry, for example a chiral carbon atom. The present invention therefore also encompasses the enantiomers and diastereomers, and the respective mixtures thereof. The pure enantiomers and diastereomers can be isolated from such mixtures in a known manner; chromatography processes are preferably used for this, in particular HPLC chromatography on a chiral or achiral phase.

In general, the enantiomers according to the invention inhibit the target to different degrees and have different activity in the cancer cell lines studied. The more active enantiomer, which frequently is the 4S enantiomer, is preferred.

Where the compounds according to the invention can occur in tautomeric forms, the present invention encompasses all the tautomeric forms.

The present invention also encompasses all suitable isotopic variants of the compounds according to the invention. An isotopic variant of an inventive compound is understood here to mean a compound in which at least one atom within the inventive compound has been exchanged for another atom of the same atomic number but with a different atomic mass from the atomic mass which usually or predominantly occurs in nature. Examples of isotopes which can be incorporated into a compound according to the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁵S, ³⁶S, ¹⁸F, ³⁶Cl, ⁸²BR, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active compound distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the compounds according to the invention may therefore in some cases also constitute a preferred embodiment of the present invention. Isotopic variants of the compounds according to the invention can be prepared by the processes known to those skilled in the art, for example by the methods described below and the procedures described in the working examples, by using corresponding isotopic modifications of the respective reagents and/or starting compounds.

In addition, the present invention also encompasses prodrugs of the compounds according to the invention. The term “prodrugs” includes compounds which may themselves be biologically active or inactive but are converted to compounds according to the invention while resident in the body (for example metabolically or hydrolytically).

The compounds according to the invention can act systemically and/or locally. For this purpose, they can be administered in a suitable manner, for example by the oral, parenteral, pulmonary, nasal, sublingual, lingual, buccal, rectal, dermal, transdermal, conjunctival or otic route, or as implant or stent.

The compounds according to the invention can be administered in suitable administration forms for these administration routes.

Suitable administration forms for oral administration are those which function according to the prior art and deliver the compounds according to the invention rapidly and/or in modified fashion, and which contain the compounds according to the invention in crystalline and/or amorphized and/or dissolved form, for example tablets (uncoated or coated tablets, for example having enteric coatings or coatings which are insoluble or dissolve with a delay and control the release of the compound according to the invention), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds according to the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising the compounds according to the invention, typically together with one or more inert, nontoxic, pharmaceutically suitable excipients, and for the use thereof for the aforementioned purposes.

The compounds according to the invention are formulated to give pharmaceutical preparations in a manner known per se, by converting the active compound(s) to the desired administration form with the excipients customary in pharmaceutical formulation.

The excipients used may, for example, be carrier substances, fillers, disintegrants, binders, humectants, glidants, absorbents and adsorbents, diluents, solvents, cosolvents, emulsifiers, solubilizers, taste correctors, colourants, preservatives, stabilizers, wetting agents, salts for modifying the osmotic pressure or buffers. Reference should be made to Remington's Pharmaceutical Science, 15th ed. Mack Publishing Company, East Pennsylvania (1980).

The pharmaceutical formulations can be present

in solid form, for example as tablets, sugar-coated tablets, pills, suppositories, capsules, transdermal systems or
in semisolid form, for example as ointments, creams, gels, suppositories, emulsions or
in liquid form, for example as solutions, tinctures, suspensions or emulsions.

Excipients in the context of the invention may, for example, be salts, saccharides (mono-, di-, tri-, oligo- and/or polysaccharides), proteins, amino acids, peptides, fats, waxes, oils, hydrocarbons and derivatives thereof, and the excipients may be of natural origin or be obtained by synthetic or partially synthetic means.

Useful forms for oral or peroral administration are especially tablets, coated tablets, capsules, pills, powders, granules, pastilles, suspensions, emulsions or solutions.

Useful forms for parenteral administration are especially suspensions, emulsions, and particularly solutions.

The present invention relates to the compounds according to the invention.

They can be used for the prophylaxis and therapy of human disorders, in particular neoplastic disorders.

The compounds according to the invention can be used in particular for inhibiting or reducing cell proliferation and/or cell division and/or to induce apoptosis.

The compounds according to the invention are suitable in particular for the prophylaxis and/or therapy of hyper-proliferative disorders such as, for example,
psoriasis,
keloids and other skin hyperplasias,
benign prostate hyperplasias (BPH),
solid tumours and
haematological tumours.

Solid tumours that can be treated in accordance with the invention are, for example, tumours of the breast, the respiratory tract, the brain, the reproductive organs, the gastrointestinal tract, the urogenital tract, the eye, the liver, the skin, the head and the neck, the thyroid gland, the parathyroid gland, the bones, and the connective tissue and metastases of these tumours.

Haematological tumours which can be treated are, for example,
multiple myelomas
lymphomas or
leukaemias

Breast tumours which can be treated are, for example:
breast carcinomas with positive hormone receptor status
breast carcinomas with negative hormone receptor status
Her-2 positive breast carcinomas
hormone receptor and Her-2 negative breast carcinomas
BRCA-associated breast carcinomas
inflammatory breast carcinomas.

Tumours of the respiratory tract which can be treated are, for example,
non-small-cell bronchial carcinomas such as, for example, squamous cell carcinoma, adenocarcinoma, large-cell carcinoma and
small-cell bronchial carcinomas.

Tumours of the brain which can be treated are, for example,
gliomas,
glioblastomas,
astrocytomas,
meningiomas and
medulloblastomas.

Tumours of the male reproductive organs which can be treated are, for example:
prostate carcinomas,
malignant epididymal tumours
malignant testicular tumours and
penis carcinomas.

Tumours of the female reproductive organs which can be treated are, for example:
endometrial carcinomas
cervix carcinomas
ovarian carcinomas
vaginal carcinomas
vulvar carcinomas

Tumours of the gastrointestinal tract which can be treated are, for example:
colorectal carcinomas
anal carcinomas
stomach carcinomas
pancreas carcinomas
oesophagus carcinomas
gall bladder carcinomas
carcinomas of the small intestine
salivary gland carcinomas
neuroendocrine tumours
gastrointestinal stroma tumours

Tumours of the uorgenital tract which can be treated are, for example:
urinary bladder carcinomas
kidney cell carcinomas
carcinomas of the renal pelvis and lower urinary tract

Tumours of the eye which can be treated are, for example:
retinoblastomas
intraocular melanomas

Tumours of the liver which can be treated are, for example:
hepatocellular carcinomas
cholangiocellular carcinomas

Tumours of the skin which can be treated are, for example:
malignant melanomas
basaliomas
spinaliomas
Kaposi sarcomas
Merkel cell carcinomas

Tumours of the head and neck which can be treated are, for example:
larynx carcinomas
carcinomas of the pharynx and the oral cavity
carcinomas of the middle line structures (e.g. NMC, C. A. French, Annu. Rev. Pathol. 2012, 7:247-265)

Sarcomas which can be treated are, for example:
soft tissue sarcomas
osteosarcomas

Lymphomas which can be treated are, for example:
non-Hodgkin lymphomas
Hodgkin lymphomas
cutaneous lymphomas
lymphomas of the central nervous system
AIDS-associated lymphomas

Leukaemias which can be treated are, for example:
acute myeloid leukaemias
chronic myeloid leukaemias
acute lymphatic leukaemias
chronic lymphatic leukaemias
hairy cell leukaemias

Advantageously, the compounds according to the invention can be used for prophylaxis and/or therapy of leukaemias, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

Particularly advantageously, the compounds according to the invention can be used for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The compounds according to the invention are also suitable for prophylaxis and/or therapy of benign hyperproliferative diseases, for example endometriosis, leiomyoma and benign prostate hyperplasia.

The compounds according to the invention are also suitable for male fertility control.

The compounds according to the invention are also suitable for prophylaxis and/or therapy of systemic inflammatory diseases, especially LPS-induced endotoxic shock and/or bacteria-induced sepsis.

The compounds according to the invention are also suitable for prophylaxis and/or therapy of inflammatory or autoimmune disorders, for example:
pulmonary disorders associated with inflammatory, allergic and/or proliferative processes: chronic obstructive pulmonary disorders of any origin, particularly bronchial asthma; bronchitis of different origin; all forms of restrictive pulmonary disorders, particularly allergic alveolitis; all forms of pulmonary oedema, particularly toxic pulmonary oedema; sarcoidoses and granulomatoses, particularly Boeck's disease,
rheumatic disorders/autoimmune disorders/joint disorders associated with inflammatory, allergic and/or proliferative processes: all forms of rheumatic disorders, especially rheumatoid arthritis, acute rheumatic fever, polymyalgia rheumatica; reactive arthritis; inflammatory soft-tissue disorders of other origin; arthritic symptoms in the case of degenerative joint disorders (arthroses); traumatic arthritides; collagenoses of any origin, e.g. systemic lupus erythematosus, scleroderma, polymyositis, dermatomyositis, Sjögren's syndrome, Still's syndrome, Felty's syndrome
allergies associated with inflammatory and/or proliferative processes: all forms of allergic reactions, e.g. angiooedema, hay fever, insect bites, allergic reactions to medicaments, blood derivatives, contrast agents, etc., anaphylactic shock, urticaria, contact dermatitis
vascular inflammation (vasculitis): panarteritis nodosa, temporal arteritis, erythema nodosum
dermatological disorders associated with inflammatory, allergic and/or proliferative processes: atopic dermatitis; psoriasis; pityriasis rubra pilaris; erythematous disorders triggered by different noxae, for example radiation, chemicals, burns, etc.; bullous dermatoses; lichenoid disorders; pruritus; seborrhoeic eczema; rosacea; pemphigus vulgaris; erythema exsudativum multiforme; balanitis; vulvitis; hair loss, such as alopecia areata; cutaneous T-cell lymphoma
renal disorders associated with inflammatory, allergic and/or proliferative processes: nephrotic syndrome; all nephritides
hepatic disorders associated with inflammatory, allergic and/or proliferative processes: acute hepatic disintegration; acute hepatitis of different origin, for example viral, toxic, medicament-induced; chronic aggressive and/or chronic intermittent hepatitis
gastrointestinal disorders associated with inflammatory, allergic and/or proliferative processes: regional enteritis (Crohn's disease); ulcerative colitis; gastritis; reflux oesophagitis; gastroenteritides of other origin, e.g. indigenous sprue
proctological disorders associated with inflammatory, allergic and/or proliferative processes: anal eczema; fissures; haemorrhoids; idiopathic proctitis,
ocular disorders associated with inflammatory, allergic and/or proliferative processes: allergic keratitis, uveitis, iritis; conjunctivitis; blepharitis; optic neuritis; chlorioditis; sympathetic ophthalmia
disorders of the ear-nose-throat region associated with inflammatory, allergic and/or proliferative processes: allergic rhinitis, hay fever; otitis externa, for example caused by contact eczema, infection, etc.; otitis media
neurological disorders associated with inflammatory, allergic and/or proliferative processes: cerebral oedema, particularly tumour-related cerebral oedema; multiple sclerosis; acute encephalomyelitis; meningitis; various forms of seizure, for example West's syndrome
haematological disorders associated with inflammatory, allergic and/or proliferative processes: congenital haemolytic anaemia; idiopathic thrombocytopenia
neoplastic disorders associated with inflammatory, allergic and/or proliferative processes: acute lymphatic leukaemia; malignant lymphoma; lymphogranulomatoses; lymphosarcoma; extensive metastases, particularly in the case of mammary, bronchial and prostate carcinoma
endocrine disorders associated with inflammatory, allergic and/or proliferative processes: endocrine orbitopathy; thyrotoxic crisis; de Quervain's thyroiditis; Hashimoto's thyroiditis; Basedow's disease
organ and tissue transplants, graft-versus-host disease
severe states of shock, for example anaphylactic shock, systemic inflammatory response syndrome (SIRS)
substitution therapy in the case of: congenital primary adrenal insufficiency, for example congenital adrenogenital syndrome; acquired primary adrenal insufficiency, for example Addison's disease, autoimmune adrenalitis, postinfectious, tumours, metastases, etc; congenital secondary adrenal insufficiency, for example congenital hypopituitarism; acquired secondary adrenal insufficiency, for example postinfectious, tumours, etc.
emesis associated with inflammatory, allergic and/or proliferative processes, for example in combination with a 5-HT3 antagonist in the case of cytostatic-induced vomiting
pain of inflammatory origin, for example lumbago

The compounds according to the invention are also suitable for the treatment of viral disorders, for example infections caused by papilloma viruses, herpes viruses, Epstein-Barr viruses, hepatitis B or C viruses, and human immunodeficiency viruses.

The compounds according to the invention are also suitable for the treatment of atherosclerosis, dyslipidaemia, hypercholesterolaemia, hypertriglyceridaemia, peripheral vascular disorders, cardiovascular disorders, angina pectoris, ischaemia, stroke, myocardial infarction, angioplastic restenosis, hypertension, thrombosis, obesity, endotoxaemia.

The compounds according to the invention are also suitable for the treatment of neurodegenerative diseases, for example multiple sclerosis, Alzheimer's disease and Parkinson's disease.

These disorders are well characterized in man, but also exist in other mammals.

The present application further provides the compounds according to the invention for use as medicaments, especially for prophylaxis and/or therapy of neoplastic disorders.

The present application further provides the inventive compounds for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the compounds according to the invention for production of a medicament.

The present application further provides for the use of the compounds according to the invention for production of a medicament for prophylaxis and/or therapy of neoplastic disorders.

The present application further provides for the use of the compounds according to the invention for production of a medicament for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the compounds according to the invention for production of a medicament for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides for the use of the compounds according to the invention for prophylaxis and/or therapy of neoplastic disorders.

The present application further provides for the use of the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides for the use of the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The present application further provides pharmaceutical formulations in the form of tablets comprising one of the compounds according to the invention for prophylaxis and/or therapy of leukaemia, especially acute myeloid leukaemia, prostate carcinoma, especially androgen receptor-positive prostate carcinoma, cervical carcinoma, mammary carcinoma, especially hormone receptor-negative, hormone receptor-positive or BRCA-associated mammary carcinoma, pancreatic carcinoma, renal cell carcinoma, hepatocellular carcinoma, melanoma and other skin tumours, non-small-cell bronchial carcinoma, endometrial carcinoma and colorectal carcinoma.

The present application further provides pharmaceutical formulations in the form of tablets comprising one of the compounds according to the invention for prophylaxis and/or therapy of leukaemia, especially acute myeloid leukaemia, prostate carcinoma, especially androgen receptor-positive prostate carcinoma, mammary carcinoma, especially oestrogen receptor alpha-negative mammary carcinoma, melanoma or multiple myeloma.

The invention further provides for the use of the compounds according to the invention for treatment of disorders associated with proliferative processes.

The invention further provides for the use of the compounds according to the invention for treatment of benign hyperplasias, inflammation disorders, autoimmune disorders, sepsis, viral infections, vascular disorders and neurodegenerative disorders.

The compounds according to the invention can be used alone or, if required, in combination with one or more other pharmacologically active substances, provided that this combination does not lead to undesirable and unacceptable side effects. Accordingly, the present invention further provides medicaments comprising a compound according to the invention and one or more further active compounds, in particular for the prophylaxis and/or therapy of the disorders mentioned above.

For example, the compounds according to the invention can be combined with known antihyperproliferative, cytostatic or cytotoxic substances for treatment of cancer. The combination of the compounds according to the invention with other substances commonly used for cancer treatment, or else with radiotherapy, is particularly appropriate.

An illustrative but nonexhaustive list of suitable combination active compounds is as follows: abiraterone acetate, abraxane, acolbifene, Actimmune, actinomycin D (dactinomycin), afatinib, affinitak, Afinitor, aldesleukin, alendronic acid, alfaferone, alitretinoin, allopurinol, Aloprim, Aloxi, alpharadin, altretamine, aminoglutethimide, aminopterin, amifostine, amrubicin, amsacrine, anastrozole, anzmet, apatinib, Aranesp, arglabin, arsenic trioxide, Aromasin, arzoxifen, asoprisnil, L-asparaginase, atamestane, atrasentane, avastin, axitinib, 5-azacytidine, azathioprine, BCG or Tice BCG, bendamustine, bestatin, beta-methasone acetate, betamethasone sodium phosphate, bexarotene, bicalutamide, bleomycin sulphate, broxuridine, bortezomib, bosutinib, busulfan, cabazitaxel, calcitonin, campath, camptothecin, capecitabine, carboplatin, carfilzomib, carmustine, casodex, CCI-779, CDC-501, cediranib, cefesone, celebrex, celmoleukin, cerubidine, cediranib, chlorambucil, cisplatin, cladribine, clodronic acid, clofarabine, colaspase, copanlisib, corixa, crisnatol, crizotinib, cyclophosphamide, cyproterone acetate, cytarabine, dacarbazine, dactinomycin, dasatinib, daunorubicin, DaunoXome, Decadron, Decadron Phosphate, decitabine, degarelix, delestrogen, denileukin diftitox, depomedrol, deslorelin, dexrazoxane, diethylstilbestrol, diflucan, 2′,2′-difluorodeoxycytidine, DN-101, docetaxel, doxifluridine, doxorubicin (Adriamycin), dronabinol, dSLIM, dutasteride, DW-166HC, edotecarin, eflornithine, Eligard, Elitek, Ellence, Emend, enzalutamide, epirubicin, epoetin-alfa, Epogen, epothilone and derivatives thereof, eptaplatin, ergamisol, erlotinib, erythro-hydroxynonyladenine, estrace, oestradiol, oestramustine sodium phosphate, ethinyloestradiol, Ethyol, etidronic acid, etopophos, etoposide, everolimus, exatecan, exemestane, fadrozole, farston, fenretinide, filgrastim, finasteride, fligrastim, floxuridine, fluconazole, fludarabine, 5-fluorodeoxyuridine monophosphate, 5-fluorouracil (5-FU), fluoxymesterone, flutamide, folotin, formestane, fosteabine, fotemustine, fulvestrant, Gammagard, gefitinib, gemcitabine, gemtuzumab, Gleevec, Gliadel, goserelin, gossypol, granisetrone hydrochloride, hexamethylmelamine, histamine dihydrochloride, histrelin, holmium-166-DOTPM, hycamtin, hydrocortone, erythro-hydroxynonyladenine, hydroxyurea, hydroxyprogesterone caproate, ibandronic acid, ibritumomab tiuxetan, idarubicin, ifosfamide, imatinib, iniparib, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, ixabepilone, keyhole limpet haemocyanin, kytril, lanreotide, lapatinib, lasofoxifene, lentinan sulphate, lestaurtinib, letrozole, leucovorin, leuprolide, leuprolide acetate, levamisole, levofolic acid calcium salt, levothroid, levoxyl, Libra, liposomal MTP-PE, lomustine, lonafarnib, lonidamine, marinol, mechlorethamine, mecobalamine, medroxyprogesterone acetate, megestrol acetate, melphalan, Menest, 6-mercaptopurine, mesna, methotrexate, metvix, miltefosine, minocycline, minodronate, miproxifen, mitomycin C, mitotan, mitoxantrone, modrenal, MS-209, MX-6, myocet, nafarelin, nedaplatin, nelarabine, nemorubicin, neovastat, neratinib, neulasta, neumega, neupogen, nilotimib, nilutamide, nimustine, nolatrexed, nolvadex, NSC-631570, obatoclax, oblimersen, OCT-43, octreotide, olaparib, ondansetron hydrochloride, Onco-TCS, Orapred, Osidem, oxaliplatin, paclitaxel, pamidronate disodium, pazopanib, pediapred, pegaspargase, pegasys, pemetrexed, pentostatin, N-phosphonoacetyl-L-aspartate, picibanil, pilocarpine hydrochloride, pirarubicin, plerixafor, plicamycin, PN-401, porfimer sodium, prednimustine, prednisolone, prednisone, Premarin, procarbazine, Procrit, QS-21, quazepam, R-1589, raloxifene, raltitrexed, ranpirnas, RDEA119, Rebif, regorafenib, 13-cis-retinoic acid, rhenium-186 etidronate, rituximab, roferon-A, romidepsin, romurtide, ruxolitinib, salagen, salinomycin, sandostatin, sargramostim, satraplatin, semaxatinib, semustine, seocalcitol, sipuleucel-T, sizofiran, sobuzoxan, Solu-Medrol, sorafenib, streptozocin, strontium-89 chloride, sunitinib, Synthroid, T-138067, tamoxifen, tamsulosin, Tarceva, tasonermin, tastolactone, Taxoprexin, Taxoter, teceleukin, temozolomide, temsirolimus, teniposide, testosterone propionate, Testred, thalidomide, thymosin alpha-1, thioguanine, thiotepa, thyrotropin, tiazorufin, tiludronic acid, tipifarnib, tirapazamine, TLK-286, toceranib, topotecan, toremifen, tositumomab, tastuzumab, teosulfan, transMID-107R, tretinoin, Trexall, trimethylmelamine, trimetrexate, triptorelin acetate, triptorelin pamoate, trofosfamide, UFT, uridine, valrubicin, valspodar, vandetanib, vapreotide, vatalanib, vemurafinib, verte-porfin, vesnarinone, vinblastine, vincristine, vindesine, vinflumine, vinorelbine, virulizin, vismodegib, Xeloda, Z-100, Zinecard, zinostatin stimalamer, zofran, zoledronic acid.

The combination of the compound according to the invention with a P-TEFb or CDK9 inhibitor is likewise particularly preferred.

In a promising manner, the compounds according to the invention can also be combined with biologics such as antibodies (for example aflibercept, alemtuzumab, bevacizumab, brentuximumab, catumaxomab, cetuximab, denosumab, edrecolomab, gemtuzumab, ibritumomab, ipilimumab, ofatumumab, panitumumab, pertuzumab, rituximab, tositumumab, trastuzumab) and recombinant proteins.

The compounds according to the invention can also achieve positive effects in combination with other therapies directed against angiogenesis, for example with bevacizumab, axitinib, regorafenib, cediranib, sorafenib, sunitinib or thalidomide. Combinations with antihormones and steroidal metabolic enzyme inhibitors are particularly suitable because of their favourable profile of side effects.

Generally, the following aims can be pursued with the combination of the compounds according to the invention with other cytostatically or cytotoxically active agents:
improved efficacy in slowing the growth of a tumour, in reducing its size or even in the complete elimination thereof, compared with treatment with an individual active compound;
the possibility of using the chemotherapeutics used in a lower dosage than in the case of monotherapy;
the possibility of a more tolerable therapy with fewer side effects compared with individual administration;
the possibility of treatment of a broader spectrum of tumours;
the achievement of a higher rate of response to the therapy;
a longer survival time of the patient compared with present-day standard therapy.

In addition, the compounds according to the invention can also be used in conjunction with radiotherapy and/or surgical intervention.

### Preparation of the Compounds According to the Invention

The preparation of the compounds of the general formula (III) is described in an exemplary manner by the schemes below:

4-Aminopyrrolobenzophenones can be prepared by a reaction sequence shown in Scheme 1.

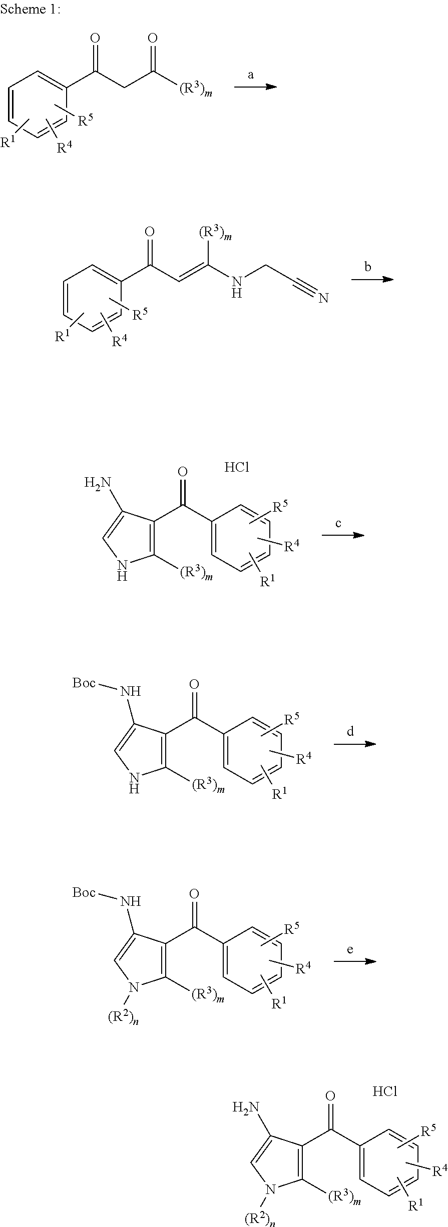

Here, R¹, R², R³, R⁴ and R⁵ as well as n and m have the meanings given under the General Formula I.

a) 2-aminoacetonitrile, base, solvent, reflux, removal of water; b) EtOH, base, then HCl*dioxane; c) Boc₂O, base; d) R² LG, base, opt. catalyst; e) z.B. HCl*dioxane

The reaction sequence a) and b) for cyclizing the pyrrole is a sequence known to the person skilled in the art (Il Farmaco, Edizione Scientifica (1984), 39, p. 538ff, Tarzia et al.). By reaction with corresponding alkyl halides or alkyl sulphates in Step d), it is possible to introduce alkyl substituents R² in accordance with the general formula (I) using methods known to the person skilled in the art. By reaction with acyl halides or acyl anhydrides or aryl- and alkylsulphonyl chlorides, it is possible to introduce acyl or aryl-oder alkylsulphonyl substituents as R² according to the general formula (I) using methods known to the person skilled in the art. Aryl- and heteroaryl radicals as R² can be introduced by reaction with the corresponding aryl- or heteroaryl halides and a palladium or copper transition metal catalyst (J. Am. Chem. Soc. (1998), 120, S. 827-8, Hartwig et al.; Bioorg. Med Chem. Lett. (2011), 21, p. 4306ff, Xie et al.). Here, LG is to be understood as a leaving group which, as described herein, may, for example, be a halogen or a boronic acid.

4-Aminopyrazolobenzophenones can be prepared by a reaction sequence shown in Scheme 2.

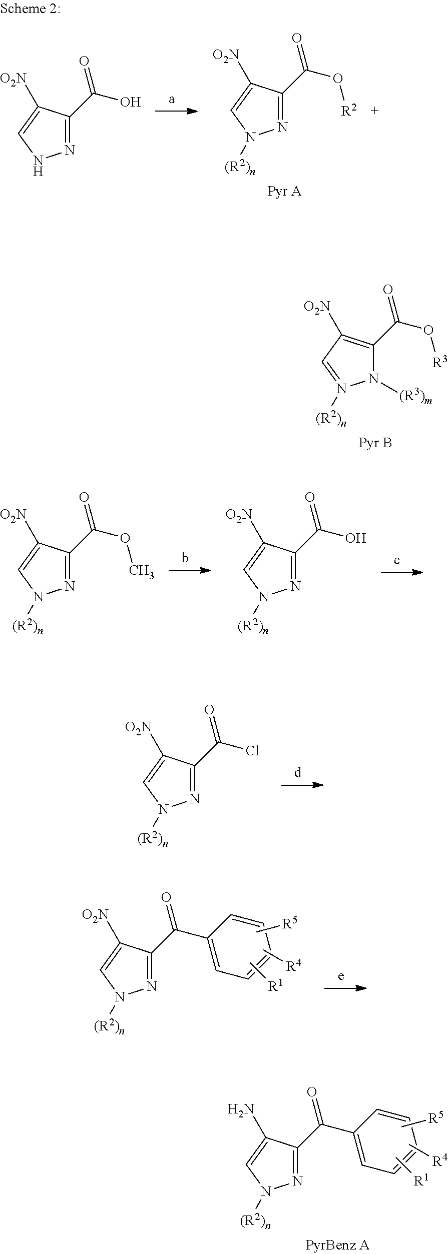

Here, R¹, R², R³, R⁴ and R⁵ as well as n and m have the meanings given under the General Formula I.

a) R² halogen, K₂CO₃, DMF; b) NaOH, MeOH, water; c) oxalyl chloride, POCl₃ or PCl₃; d) ArR¹R⁴R⁵, AlCl₃; e) Fe, NH₄Cl, water, EtOH

The reaction sequence a) to e) in Scheme 2 has been described (J Med. Chem. (1973), 16, p. 1346ff, DeWald et al.) and can be carried out analogously. By reaction with corresponding alkyl halides or alkyl sulphates in Step a), it is possible to introduce alkyl substituents R² in accordance with the general formula (I) using methods known to the person skilled in the art. By reaction with acyl halides or acyl anhydrides or aryl- and alkylsulphonyl chlorides, it is possible to introduce acyl or aryl-oder alkylsulphonyl substituents as R² according to the general formula (I) using methods known to the person skilled in the art. Aryl- and heteroaryl radicals as R² can be introduced by reaction with the corresponding aryl- or heteroaryl halides and a palladium or copper transition metal catalyst (J. Am. Chem. Soc. (1998), 120, p. 827-8, Hartwig et al.; Bioorg. Med Chem. Lett. (2011), 21, p. 4306ff, Xie et al.). Here, LG is to be understood as a leaving group which, as described herein, may, for example, be a halogen or a boronic acid.

The pyrazoles Pyr A and Pyr B generated in Scheme 2, Step a) are, separately, converted into PyrBenz A and PyrBenz B by the reaction sequence according to Scheme 2.

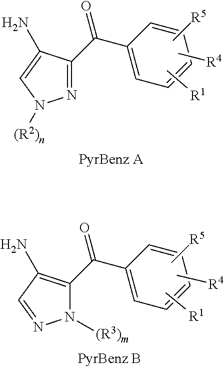

Here, R¹, R², R³, R⁴ and R⁵ as well as n and m have the meanings given under the General Formula I.

The construction of the diazepine ring at the pyrrolo- and pyrazolobenzophenones described is carried out as described in a general manner in Scheme 3.

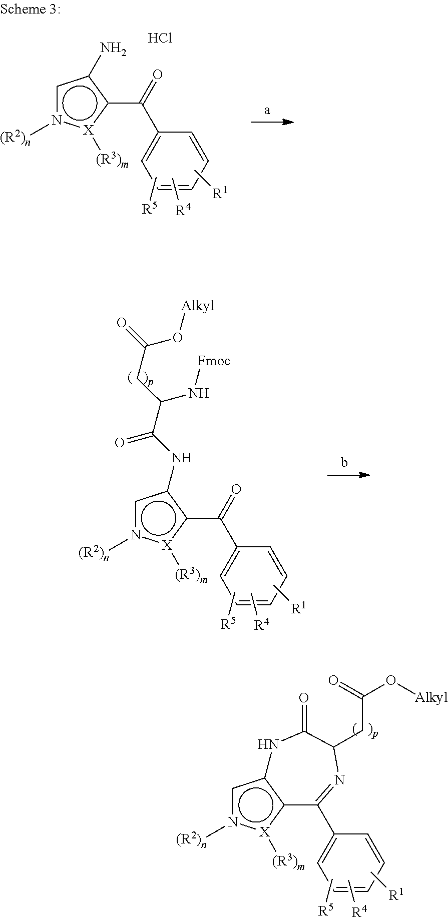

Here, R¹, R², R³, R⁴, R⁵ and X as well as n and m have the meanings given under the General Formula I. In the formulae shown here for the intermediates and for the general formula I, a circle means the presence of possible double bond isomers in the case of X equals nitrogen, as shown below:

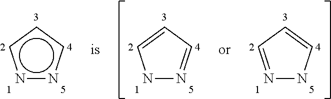

a) e.g. HATU, FMOC-ASP(Oalkyl)-OH; b) e.g. piperidine, RT, then excess HOAc

Here, coupling a) is shown with HATU; however, it may also be effected under other conditions. To this end, a large number of methods compiled in appropriate reference books such as “Compendium of Organic Synthetic Methods”, volume I-VI (Wiley Interscience) or “The Practice of Peptide Synthesis”, Bodansky (Springer Verlag) are available to the person skilled in the art.

Here, use of the Fmoc protective group at the amine is shown. The protective group is removed by addition of a base such as, shown here as an example, piperidine. However, it is also possible to employ other protective groups such as Boc. In this case, a strong acid such as trifluoroacetic acid or hydrochloric acid is employed in Step b).

Subsequently, the triazole ring is constructed as described in Scheme 4.

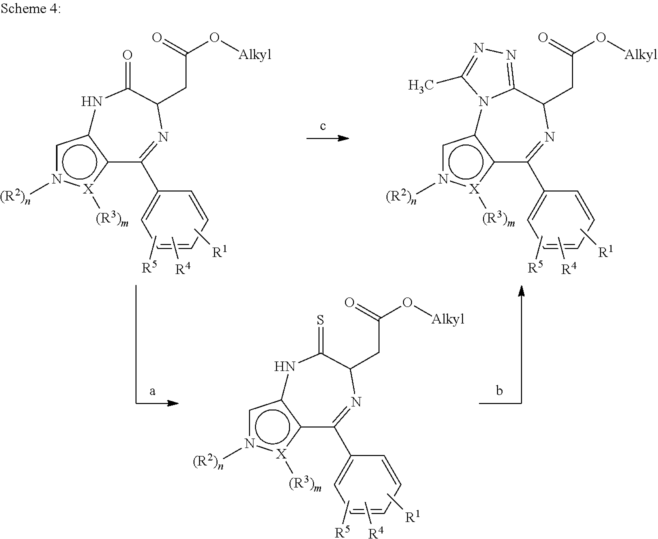

Here, R¹, R², R³, R⁴, R⁵ and X as well as n and m have the meanings given under the General Formula I. In the formulae shown here for the intermediates and for the general formula I, a circle means the presence of possible double bond isomers in the case of X equals nitrogen, as shown below:

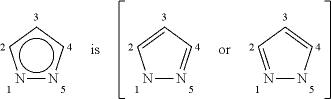

a) Lawesson's reagent, THF, reflux; b) AcNHNH₂, 1-BuOH, reflux; c) NaH, (EtO)₂P(O)Cl or (morpholino)₂P(O)Cl, THF, then AcNHNH₂, 1-BuOH, reflux;

There are further methods for constructing the triazole ring (J. Heterocyclic Chem. (1979), 16, p. 793ff, Moffett et al.; J. Med. Chem. (1980), 23, p. 392ff Hester et al.). Likewise, the reagents and solvents described in Scheme 4 are only mentioned as examples and can be replaced for similar reagents.

The compounds of the general formula I according to the invention where Y is attached via a nitrogen atom are prepared as described in Scheme 5.

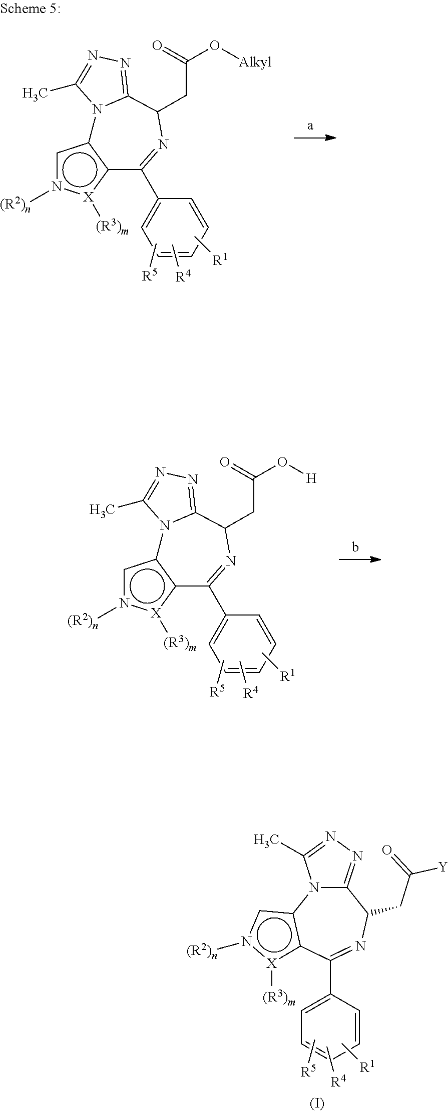

Here, R¹, R², R³, R⁴, R⁵ and X as well as n and m have the meanings given under the General Formula I. In the formulae shown here for the intermediates and for the general formula I, a circle means the presence of possible double bond isomers in the case of X equals nitrogen, as shown below:

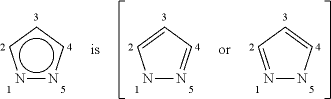

a) NaOH, MeOH, water; or TFA, CH₂Cl₂ b) amine, HATU, base;

According to the nature of the ester, the reaction is effected under basic conditions, or else under acidic conditions.

Alkyl groups preferred in this context are methyl, ethyl or longer homologous esters. The reactions can preferably be performed using alkali metal hydroxides such as lithium hydroxide, sodium hydroxide or potassium hydroxide in aqueous alcoholic solutions. Branched alkyl groups such as tert-butyl esters can preferably be hydrolysed under acidic conditions. The person skilled in the art is aware of a multitude of methods. For illustrative purposes, mention is made here merely of the use, for example, of HCl in organic solvents or pure or dilute trifluoroacetic acid.

The amides of the general formula (I) according to the invention are thus prepared by reacting the carboxylic acids for example with the generally commercially available amines specified in the working examples, with additional activation by a method commonly known to those skilled in the art. Possible methods mentioned here are the use of HATU, HBTU, PyBOB or T3P with addition of a suitable base. The conversion of the carboxylic acids to their amides is described in general terms in reference books such as “Compendium of Organic Synthetic Methods”, volume I-VI (Wiley Interscience) or “The Practice of Peptide Synthesis”, Bodansky (Springer Verlag).

The compounds of the general formula I according to the invention where Y is attached via a nitrogen atom are prepared as described in Scheme 6.

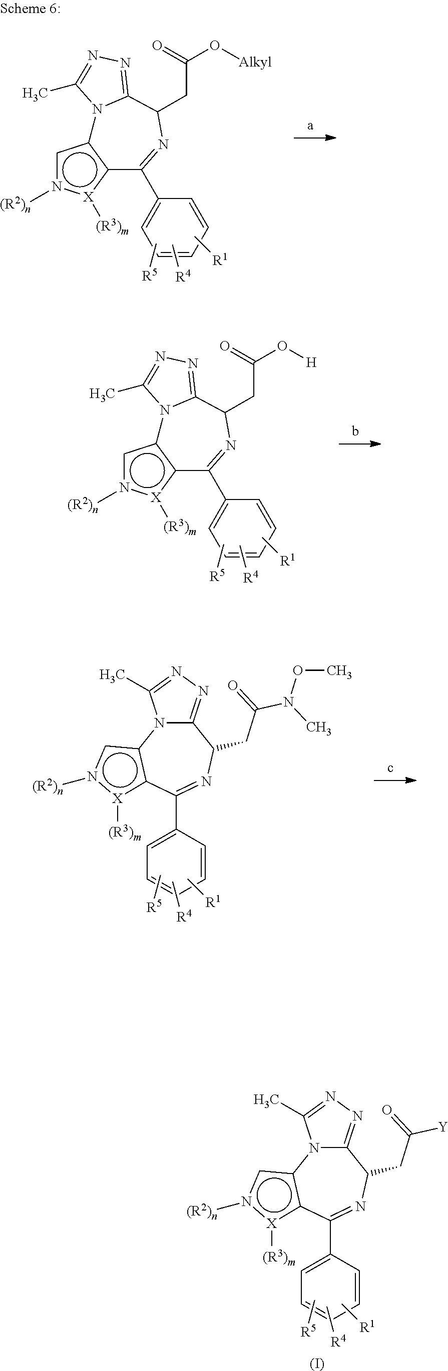

Here, R¹, R², R³, R⁴, R⁵ and X as well as n and m have the meanings given under the General Formula I. In the formulae shown here for the intermediates and for the general formula I, a circle means the presence of possible double bond isomers in the case of X equals nitrogen, as shown below:

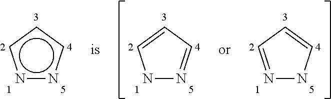

a) NaOH, MeOH, water; or TFA, CH₂Cl₂ b) N,O-dimethylhydroxylamine, HATU, base; c) Y—MgBr, THF,

In the case of such a C attachment, in step b) the coupling to give a Weinreb amide known to the person skilled in the art can be carried out using N,O-dimethylhydroxylamine. In Step c), using, for example, an alkylmagnesium (Grignard) or alkyllithium reagent known to the person skilled in the art, the intermediate is then converted into compounds of the general formula (I). The preparation of such alkylmagnesium or alkyllithium reagents is generally known to the person skilled in the art and can be carried out starting with corresponding alkyl halides such as iodides, bromides or chlorides using, for example, the elemental metal, for example magnesium or lithium, or else by reaction with a corresponding reactive alkylmagnesium or alkyllithium reagent such as diisopropylmagnesium or butyllithium.

### Abbreviations:

Asp aspartic acid
Boc tert-butoxycarbonyl
Boc anhydride di-tert-butyl dicarbonate (CAS 24424-99-5)
CDCl₃ deuterochloroform
CO₂ carbon dioxide
d day
DMF dimethylformamide
DMSO dimethyl sulphoxide
Fmoc fluoren-9-ylmethoxycarbonyl
h hour
HATU (7-aza-1H-benzotriazol-1-yl)-1,1,3,3-tetramethyluronium hexafluorophosphate
HBTU 2-(1H-benzotriazol-1-yl)-1,1,3,3-tetramethyluronium hexafluorophosphate
HPLC high-pressure, high-performance liquid chromatography
KOtBu potassium tert-butoxide
LC-MS liquid chromatography-coupled mass spectrometry
min minutes
NaH sodium hydride
NMR nuclear magnetic resonance spectroscopy
PyBOP benzotriazol-1-yloxytris(dimethylamino)phosphonium hexafluorophosphate
RP-HPLC reversed phase HPLC
RT room temperature
Rₜ retention time (in HPLC)
SFC supercritical fluid chromatography
T3P propylphosphonic anhydride
TFA trifluoroacetic acid
THF tetrahydrofuran

NMR signals are reported with their particular apparent multiplicities or combinations thereof. In this context, s=singlet, d=doublet, t=triplet, q=quartet, qi=quintet, m=multiplet, b=broad signal. Signals having combined multiplicities are reported, for example, as dd=doublet of doublets. For chromatography on silica gel, silica gel having a particle size of 40-63 μm, pre-packed in Biotage (KP-Sil) columns, was usually employed.

### Intermediates for Preparing the Compounds According to the Invention

The examples which follow describe the preparation of the intermediates preferably used for preparing the compounds according to the invention.

### Preparation of the Intermediates Used for Preparing the Compounds According to the Invention

### Intermediate 1A

### 2-{[4-(4-Chlorophenyl)-4-oxobut-2-en-2-yl]amino}acetonitrile

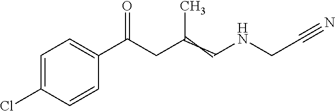 [SMILES: CC(=CNCC#N)CC(=O)c1ccc(Cl)cc1]

29.9 g of sodium bicarbonate were added to a suspension of 32.9 g of 2-aminoacetonitrile hydrochloride (CAS 6011-14-9) in 680 ml of ethanol. After 10 min of stirring at room temperature, 63.9 g of 1-(4-chlorophenyl)butane-1,3-dione (CAS 6302-55-2) and then 300 ml of toluene were added. The mixture was boiled at a Dean-Stark apparatus for 8 hours and conversion was checked by thin-layer chromatography. The mixture was cooled to room temperature, resulting in the formation of a strong precipitate. The mixture was diluted with water and ethyl acetate and extracted three times with ethyl acetate. The combined organic phases were washed with water, dried over sodium sulphate and concentrated under reduced pressure. The residue was subjected to fractional recrystallization from methanol. This gave 66.8 g of the desired 2-{[4-(4-chlorophenyl)-4-oxobut-2-en-2-yl]amino}acetonitrile.

¹H NMR (300 MHz, RT, CDCl₃): δ=2.21 (s, 3H); 4.22 (d, 2H); 5.84 (s, 1H); 7.38 (d, 2H); 7.79 (d, 2H); 11.32 (bs, 1H).

### Intermediate 1B

### 4-Amino-2-methyl-1H-pyrrol-3-yl 4-chlorophenyl ketone hydrochloride

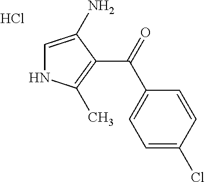 [SMILES: Cl ; Cc1[nH]cc(N)c1C(=O)c1ccc(Cl)cc1]

8.1 g of sodium ethoxide were added to a suspension of 27.3 g of Intermediate 1A in 221 ml of ethanol, and the mixture was then stirred at RT for 30 min. Disappearance of the starting material was monitored by thin-layer chromatography. 63 ml of HCl in dioxane (4 M) were added and the mixture was stirred for 30 min. 500 ml of diethyl ether were then added, the mixture was stirred and the solid was filtered off with suction. This gave 37 g of the desired 4-amino-2-methyl-1H-pyrrol-3-yl 4-chlorophenyl ketone hydrochloride.

¹H NMR (300 MHz, RT, DMSO-d6): δ=1.89 (s, 3H); 6.92 (d, 1H); 7.54 (d, 2H); 7.61 (d, 2H); 9.82 (bs, 2.5H); 11.82 (s, 1H).

### Intermediate 1C

### tert-Butyl N-[4-(4-chlorobenzoyl)-5-methyl-1H-pyrrol-3-yl]carbamate

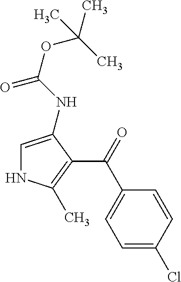 [SMILES: Cc1[nH]cc(NC(=O)OC(C)(C)C)c1C(=O)c1ccc(Cl)cc1]

At 0° C., 14.3 g of sodium carbonate were added to a solution of 36.5 g of Intermediate 1B and 29.4 g of Boc anhydride in 730 ml of dichloromethane. The cooling bath was removed and the mixture was stirred at room temperature for 6 hours. The reaction was monitored by thin-layer chromatography. A further 29.4 g of Boc anhydride and 10 ml of triethylamine were added and the mixture was stirred for one hour. The reaction was added to water and extracted three times with dichloromethane and the extracts were dried over sodium sulphate and concentrated under reduced pressure (1 mbar) on a rotary evaporator. The solution that remained was digested with pentane and the resulting solid was filtered off with suction. This gave 29.1 g of tert-butyl N-[4-(4-chlorobenzoyl)-5-methyl-1H-pyrrol-3-yl]carbamate.

¹H NMR (300 MHz, RT, CDCl₃): δ=1.48 (s, 9H); 1.89 (s, 3H); 7.07 (s, 1H); 7.39 (d, 2H); 7.50 (d, 2H); 8.75 (bs, 1H); 8.9 (bs, 1H).

### Intermediate 1D

### tert-Butyl N-[4-(4-chlorobenzoyl)-1,5-dimethyl-1H-pyrrol-3-yl]carbamate

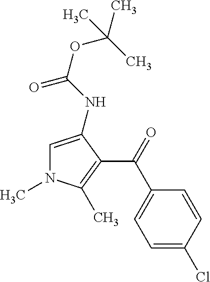 [SMILES: Cc1c(C(=O)c2ccc(Cl)cc2)c(NC(=O)OC(C)(C)C)cn1C]

At RT, 6.84 g of KOtBu were added to a solution of 20 g of Intermediate 1C in 160 ml of THF. The mixture was stirred for 10 min, 3.8 ml of iodomethane were then added dropwise and the mixture was stirred at RT for 4 h. The mixture was added to ice-water and extracted three times with ethyl acetate. The combined organic phases were washed with saturated sodium chloride solution and dried over sodium sulphate, and the solvent was removed under reduced pressure. The residue was taken up in dichloromethane and hexane was added, resulting in the precipitation of the desired product, which was filtered off with suction: 13.5 g. The mother liquor, which contained more product, was purified by chromatography on silica gel, giving a further 3.2 g of the desired tert-butyl N-[4-(4-chlorobenzoyl)-1,5-dimethyl-1H-pyrrol-3-yl]carbamate.

¹H NMR (300 MHz, RT, CDCl₃): δ=1.49 (s, 9H); 1.85 (s, 3H); 3.47 (s, 3H); 7.07 (s, 1H); 7.41 (d, 2H); 7.52 (d, 2H); 8.73 (bs, 1H).

### Intermediate 1E

### 4-Amino-1,2-dimethyl-1H-pyrrol-3-yl 4-chlorophenyl ketone hydrochloride

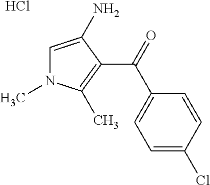 [SMILES: Cl ; Cc1c(C(=O)c2ccc(Cl)cc2)c(N)cn1C]

A solution of 14.6 g of Intermediate 1D in 157 ml of HCl in dioxane solution (4 M) was stirred at room temperature for 4 hours. The solution was stirred into 21 of methyl tert-butyl ether, resulting in the crystallization of the product. Filtration gave 10.1 g of 4-amino-1,2-dimethyl-1H-pyrrol-3-yl 4-chlorophenyl ketone hydrochloride.

¹H NMR (300 MHz, RT, CDCl₃): δ=1.88 (s, 3H); 3.53 (s, 3H); 7.05 (2, 1H); 7.56 (d, 2H); 7.62 (d, 2H); 9.88 (bs, 2H).

### Intermediate 1F

### Methyl 2-(3S)-[5-(4-chlorophenyl)-6,7-dimethyl-2-oxo-1,2,3,7-tetrahydropyrrolo[3,4-e][1,4]diazepin-3-yl]acetate

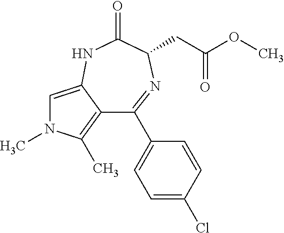

Under argon and at room temperature, a solution of 10.1 g of Intermediate 1E, 13.1 g of Fmoc-1-Asp(OMe)-OH (CAS 145038-53-5), 6.2 ml diisopropylethylamine and 13.5 g of HATU in 144 ml of THF was stirred for 14 h. The mixture was partitioned between water and dichloromethane, the organic phase was removed and the aqueous phase was once more extracted with dichloromethane. The combined organic phases were washed with water and saturated sodium chloride solution, dried over sodium sulphate and concentrated. This gave methyl N-[4-(4-chlorobenzoyl)-1,5-dimethyl-1H-pyrrol-3-yl]-N²-[(9H-fluoren-9-ylmethoxy)carbonyl]-L-alpha-aspartate which was dissolved in 220 ml of THF. 19 g of piperidine were then added and the mixture was stirred at RT for 4.5 h. Subsequently, 76 ml of glacial acetic acid were added and the mixture was stirred for a further 14 h. The mixture was added to water and extracted three times with dichloromethane and the extracts were washed with saturated sodium chloride solution, dried over sodium sulphate and concentrated under reduced pressure. The crude product obtained was purified by chromatography on silica gel (hexane/ethyl acetate gradient). This gave 7.1 g of methyl 2-(3S)-[5-(4-chlorophenyl)-6,7-dimethyl-2-oxo-1,2,3,7-tetrahydropyrrolo[3,4-e][1,4]diazepin-3-yl]acetate.

¹H NMR (300 MHz, RT, CDCl₃): δ=1.79 (s, 3H); 3.12 (dd, 1H); 3.40 (dd, 1H); 3.53 (s, 3H); 3.73 (s, 3H); 4.40 (t, 1H); 6.45 (s, 1H); 7.32 (d, 2H); 7.47 (d, 2H); 7.97 (s, 1H).

### Intermediate 1G

### Methyl 2-(4S)-[6-(4-chlorophenyl)-1,7,8-trimethyl-4,8-dihydropyrrolo[3,4-f][1,2,4]triazolo[4,3-a][1,4]diazepin-4-yl]acetate

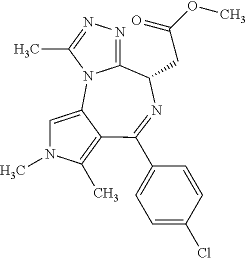

At −78° C. and under argon, 0.9 ml of KOtBu solution (1M in THF) was added to a solution of 300 mg of Intermediate 1F in 2.7 ml of THF. The temperature was increased to −10° C. and stirring was continued for another 30 min. The mixture was cooled again to −78° C. and 173 mg of diethyl chlorophosphate (CAS 814-49-3) were added. Over a period of 30 min, the temperature was increased to −10° C., and stirring was continued for another 2.5 hours. 93 mg of acetylhydrazine were added and the mixture was warmed to RT and stirred for 1 h. After addition of 2.7 ml of butan-1-ol, the mixture was stirred at 85° C. for 4 h. The mixture was concentrated under reduced pressure and purified by chromatography on silica gel (dichloromethane/methanol gradient). This gave 760 mg of a contaminated product which was purified by RP-HPLC (column: C8 Kromasil, mobile phase: methanol/water (0.1% by volume formic acid) gradient). This gave 42 mg of methyl 2-(4S)-[6-(4-chlorophenyl)-1,7,8-trimethyl-4,8-dihydropyrrolo[3,4-f][1,2,4]triazolo[4,3-a][1,4]diazepin-4-yl]acetate.

¹H NMR (300 MHz, RT, CDCl₃): δ=1.86 (s, 3H); 2.60 (s, 3H); 3.64 (d, 2H); 3.68 (s, 3H); 3.80 (s, 3H); 4.76 (t, 1H); 6.8 (s, 1H); 7.35 (d, 2H); 7.47 (d, 2H).

### Intermediate 2A

### 2-[(4-Oxo-4-phenylbut-2-en-2-yl)amino]acetonitrile

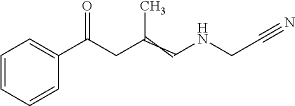 [SMILES: CC(=CNCC#N)CC(=O)c1ccccc1]

17.9 g of sodium bicarbonate were added to a suspension of 19.7 g of 2-aminoacetonitrile hydrochloride (CAS 6011-14-9) in 394 ml of ethanol. After 10 min of stirring at RT, 31.4 g of 1-phenylbutane-1,3-dione (CAS 93-91-4) and then 197 ml of toluene were added. The mixture was boiled at a Dean-Stark apparatus for 20 h and conversion was checked by thin-layer chromatography. The mixture was cooled to room temperature, resulting in the formation of a strong precipitate. The mixture was diluted with water and dichloromethane and extracted three times with dichloromethane. The combined organic phases were washed with water, dried over sodium sulphate and concentrated under reduced pressure. The residue was subjected to fractional recrystallization from methanol. This gave 32.6 g of the desired 2-[(4-oxo-4-phenylbut-2-en-2-yl)amino]acetonitrile.

¹H NMR (300 MHz, RT, CDCl₃): δ=2.20 (s, 3H); 4.22 (d, 2H); 5.89 (s, 1H); 7.39-7.50 (m, 3H); 7.84-7.89 (m, 2H); 11.33 (bs, 1H).

### Intermediate 2B

### 4-Amino-2-methyl-1H-pyrrol-3-yl phenyl ketone hydrochloride

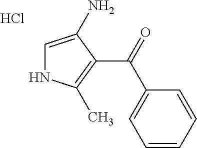

4 g of sodium methoxide were added to a suspension of 11.4 g of Intermediate 2A in 108 ml of ethanol (exothermic), and the mixture was stirred at RT for 15 min. Disappearance of the starting material was monitored by thin-layer chromatography. 28.5 ml of HCl in dioxane (4 M) were added and

the mixture was stirred for 30 min. 110 ml of diethyl ether were then added and the resulting solid was filtered off with suction. This gave 12.8 g of the desired 4-amino-2-methyl-1H-pyrrol-3-yl phenyl ketone hydrochloride.

¹H NMR (300 MHz, RT, DMSO-d6): δ=1.89 (s, 3H); 6.94 (d, 1H); 7.50-7.56 (m, 2H); 7.58-7.64 (m, 3H); 9.81 (bs, 2.5H); 11.74 (s, 1H).

### Intermediate 2C

### tert-Butyl N-(4-benzoyl-5-methyl-1H-pyrrol-3-yl)carbamate

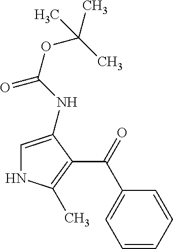 [SMILES: Cc1[nH]cc(NC(=O)OC(C)(C)C)c1C(=O)c1ccccc1]

At 0° C., 3.6 g of sodium carbonate were added to a solution of 7.5 g of Intermediate 2B and 6.9 g of

Boc anhydride in 171 ml of dichloromethane. The mixture was gradually warmed to RT and stirred for 5 h. The mixture was added to water and extracted with dichloromethane and the extracts were washed with water and saturated sodium chloride solution, dried over sodium sulphate and concentrated under reduced pressure. This gave 4.4 g of tert-butyl N-(4-benzoyl-5-methyl-1H-pyrrol-3-yl)carbamate.

¹H NMR (300 MHz, RT, CDCl₃): δ=1.50 (s, 9H); 1.87 (s, 3H); 7.08 (s, 1H); 7.39-7.51 (m, 3H); 7.53-7.59 (m, 2H); 8.16 (bs, 1H); 8.82 (bs, 1H).

### Intermediate 2D

### tert-Butyl N-(4-benzoyl-1,5-dimethyl-1H-pyrrol-3-yl)carbamate

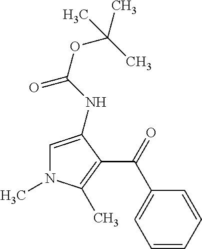 [SMILES: Cc1c(C(=O)c2ccccc2)c(NC(=O)OC(C)(C)C)cn1C]

A solution of 4.4 g of Intermediate 2C, 2.9 ml of dimethyl sulphate and 4.05 g of potassium carbonate in 46 ml of butan-2-one was stirred at 90° C. for 8 h. The mixture was then added to water and extracted three times with dichloromethane and the extracts were washed with water, dried over sodium sulphate and concentrated under reduced pressure. This gave 4.7 g of tert-butyl N-(4-benzoyl-1,5-dimethyl-1H-pyrrol-3-yl)carbamate.

¹H NMR (300 MHz, RT, CDCl₃): δ=1.49 (s, 9H); 1.82 (s, 3H); 3.46 (s, 3H); 7.07 (s, 1H); 7.40-7.46 (m, 2H); 7.47-7.53 (m, 1H); 7.54-7.59 (m, 2H); 8.83 (bs, 1H).

### Intermediate 2E

### 4-Amino-1,2-dimethyl-1H-pyrrol-3-yl phenyl ketone hydrochloride

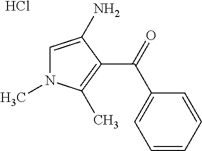 [SMILES: Cl ; Cc1c(C(=O)c2ccccc2)c(N)cn1C]

A solution of 4.2 g of Intermediate 2D in 46.6 ml of HCl in dioxane solution (4 M) was stirred at room temperature for 5 h. The solution was concentrated completely under reduced pressure. This gave 3.4 g of 4-amino-1,2-dimethyl-1H-pyrrol-3-yl phenyl ketone hydrochloride.

¹H NMR (300 MHz, RT, DMSO-d6): δ=1.84 (s, 3H); 3.52 (s, 3H); 7.03 (s, 1H); 7.45-7.54 (m, 2H); 7.55-7.65 (m, 3H); 9.83 (bs, 1H).

### Intermediate 2F

### Methyl 2-(3S)-(6,7-dimethyl-2-oxo-5-phenyl-1,2,3,7-tetrahydropyrrolo[3,4-e][1,4]diazepin-3-yl)acetate

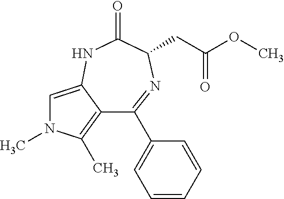

Under argon and at room temperature, a solution of 3.4 g of 2E, 5.01 g of Fmoc-1-Asp(OMe)-OH (CAS 145038-53-5), 4.7 ml triethylamine and 5.16 g of HATU in 52 ml of DMF was stirred for 44 hours. The mixture was partitioned between water and dichloromethane, the organic phase was removed and the aqueous phase was once more extracted with dichloromethane. The combined organic phases were washed with water and saturated sodium chloride solution, dried over sodium sulphate and concentrated. Chromatography on silica gel (hexane/ethyl acetate gradient, then dichloromethane) gave 3.7 g of methyl N-(4-benzoyl-1,5-dimethyl-1H-pyrrol-3-yl)-1-aspartate. These were dissolved in 36 ml of THF, and 0.6 ml of glacial acetic acid was added. The mixture was stirred at room temperature for 5 hours. The mixture was added to water and extracted three times with dichloromethane and the extracts were washed with saturated sodium chloride solution, dried over sodium sulphate and concentrated under reduced pressure. The crude product obtained was purified by chromatography on silica gel (dichloromethane/methanol gradient). This gave 2 g of methyl 2-(3S)-(6,7-dimethyl-2-oxo-5-phenyl-1,2,3,7-tetrahydropyrrolo[3,4-e][1,4]diazepin-3-yl)acetate.

¹H NMR (300 MHz, RT, CDCl₃): δ=1.76 (s, 3H); 3.15 (dd, 1H); 3.42 (dd, 1H); 3.53 (s, 3H); 3.73 (s, 3H); 4.43 (t, 1H); 6.44 (s, 1H); 7.31-7.44 (m, 3H); 7.49-7.55 (m, 2H); 7.73 (s, 1H).

### Intermediate 2G

### Methyl 2-(4S)-(1,7,8-trimethyl-6-phenyl-4,8-dihydropyrrolo[3,4-f][1,2,4]triazolo[4,3-a][1,4]diazepin-4-yl)acetate

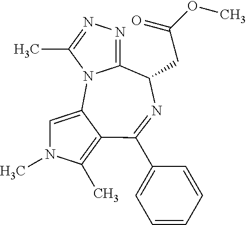

At −78° C. and under argon, 3.3 ml of KOtBu solution (1M in THF) were added to a solution of 1 g of Intermediate 2F in 10 ml of THF. The temperature was increased to −10° C. and stirring was continued for another 30 min. The mixture was cooled again to −78° C. and 637 mg of diethyl chlorophosphate (CAS 814-49-3) were added. Over a period of 30 min, the temperature was increased to −10° C., and stirring was continued for another 2.5 hours. 342 mg of acetylhydrazine were added and the mixture was warmed to RT and stirred for 1 h. After addition of 10 ml of butan-1-ol, the mixture was stirred at 85° C. for 3 h. The mixture was concentrated under reduced pressure and purified by chromatography on silica gel (dichloromethane/methanol gradient). This gave 300 mg of a contaminated product which was purified by RP-HPLC (column: X-Bridge C18 5 μm 100×30 mm, mobile phase: acetonitrile/water (0.1% by volume formic acid) gradient). This gave 75 mg of methyl 2-(4S)-(1,7,8-trimethyl-6-phenyl-4,8-dihydropyrrolo[3,4-f][1,2,4]triazolo[4,3-a][1,4]diazepin-4-yl)acetate.

¹H NMR (300 MHz, RT, CDCl₃): δ=1.80 (s, 3H); 2.57 (s, 3H); 3.63 (d, 2H); 3.65 (s, 3H); 3.77 (s, 3H); 4.75 (t, 1H); 6.77 (s, 1H); 7.30-7.44 (m, 3H); 7.45-7.51 (m, 2H).

### Intermediate 3A

### 1-Thia-6-azaspiro[3.3]heptane 1,1-dioxide, TFA salt

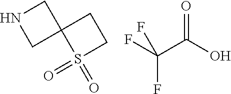

45 ml of TFA were added to 15 g of tert-butyl 1-thia-6-azaspiro[3,3]heptane-6-carboxylate (Org. Lett. 12, (2010), p. 1944-7, Carreira et al.) in 150 ml of dichloromethane, and the mixture was stirred at room temperature overnight. The solvent was removed completely under reduced pressure. This gave 15.05 g of 1-thia-6-azaspiro[3.3]heptane 1,1-dioxide, TFA salt.

¹H NMR (300 MHz, RT, CDCl₃): δ=2.38-2.47 (m, 2H); 4.03-4.12 (m, 2H); 4.30 (d, 2H); 4.37 (d, 2H); 9.32 (bs, 2H).

### Intermediate 4A

### 4-Chlorophenyl 1-methyl-4-nitro-1H-pyrazol-3-yl ketone

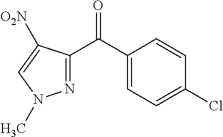 [SMILES: Cn1cc([N+](=O)[O-])c(C(=O)c2ccc(Cl)cc2)n1]

11.45 g of 1-methyl-4-nitro-1H-pyrazole-3-carboxylic acid (CAS 4598-86-1) were added carefully to 52.1 ml of thionyl chloride, and the mixture was then heated at reflux with stirring for 3.5 h. After cooling, the mixture was concentrated under reduced pressure and dried further under oil pump vacuum. This gave 12.75 g of 1-methyl-4-nitro-1H-pyrazole-3-carbonyl chloride which were used for the next step without further purification.

A solution of 12.68 g of the acid chloride prepared beforehand in 200 ml of chlorobenzene was added to a suspension of 8.92 g of aluminium trichloride in 53 ml of chlorobenzene.

Subsequently, the mixture was stirred at 120° C. for 2 h and then at 25° C. for 16 h. The reaction mixture was diluted with 250 ml of ethyl acetate and extracted with 150 ml of water. After phase separation, the aqueous phase was extracted three times with in each case 150 ml of ethyl acetate. The combined organic phases were washed with water and saturated sodium chloride solution, dried over sodium sulphate and concentrated under reduced pressure. The crude product obtained was purified by chromatography on silica gel (hexane/ethyl acetate gradient). This gave 14.7 g of 4-chlorophenyl 1-methyl-4-nitro-1H-pyrazol-3-yl ketone.

¹H NMR (400 MHz, DMSO-d6) δ=3.95 (s, 3Hδ) 7.59-7.64 (m, 2H) 7.85-7.91 (m, 2H) 9.01 (s, 1H).

### Intermediate 4B

### 4-Amino-1-methyl-1H-pyrazol-3-yl 4-chlorophenyl ketone

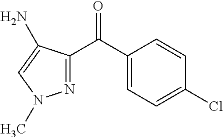 [SMILES: Cn1cc(N)c(C(=O)c2ccc(Cl)cc2)n1]

14.7 g of Intermediate 4A were dissolved in a mixture of 370 ml of ethanol and 185 ml of water, and 30.9 g of iron filings followed by 14.8 g of ammonium chloride were added. Using a mechanical stirrer, the orange-brown suspension was stirred at an oil bath temperature of 90° C. for one hour. After cooling, the reaction mixture was filtered through kieselguhr and the filtrate was concentrated under reduced pressure. The residue obtained in this manner was taken up in ethyl acetate and washed with water. After phase separation, the aqueous phase was extracted with ethyl acetate and the combined organic phases were washed once with water and once with saturated sodium chloride solution. After drying over sodium sulphate, the mixture was concentrated under reduced pressure. The crude product obtained was purified by chromatography on silica gel (hexane/ethyl acetate gradient). This gave 12.6 g of 4-amino-1-methyl-1H-pyrazol-3-yl 4-chlorophenyl ketone.

¹H NMR (400 MHz, DMSO-d6) δ=3.81 (s, 3H) 5.26 (s, 2H) 7.18 (s, 1H) 7.52-7.56 (m, 2H) 8.15-8.20 (m, 2H).

### Intermediate 4C

### Methyl N-[3-(4-chlorobenzoyl)-1-methyl-1H-pyrazol-4-yl]-N²-[(9H-fluoren-9-yl-methoxy) carbonyl]-1-aspartate

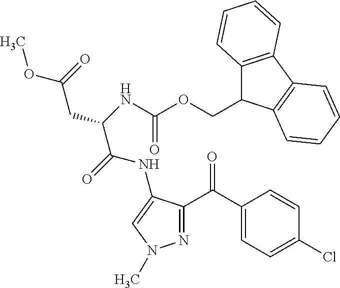 [SMILES: COC(=O)C[C@H](NC(=O)OCC1c2ccccc2-c2ccccc21)C(=O)Nc1cn(C)nc1C(=O)c1ccc(Cl)cc1]

33.4 g of PyBOP and 22.4 ml of N,N-diisopropylethylamine were added to a solution of 11.65 g of Intermediate 4B and 21.9 g of (S)-2-{[(9H-fluoren-9-ylmethoxy)carbonyl]amino}-4-methoxy-4-oxobutanoic acid [Fmoc-L-Asp(OMe)-OH, (CAS 145038-53-5)] in 321 ml of THF. The reaction mixture was stirred at 40° C. for 16 h and, after cooling, concentrated under reduced pressure. The crude product obtained in this manner was combined with an analogous reaction starting with 12.6 g of the title compound from Example 4B which had been stirred at 40° C. for 3 hours and pre-purified by chromatography on silica gel (first hexane/ethyl acetate gradient, then ethyl acetate/methanol gradient with a methanol fraction of up to 25%). The 100 g of crude product obtained in this manner were then purified by chromatography on silica gel (hexane/ethyl acetate gradient). This gave 44.4 g of methyl N-[3-(4-chlorobenzoyl)-1-methyl-1H-pyrazol-4-yl]-N²-[(9H-fluoren-9-ylmethoxy)carbonyl]-L-aspartate which was pure enough for further reactions.

¹H NMR (400 MHz, DMSO-d6) δ=2.70 (dd, 1H), 2.90 (dd, 1H), 3.58 (s, 3H), 3.95 (s, 3H), 4.22-4.45 (m, 3H), 4.56 (q, 1H), 7.25 (t, 2H), 7.36 (t, 2H), 7.55 (d, 2H), 7.71 (d, 2H), 7.85 (d, 2H), 8.11-8.22 (m, 3H), 8.37 (s, 1H), 10.29 (s, 1H).

### Intermediate 4D

### Methyl 2-[(6S)-8-(4-chlorophenyl)-2-methyl-5-oxo-2,4,5,6-tetrahydropyrazolo[4,3-e][1,4]diazepin-6-yl]acetate

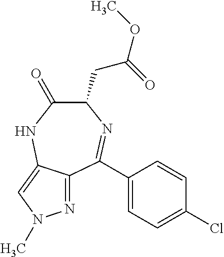 [SMILES: COC(=O)C[C@@H]1N=C(c2ccc(Cl)cc2)c2nn(C)cc2NC1=O]

At 25° C., 5.43 ml of piperidine were quickly added dropwise to a solution of 6.45 g of intermediate 4C in 84 ml of THF, and the mixture was then stirred at this temperature for one hour. The reaction was checked by UPLC-MS, showing complete conversion into the intermediate methyl N-[3-(4-chlorobenzoyl)-1-methyl-1H-pyrazol-4-yl]-L-aspartate. 5 ml of acetic acid were then added dropwise, and the reaction mixture was stirred at 25° C. for a further three hours. A further 2 ml of acetic acid were added, followed, after two hours of stirring, by the addition of another 1 ml of acetic acid. After 16 h of stirring at 25° C., the reaction mixture was then diluted with ethyl acetate and the organic phase was washed with water. After phase separation, the aqueous phase was extracted once with ethyl acetate and the combined organic phases were then washed once with water and once with saturated sodium chloride solution. After drying over sodium sulphate, the mixture was concentrated under reduced pressure. The crude product obtained in this manner was purified by chromatography on silica gel (first hexane/ethyl acetate, then ethyl acetate/methanol gradient with a methanol fraction of up to 50%). This gave 2.87 g of methyl 2-[(6S)-8-(4-chlorophenyl)-2-methyl-5-oxo-2,4,5,6-tetrahydropyrazolo[4,3-e][1,4]diazepin-6-yl]acetate.

¹H NMR (400 MHz, DMSO-d6) δ=2.99 (dd, 1H), 3.18 (dd, 1H), 3.57 (s, 3H), 3.93 (s, 3H), 4.09 (t, 1H), 7.42-7.51 (m, 2H), 7.75 (s, 1H), 7.78-7.85 (m, 2H), 10.43 (s, 1H).

### Intermediate 4E

### Methyl 2-[(4S)-6-(4-chlorophenyl)-1,8-dimethyl-4,8-dihydropyrazolo[3,4-f][1,2,4]triazolo[4,3-a][1,4]diazepin-4-yl]acetate

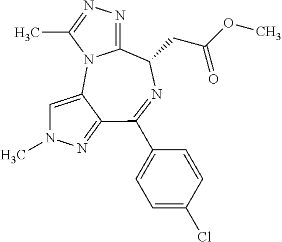

Under argon and at −70° C., 364 mg of NaH (60% suspension in mineral oil) were added carefully to a solution of 2.87 g of Intermediate 4D in 27 ml of THF. The reaction mixture was slowly warmed to 0° C. and, after 20 min, cooled back to −70° C. 1.71 g of diethyl chlorophosphate (CAS 814-49-3) were then added and the mixture was warmed back to 0° C. over a period of 30 min. After a further 30 min of stirring, 1.38 g of acetylhydrazine were added and the reaction mixture was then warmed to 25° C. and stirred for another hour. 27 ml of butanol were then added and the mixture was heated at 85° C. for 2 h. The reaction mixture was added to a little aqueous sodium bicarbonate solution and extracted three times with methylene chloride. The combined organic phases were washed once with saturated sodium chloride solution, dried over sodium sulphate and concentrated under reduced pressure. The crude product obtained in this manner was purified by chromatography on silica gel (first hexane/ethyl acetate, then ethyl acetate/methanol gradient with a methanol fraction of up to 75%). This gave 277 mg of still contaminated product which was purified in two portions by HPLC chromatography (column: Chromatorex RP C-18 10 μm; 125*30 mm, flow rate 60.00 ml/min, acetonitrile/water/formic acid 15:85:0.1, after 9 minutes acetonitrile/water/formic acid 55:45:0.1 (v/v/v)). The combined product fractions were concentrated under reduced pressure, dissolved in 25 ml of ethyl acetate and washed twice with in each case 15 ml of saturated sodium bicarbonate solution. Drying over sodium sulphate and concentration under reduced pressure gave 132 mg of methyl 2-[(4S)-6-(4-chlorophenyl)-1,8-dimethyl-4,8-dihydropyrazolo[3,4-f][1,2,4]triazolo[4,3-a][1,4]diazepin-4-yl]acetate. Analytical HPLC on a chiral carrier material showed that the substance had an ee of 80% (HPLC: Chiralpak IC 3 μm 100×4.6 mm, flow rate 1.0 ml/min, ethanol/methanol/diethylamine 50:50:0.1 (v/v/v).

¹H NMR (400 MHz, DMSO-d6) δ=2.49 (s, 3H), 3.35-3.47 (m, 2H), 3.63 (s, 3H), 4.02 (s, 3H), 4.62 (t, 1H), 7.43-7.48 (m, 2H), 7.67-7.72 (m, 2H), 8.57 (s, 1H).

### Intermediate 4F

### 2-[(4S)-6-(4-Chlorophenyl)-1,8-dimethyl-4,8-dihydropyrazolo[3,4-f][1,2,4]triazolo[4,3-a][1,4]diazepin-4-yl]acetic acid, sodium salt

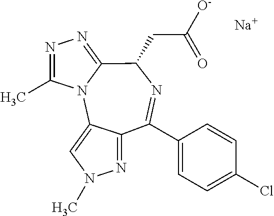 [SMILES: Cc1nnc2n1-c1cn(C)nc1C(c1ccc(Cl)cc1)=N[C@H]2CC(=O)[O-] ; [Na+]]

0.38 ml of an aqueous 1N sodium hydroxide solution was added dropwise to a solution of 132 mg of Intermediate 4E in 2.6 ml of methanol. After 1 h and after 3 h of stirring at 25° C., in each case 0.3 ml of water was added. After 4 h of stirring at 25° C. in total, the reaction mixture was concentrated under reduced pressure. After addition of toluene, the mixture was once more concentrated under reduced pressure, and this procedure was repeated a further four times and the product was then dried under oil pump vacuum for 1 h.

This gave 145 mg of the title compound. Without further purification, this crude product was used for the amide formation.

### Intermediate 5A

### 4-Chlorophenyl 1-methyl-4-nitro-1H-pyrazol-5-yl ketone

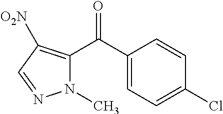 [SMILES: Cn1ncc([N+](=O)[O-])c1C(=O)c1ccc(Cl)cc1]

Analogously to the preparation of Intermediate 4A, 14.4 g of 1-methyl-4-nitro-1H-pyrazole-5-carbonyl chloride (CAS 1006962-20-4) and 320 ml of chlorobenzene gave 2.43 g of 4-chlorophenyl 1-methyl-4-nitro-1H-pyrazol-5-yl ketone as a solid in still contaminated form.

¹H NMR (400 MHz, DMSO-d6)=3.80 (s, 3H), 7.62-7.66 (m, 2H), 7.86-7.90 (m, 2H), 8.43 (s, 1H) (characteristic signals of the main component).

### Intermediate 5B

### 4-Amino-1-methyl-1H-pyrazol-5-yl 4-chlorophenyl ketone

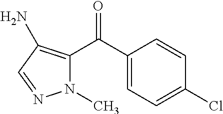 [SMILES: Cn1ncc(N)c1C(=O)c1ccc(Cl)cc1]

Analogously to the preparation of Intermediate 4B, 2.42 g of Intermediate 5A gave 1.11 g of 4-amino-1-methyl-1H-pyrazol-5-yl 4-chlorophenyl ketone as a solid in still contaminated form.

¹H NMR (400 MHz, DMSO-d6)=3.44 (s, 2H), 4.69 (s, 3H), 7.04 (s, 2H), 7.54-7.59 (m, 2H), 7.63-7.68 (m, 2H) (characteristic signals of the main component).

### Intermediate 5C

### Methyl N-[5-(4-chlorobenzoyl)-1-methyl-1H-pyrazol-4-yl]-N²-[(9H-fluoren-9-yl-methoxy)carbonyl]-1-aspartate

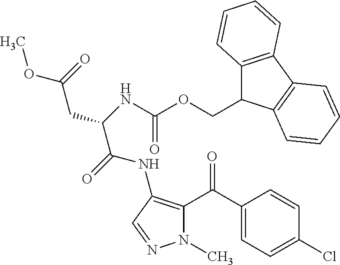 [SMILES: COC(=O)C[C@H](NC(=O)OCC1c2ccccc2-c2ccccc21)C(=O)Nc1cnn(C)c1C(=O)c1ccc(Cl)cc1]

Analogously to the preparation of Intermediate 4C, 1.10 g of Intermediate 5B and 2.08 g of (5)-2-{[(9H-fluoren-9-ylmethoxy)carbonyl]amino}-4-methoxy-4-oxobutanoic acid [Fmoc-L-Asp(OMe)-OH, (CAS 145038-53-5)] gave 3.18 g of methyl N-[5-(4-chlorobenzoyl)-1-methyl-1H-pyrazol-4-yl]-N²-[(9H-fluoren-9-yl-methoxy)carbonyl]-L-aspartate in still contaminated form.

¹H NMR (300 MHz, DMSO-d6): δ [ppm]=2.13-2.20 (m, 2H), 3.52 (s, 3H), 3.81 (s, 3H), 4.11-4.27 (m, 4H), 7.20-7.69 (m, 12H), 7.85 (d, 2H), 9.85 (s, 1H) (characteristic signals of the main component)).

### Intermediate 5D

### Methyl 2-[(6S)-8-(4-chlorophenyl)-1-methyl-5-oxo-1,4,5,6-tetrahydropyrazolo[4,3-e][1,4]diazepin-6-yl]acetate

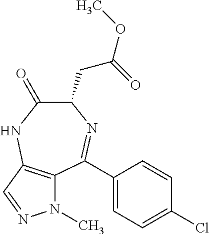 [SMILES: COC(=O)C[C@@H]1N=C(c2ccc(Cl)cc2)c2c(cnn2C)NC1=O]

Analogously to the preparation of Intermediate 4D, 3.18 g of the compound prepared in 5C gave 1.20 g of methyl 2-[(6S)-8-(4-chlorophenyl)-1-methyl-5-oxo-1,4,5,6-tetrahydropyrazolo[4,3-e][1,4]diazepin-6-yl]acetate as a solid in still contaminated form.

¹H NMR (400 MHz, DMSO-d6) δ=3.01 (d, 1H), 3.16-3.27 (m, 1H), 3.39 (s, 3H), 3.58 (s, 3H), 3.94-4.08 (m, 1H), 7.42-7.49 (m, 3H), 7.51-7.56 (m, 2H), 10.66 (s, 1H).

### Intermediate 5E

### Methyl 2-[4S)-6-(4-chlorophenyl)-1,7-dimethyl-4,7-dihydropyrazolo[3,4-f][1,2,4]triazolo[4,3-a][1,4]diazepin-4-yl]acetate

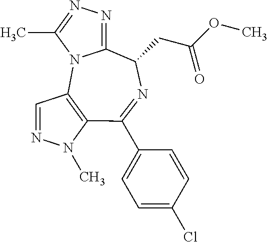

At −70° C., 425 mg of potassium tert-butoxide and, after 30 min of stirring, at −10° C., 714 mg of diethyl chlorophosphate (CAS 814-49-3) were added to a solution of 1.20 g of Intermediate 5D in 11.2 ml of THF. After one hour of stirring at −10° C., 574 mg of acetylhydrazine were added and the mixture was stirred at 25° C. for one hour. This was followed by addition of 11.2 ml of butanol and heating at 110° C. for two hours.

After cooling, the reaction mixture was diluted with dichloromethane and washed in each case once with 10 ml of saturated sodium bicarbonate solution and saturated sodium chloride solution. After drying over sodium sulphate, the mixture was concentrated under reduced pressure. The crude product obtained was purified by chromatography on silica gel (hexane/ethyl acetate gradient). This gave 150 mg of methyl 2-[(4S)-6-(4-chlorophenyl)-1,7-dimethyl-4,7-dihydropyrazolo[3,4-f][1,2,4]triazolo[4,3-a][1,4]diazepin-4-yl]acetate.

¹H NMR (400 MHz, DMSO-d6) δ=3.37 (dd, 1H), 3.46 (s, 3H), 3.52 (dd, 1H), 3.63 (s, 3H), 4.59 (t, 1H), 7.40-7.46 (m, 2H), 7.50-7.56 (m, 2H), 8.24 (s, 1H).

### Intermediate 5F

### 2-[(4S)-6-(4-Chlorophenyl)-1,7-dimethyl-4,7-dihydropyrazolo[3,4-f][1,2,4]triazolo[4,3-a][1,4]diazepin-4-yl]acetic acid, sodium salt

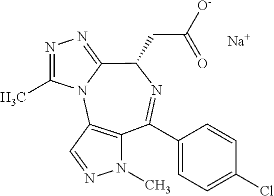 [SMILES: Cc1nnc2n1-c1cnn(C)c1C(c1ccc(Cl)cc1)=N[C@H]2CC(=O)[O-] ; [Na+]]

Analogously to the preparation of Intermediate 4F, but without addition of additional water, 145 mg of Intermediate 5E gave 148 mg of the title compound as a solid. Without further purification, this crude product was used for the amide formation.

### Intermediate 6A

### 2-[(4S)-6-(4-Chlorophenyl)-1,7,8-trimethyl-4,8-dihydropyrrolo[3,4-f][1,2,4]triazolo[4,3-a][1,4]diazepin-4-yl]acetic acid, sodium salt

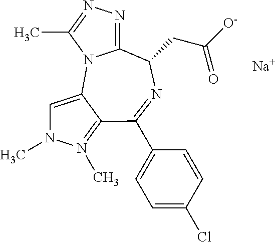

A solution of 550 mg of Intermediate 1G, 1.5 ml of aqueous sodium hydroxide solution (1N) in 2.5 ml of methanol was stirred at RT for 14 hours. The solvent was removed completely under reduced pressure, giving 587 mg of 2-[(4S)-6-(4-chlorophenyl)-1,7,8-trimethyl-4,8-dihydropyrrolo[3,4-f][1,2,4]triazolo[4,3-a][1,4]diazepin-4-yl]acetic acid, sodium salt. The substance was used for the next step without further purification.

Rₜ=0.69 min.

UPLC-MS: Instrument: Waters Acquity UPLC-MS SQD; column: Acquity UPLC BEH C18 1.7 50×2.1 mm; mobile phase A: water+0.1% by volume formic acid (99%), mobile phase B: acetonitrile; gradient: 0-1.6 min 1-99% B, 1.6-2.0 min 99% B; flow rate 0.8 ml/min; temperature: 60° C.; injection: 2 μl; DAD scan: 210-400 nm

The compounds mentioned in the examples above are useful selected intermediates which are preferably used for preparing the compounds according to the invention.

The present invention therefore also provides intermediates of the general formulae Ia and Ib

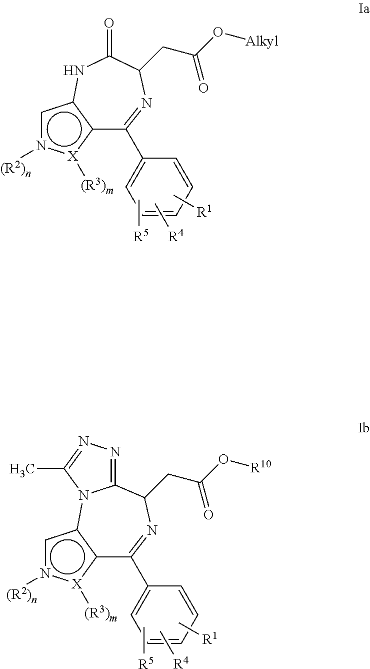

in which R¹, R², R³, R⁴, R⁵, X, n and m have the meanings given in the general formula I and R¹⁰ in the general formula Ib represents hydrogen, and their sodium, potassium, lithium or caesium salts.

The present invention provides in particular the following intermediates:
methyl 2-(4S)-[6-(4-chlorophenyl)-1,7,8-trimethyl-4,8-dihydropyrrolo[3,4][1,2,4]triazolo[4,3-a][1,4]diazepin-4-yl]acetate;
methyl 2-(4S)-(1,7,8-trimethyl-6-phenyl-4,8-dihydropyrrolo[3,4-f][1,2,4]triazolo[4,3-a][1,4]diazepin-4-yl)acetate;
methyl 2-[(4S)-6-(4-chlorophenyl)-1,8-dimethyl-4,8-dihydropyrazolo[3,4-f][1,2,4]triazolo[4,3-a][1,4]diazepin-4-yl]acetate;
2-[(4S)-6-(4-chlorophenyl)-1,8-dimethyl-4,8-dihydropyrazolo[3,4-f][1,2,4]triazolo[4,3-a][1,4]diazepin-4-yl]acetic acid, sodium salt;
methyl 2-[(4S)-6-(4-chlorophenyl)-1,7-dimethyl-4,7-dihydropyrazolo[3,4-f][1,2,4]triazolo[4,3-a][1,4]diazepin-4-yl]acetate;
2-[(4S)-6-(4-chlorophenyl)-1,7-dimethyl-4,7-dihydropyrazolo[3,4-f][1,2,4]triazolo[4,3-a][1,4]diazepin-4-yl]acetic acid, sodium salt;
   and
2-[(4S)-6-(4-chlorophenyl)-1,7,8-trimethyl-4,8-dihydropyrrolo[3,4-f][1,2,4]triazolo[4,3-a][1,4]diazepin-4-yl]acetate, sodium salt.

### Preparation of the Compounds According to the Invention

### EXAMPLES

### Example 1

### (−)-2-[(4S)-6-(4-Chlorophenyl)-1,7,8-trimethyl-4,8-dihydropyrrolo[3,4-f][1,2,4]triazolo-[4,3-a][1,4]diazepin-4-yl]-1-(2-oxa-6-azaspiro[3.3]hept-6-yl)ethan-1-one

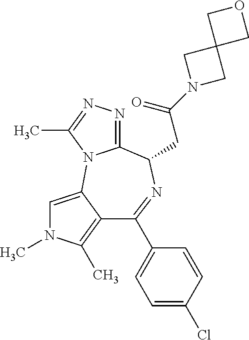 [SMILES: Cc1c2c(cn1C)-n1c(C)nnc1[C@H](CC(=O)N1CC3(COC3)C1)N=C2c1ccc(Cl)cc1]

A solution of 40 mg of Intermediate 1G and 0.13 ml of aqueous sodium hydroxide solution (1M) in 0.2 ml of methanol was stirred at room temperature for 6 h. The mixture was concentrated under reduced pressure and then dissolved in 0.45 ml of DMF. 0.062 ml of triethylamine, 63.5 mg of HATU and 2-Oxa-6-azaspiro[3.3]heptane hemioxalate (CAS 1045709-32-7) were added and the mixture was stirred at RT overnight. The mixture was added to saturated sodium chloride solution/water and extracted three times with ethyl acetate and the extracts were washed twice with saturated sodium chloride solution, dried over sodium sulphate and concentrated under reduced pressure. This gave 45 mg of crude product which was purified by chromatography on modified silica gel (column: Biotage KP-NH 12+M, mobile phase: dichloromethane/methanol gradient). This gave 26 mg of 2-[6-(4-chlorophenyl)-1,7,8-trimethyl-4,8-dihydropyrrolo[3,4-f][1,2,4]triazolo[4,3-a][1,4]diazepin-4-yl]-1-(2-oxa-6-azaspiro[3.3]hept-6-yl)ethan-1-one.

¹H NMR (300 MHz, RT, CDCl₃): δ=1.8 (s, 3H); 2.55 (s, 3H); 3.24 (dd, 1H); 3.35 (dd, 1H); 3.63 (s, 3H); 4.2 (s, 2H); 4.55 (d, 1H); 4.71-4.91 (m, 6H); 6.74 (s, 1H); 7.31 (d, 2H); 7.41 (d, 2H).

Optical rotation: [α_{D}]=−15.9° (chloroform, c=1 g/100 ml).

### Example 2

### 2-(1,7,8-Trimethyl-6-phenyl-4,8-dihydropyrrolo[3,4-f][1,2,4]triazolo[4,3-a][1,4]diazepin-4-yl)-1-(2-oxa-6-azaspiro[3.3]hept-6-yl)ethan-1-one

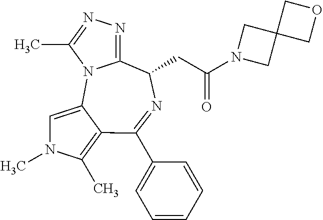 [SMILES: Cc1c2c(cn1C)-n1c(C)nnc1[C@H](CC(=O)N1CC3(COC3)C1)N=C2c1ccccc1]

A solution of 70 mg of Intermediate 2G and 0.13 ml of aqueous sodium hydroxide solution (1M) in 0.2 ml of methanol was stirred at room temperature for 6 h. The mixture was concentrated under reduced pressure and then dissolved in 0.78 ml of DMF. 0.11 ml of triethylamine, 109 mg of HATU and 2-Oxa-6-azaspiro[3.3]heptane hemioxalate (CAS 1045709-32-7) were added and the mixture was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. This gave 85 mg of crude product which was purified by HPLC chromatography (column: X-Bridge C18 5 μm 100×30 mm, mobile phase: acetonitrile/water (0.1% by volume formic acid) gradient). This gave 10 mg of 2-(1,7,8-trimethyl-6-phenyl-4,8-dihydropyrrolo[3,4-f][1,2,4]triazolo[4,3-a][1,4]diazepin-4-yl)-1-(2-oxa-6-azaspiro[3.3]hept-6-yl)ethan-1-one.

¹H NMR (300 MHz, RT, CDCl₃): δ=1.77 (s, 3H); 2.56 (s, 3H); 3.22-3.39 (m, 2H); 4.16-4.27 (m, 2H); 4.60 (d, 1H); 4.74 (d, 1H); 4.75-4.92 (m, 5H); 6.74 (s, 1H); 7.29-7.49 (m, 5H).

### Example 3

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(1,1-dioxo-1λ⁶-1-thia-6-azaspiro[3.3]hept-6-yl)ethan-1-one

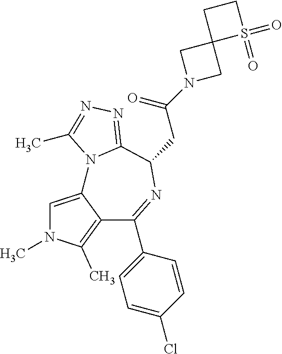 [SMILES: Cc1c2c(cn1C)-n1c(C)nnc1[C@H](CC(=O)N1CC3(CCS3(=O)=O)C1)N=C2c1ccc(Cl)cc1]

A solution of 1.4 g of Intermediate 1G and 2.3 ml of aqueous sodium hydroxide solution (1M) in 14 ml of methanol was stirred at room temperature for 72 h and under reflux for 7 h. The mixture was concentrated under reduced pressure and then dissolved in 19 ml of DMF. 14 ml of triethylamine, 2 mg of HATU and 567 mg of Intermediate 3A were added and the mixture was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 830 mg of (−)-2-[(4S)-6-(4-chlorophenyl)-1,7,8-trimethyl-4,8-dihydro-pyrrolo[3,4-f][1,2,4]triazolo[4,3-a][1,4]diazepin-4-yl]-1-(1,1-dioxo-1λ⁶-thia-6-azaspiro[3.3]hept-6-yl)ethan-1-one.

¹H NMR (300 MHz, RT, CDCl₃): δ=1.78-1.84 (m, 3H); 3.04-3.20 (m, 2H); 3.66 (s, 3H); 4.10-4.35 (m, 5H); 4.47-4.56 (m, 1H); 4.65 (dd, 1H); 4.78 (d, 1H); 7.40-7.51 (m, 5H).

Optical rotation: [α_{D}]=−16.5° (chloroform, c=1 g/100 ml).

### Example 4

### 2-[(4S)-6-(4-Chlorophenyl)-1,8-dimethyl-4,8-dihydropyrazolo[3,4-f][1,2,4]triazolo[4,3-a][1,4]diazepin-4-yl]-1-(2-oxa-6-azaspiro[3.3]hept-6-yl)ethan-1-one

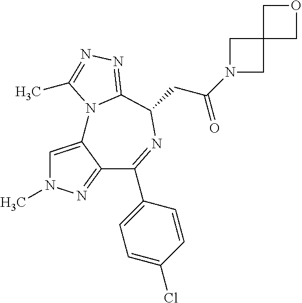 [SMILES: Cc1nnc2n1-c1cn(C)nc1C(c1ccc(Cl)cc1)=N[C@H]2CC(=O)N1CC2(COC2)C1]

195 mg of HATU, 190 μl of triethylamine and 98.4 mg of 2-oxa-6-azaspiro[3.3]heptane hemioxalate (CAS 1045709-32-7) were added in succession to a solution of 134 mg of Intermediate 4F in 1.5 ml of DMF, and the mixture was stirred under nitrogen at 25° C. for 48 h.

The reaction mixture was then diluted with ethyl acetate and the organic phase was washed with water. The aqueous phase was, after phase separation, extracted twice with in each case 25 ml of ethyl acetate. The combined organic phases were washed once with saturated sodium bicarbonate solution and once with water. After drying over sodium sulphate, the mixture was concentrated under reduced pressure. Since purification did not lead to the desired product, the aqueous phase was concentrated and triturated with isopropanol. After filtration, the filtrate was concentrated under reduced pressure and this crude product was then purified by chromatography on silica gel (first hexane/ethyl acetate gradient, then ethyl acetate/methanol with a methanol fraction of up to 100%). This gave 32.1 mg of the title compound as a white solid.

¹H NMR (400 MHz, DMSO-d6) δ=2.46-2.48 (3H under DMSO signal), 3.12 (d, 2H), 3.97-4.09 (m, 5H), 4.47 (d, 2H), 4.56 (t, 1H), 4.63-4.73 (m, 4H), 7.42-7.49 (m, 2H), 7.64-7.70 (m, 2H), 8.55 (s, 1H).

### Example 5

### 2-[(4S)-6-(4-Chlorophenyl)-1,7-dimethyl-4,7-dihydropyrazolo[3,4-f][1,2,4]triazolo[4,3-a][1,4]diazepin-4-yl]-1-(2-oxa-6-azaspiro[3.3]hept-6-yl)ethan-1-one

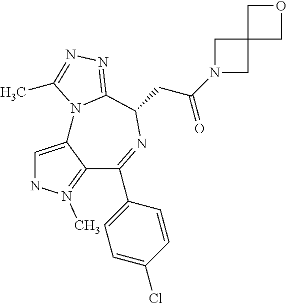

Analogously to the preparation of Example 4, 148 mg of Intermediate 5F and 107 mg of 2-oxa-6-azaspiro[3.3]heptane hemioxalate (CAS 1045709-32-7) (in deviation, the reaction mixture, after the reaction had gone to completion, concentrated under reduced pressure and the resulting crude product was purified by chromatography on silica gel (twice)) gave 109 mg of the title compound as a solid.

¹H-NMR (300 MHz, DMSO-d6): δ=2.54 (s, 3H), 2.99-3.11 (m, 1H), 3.20-3.28 (m, 1H, partially under H2O signal), 3.45 (s, 3H), 4.00-4.08 (m, 2H), 4.40-4.60 (m, 3H), 4.64-4.73 (m, 4H), 7.38-7.44 (m, 2H), 7.50-7.56 (m, 2H), 8.22 (s, 1H).

### Example 6

### (−)-8-{2-[(4S)-6-(4-Chlorophenyl)-1,7,8-trimethyl-4,8-dihydropyrrolo[3,4-f][1,2,4]triazolo[4,3-a][1,4]diazepin-4-yl]acetyl}-8-azabicyclo[3.2.1]octan-3-one

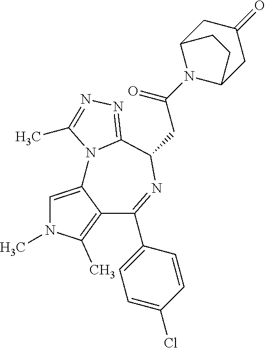 [SMILES: Cc1c2c(cn1C)-n1c(C)nnc1[C@H](CC(=O)N1C3CCC1CC(=O)C3)N=C2c1ccc(Cl)cc1]

A solution of 73 mg of Intermediate 6A, 0.1 ml of triethylamine, 102.5 mg of HATU and 25 mg of nortropinone hydrochloride (CAS 25602-68-0) in 0.97 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 57 mg of (−)-8-{2-[(4S)-6-(4-chlorophenyl)-1,7,8-trimethyl-4,8-dihydropyrrolo[3,4-f][1,2,4]triazolo[4,3-a][1,4]diazepin-4-yl]acetyl}-8-azabicyclo[3.2.1]octan-3-one.

¹H NMR (300 MHz, RT, DMSO-d6): δ=1.50-1.67 (m), 1.76 (d, 3H); 1.85-2.00 (m), 2.03-2.35 (m), 3.63 (s, 3H); 4.61-4.69 (m, 2H); 4.78-4.88/m, 1H); 7.34-7.47 (3S, 5H).

Optical rotation: [α_{D}]=−40.4° (methanol, c=1 g/100 ml).

### Example 7

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-{(1S,4S)-2-oxa-5-azabicyclo[2.2.1]hept-5-yl}ethan-1-one

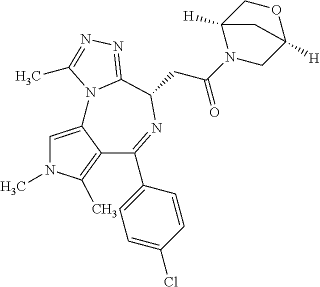 [SMILES: Cc1c2c(cn1C)-n1c(C)nnc1[C@H](CC(=O)N1C[C@H]3C[C@@H]1CO3)N=C2c1ccc(Cl)cc1]

A solution of 73 mg of Intermediate 6A, 0.1 ml of triethylamine, 102.5 mg of HATU and 25 mg of (1S,4S)-2-oxa-5-azabicyclo[2.2.1]heptane (CAS 31560-06-2) in 0.97 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by RP-HPLC chromatography (C18 5 μm 100×30 mm, mobile phase water/acetonitrile gradient, 0.1% formic acid added, flow rate: 50 ml/min) The resulting substance was dissolved in dichloromethane and extracted with sodium bisulphate solution and saturated sodium chloride solution. The solution was dried with sodium sulphate and concentrated under reduced pressure. This gave 23 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-{(1S,4S)2-oxa-5-azabicyclo[2.2.1]hept-5-yl}ethan-1-one.

¹H NMR (300 MHz, RT, DMSO-d6): δ=1.77 (s, 3H); 3.20 (bs, 1H); 3.22 (ddd, 1H); 3.52-3.79 (m+s, 6H); 3.79 (dd, 1H); 4.59 (q, 1H); 4.61 (d, 1H); 4.83 (d, 1H); 7.40 (s, 1H); 7.43 (s, 4H).

Optical rotation: [α_{D}]=−22.9° (chloroform, c=1 g/100 ml).

### Example 8

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(8-oxa-3-azabicyclo[3.2.1]oct-3-yl) ethan-1-one

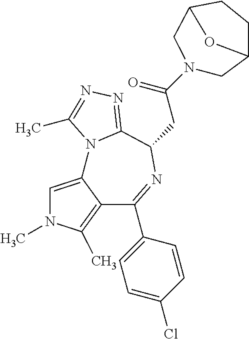 [SMILES: Cc1c2c(cn1C)-n1c(C)nnc1[C@H](CC(=O)N1CC3CCC(C1)O3)N=C2c1ccc(Cl)cc1]

A solution of 73 mg of Intermediate 6A, 0.1 ml of triethylamine, 102.5 mg of HATU and 29.6 mg of 8-oxa-3-azabicyclo[3.2.1]octane hydrochloride (CAS 54745-74-3) in 0.97 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by RP-HPLC chromatography (XBridge C18 5 μm 100×30 mm, mobile phase water/acetonitrile gradient, 0.1% formic acid added, flow rate 50 ml/min) The resulting substance was dissolved in dichloromethane and extracted with sodium bisulphate solution and saturated sodium chloride solution. The solution was dried with sodium sulphate and concentrated under reduced pressure. This gave 24 mg of (−)-2-[(4S)-6-(4-chlorophenyl)-1,7,8-trimethyl-4,8-dihydro-pyrrolo[3,4-f][1,2,4]triazolo[4,3-a][1,4]diazepin-4-yl]-1-(2-oxa-5-azabicyclo[2.2.1]hept-5-yl)ethan-1-one.

¹H NMR (300 MHz, RT, DMSO-d6): δ=1.42-1.59 (m, 1H); 1.70-1.85 (m+s, 5H); 2.27 (t, 1H); 3.03-3.15+3.3-3.5 (2m, 3H); 3.56-3.70 (m+s, 4H); 3.83 (t, 1H); 3.94 (t, 1H); 4.30 (bs, 2H); 4.63 (q, 1H); 7.38-7.48 (s+m, 5H).

Optical rotation: [α_{D}]=−18.3° (chloroform, c=1 g/100 ml).

### Example 9

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(1-oxa-4-azaspiro[5.5]undec-4-yl)ethanone

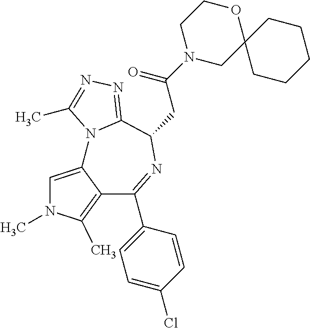 [SMILES: Cc1c2c(cn1C)-n1c(C)nnc1[C@H](CC(=O)N1CCOC3(CCCCC3)C1)N=C2c1ccc(Cl)cc1]

A solution of 400 mg of Intermediate 6A, 0.58 ml of triethylamine, 594 mg of HATU and 220 mg of 1-oxa-4-azaspiro[5.5]undecane (CAS 3970-87-4) in 5.6 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 58 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1-oxa-4-azaspiro[5.5]undec-4-yl)ethanone.

¹H-NMR (300 MHz, RT, DMSO-d6, selected signals): δ=1.21-1.33 (m, 1H); 1.46-1.65 (m, 4H); 1.73-1.85 (m+s, 4H); 1.89-2.04 (m, 2H); 3.35-3.70 (m, s, 9H); 3.88 (dd, 1H); 4.05 (m); 4.63 (q, 1H); 7.42-7.52 (m, 5H).

Optical rotation: [α_{D}]=−39.1° (methanol, c=1 g/100 ml).

### Example 10

### (−)-1-(3-Azabicyclo[3.2.1]oct-3-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone

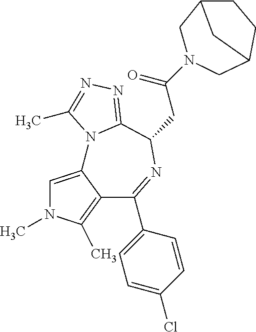 [SMILES: Cc1c2c(cn1C)-n1c(C)nnc1[C@H](CC(=O)N1CC3CCC(C3)C1)N=C2c1ccc(Cl)cc1]

A solution of 400 mg of Intermediate 6A, 0.58 ml of triethylamine, 594 mg of HATU and 169 mg of 3-azabicyclo[3.2.1]octane hydrochloride (CAS 279-82-3) in 5.6 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 170 mg of (−)-1-(3-azabicyclo[3.2.1]oct-3-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone.

¹H-NMR (300 MHz, RT, DMSO-d6, selected signals): δ=1.21-1.38 (m, 1H); 1.48-1.70 (m, 5H); 1.81 (s, 3H); 2.23 (bd, 2H); 2.61 (t, 1H); 3.13-3.27 (m, 1.5H); 3.66 (s, 3H); 3.84-3.93 (m, 1H); 4.09 (dt, 1H); 4.66 (q, 1H); 7.43-7.53 (m, 5H).

Optical rotation: [α_{D}]=−55.4° (methanol, c=1 g/100 ml).

### Example 11

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(7-oxa-2-azaspiro[3.5]non-2-yl)ethanone

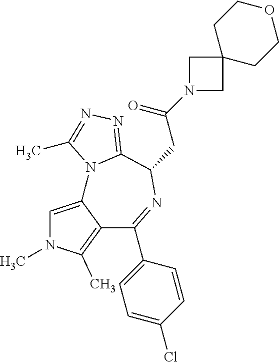 [SMILES: Cc1c2c(cn1C)-n1c(C)nnc1[C@H](CC(=O)N1CC3(CCOCC3)C1)N=C2c1ccc(Cl)cc1]

A solution of 380 mg of Intermediate 6A, 0.55 ml of triethylamine, 564 mg of HATU and 235 mg of 7-oxa-2-azaspiro[3.5]nonane oxalate (CAS 194157-10-3) in 5.4 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 120 mg of (−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(7-oxa-2-azaspiro[3.5]non-2-yl)ethanone.

¹H-NMR (300 MHz, RT, DMSO-d6, selected signals): δ=1.67-1.77 (m, 4H); 1.80 (s, 3H); 3.05 (dd, 1H); 3.20 (dd, 1H); 3.49-3.58 (m, 4H); 3.61 (d, 2H); 3.65 (s, 3H); 4.03 (d, 1H); 4.16 (d, 1H); 4.54 (t, 1H); 7.44 (s, 1H); 7.47 (s, 4H).

Optical rotation: [α_{D}]=−37.3° (methanol, c=1 g/100 ml).

### Example 12

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(2-oxa-7-azaspiro[3.5]non-7-yl)ethanone

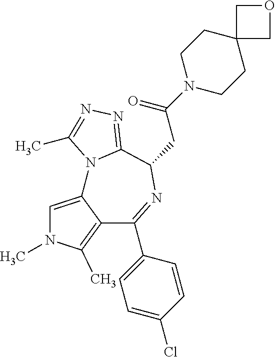 [SMILES: Cc1c2c(cn1C)-n1c(C)nnc1[C@H](CC(=O)N1CCC3(CC1)COC3)N=C2c1ccc(Cl)cc1]

A solution of 300 mg of Intermediate 6A, 0.44 ml of triethylamine, 445 mg of HATU and 109 mg of 2-oxa-7-azaspiro[3.5]nonane (CAS 241820-91-7) in 4.2 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 113 mg of (−) 2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-7-azaspiro[3.5]non-7-yl)ethanone.

¹H-NMR (300 MHz, RT, DMSO-d6, selected signals): δ=1.70 (t, 1H); 1.80 (s, 3H); 1.84-1.93 (m, 1H); 3.23-3.44 (m+water), 3.50-3.61 (m, 3H); 3.66 (s, 3H); 4.30-4.40 (m, 3H); 4.64 (t, 1H); 7.41-7.51 (m, 5H).

Optical rotation: [α_{D}]=−29.4° (methanol, c=1 g/100 ml).

### Example 13

### (−)-2-[(4S)-6-(4-Chlorophenyl)-1,7,8-trimethyl-4,8-dihydropyrrolo[3,4-f][1,2,4]triazolo[4,3-a][1,4]diazepin-4-yl]-1-(2-oxa-6-azaspiro[3.4]oct-6-yl)ethanone

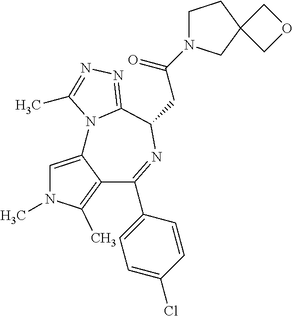 [SMILES: Cc1c2c(cn1C)-n1c(C)nnc1[C@H](CC(=O)N1CCC3(COC3)C1)N=C2c1ccc(Cl)cc1]

A solution of 300 mg of Intermediate 6A, 0.44 ml of triethylamine, 445 mg of HATU and 97 mg of 2-oxa-6-azaspiro[3.5]octane (CAS 220290-68-6) in 4.2 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 57 mg of (+2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-6-azaspiro[3.4]oct-6-yl)ethanone.

¹H-NMR (300 MHz, RT, DMSO-d6, selected signals): δ=1.80 (s, 3H); 2.09-2.17 (m, 1H); 2.21-2.29 (m, 1H); 3.17-3.49 (m+water); 3.55 (d, 1H); 3.62-3.75 (m+s, 4H); 3.95 (dd, 1H); 4.46-4.67 (m, 4H); 7.42-7.52 (m, 5H).

Optical rotation: [α_{D}]=−35.2° (methanol, c=1 g/100 ml).

### Example 14

### (−)-1-(2-Azabicyclo[2.2.2]oct-2-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone

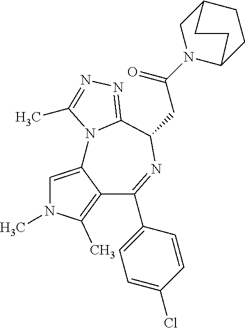 [SMILES: Cc1c2c(cn1C)-n1c(C)nnc1[C@H](CC(=O)N1CC3CCC1CC3)N=C2c1ccc(Cl)cc1]

A solution of 300 mg of Intermediate 6A, 0.44 ml of triethylamine, 445 mg of HATU and 126 mg of 2-azabicyclo[2.2.2]octane hydrochloride (CAS 5845-15-8) in 4.2 ml of DMF was stirred at RT overnight. The mixture was added to saturated sodium chloride solution/water and extracted three times with dichloromethane and the extracts were washed twice with saturated sodium chloride solution, dried over sodium sulphate and concentrated under reduced pressure. The crude product obtained was purified by chromatography on silica gel (dichloromethane/methanol gradient). This gave 150 mg of (−)-1-(2-azabicyclo[2.2.2]oct-2-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone.

¹H-NMR (300 MHz, RT, DMSO-d6, selected signals): δ=1-55-1.78 (m, 8H); 1.80 (s, 3H); 1.86-1.99 (2m, 2H); 3.18-3.43 (m+water); 3.63-3.78 (m+s, 5H); 4.68 (t, 1H); 7.43-7.53 (m, 5H).

Optical rotation: [α_{D}]=−43.3° (methanol, c=1 g/100 ml).

### Example 15

### 2-[(4S)-6-(4-Chlorophenyl)-1,8-dimethyl-4,8-dihydropyrazolo[3,4-f][1,2,4]triazolo[4,3-a][1,4]diazepin-4-yl]-1-(1,1-dioxido-1-thia-6-azaspiro[3.3]hept-6-yl)ethanone

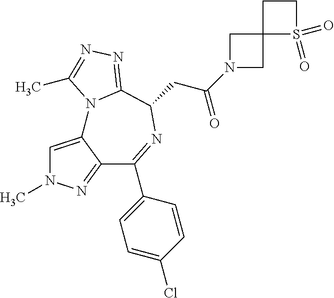 [SMILES: Cc1nnc2n1-c1cn(C)nc1C(c1ccc(Cl)cc1)=N[C@H]2CC(=O)N1CC2(CCS2(=O)=O)C1]

A solution of 290 mg of Intermediate 4F, 0.41 ml of triethylamine, 421 mg of HATU and 193 mg of Intermediate 3A in 4 ml of DMF was stirred at RT overnight. The mixture was purified by RP-HPLC chromatography (XBridge C18 5 μm 100×30 mm, mobile phase water/acetonitrile gradient, 0.1% formic acid added, flow rate 50 ml/min) This gave 162 mg of 2-[(4S)-6-(4-chlorophenyl)-1,8-dimethyl-4,8-dihydropyrazolo[3,4-f][1,2,4]triazolo[4,3-a][1,4]diazepin-4-yl]-1-(1,1-dioxido-1-thia-6-azaspiro[3.3]hept-6-yl)ethanone.

¹H-NMR (300 MHz, RT, DMSO-d6, selected signals): δ=3.18 (ddd, 1H); 3.23 (d, 1H); 4.05 (s, 3H); 4.10-4.36 (m, 4H); 4.56-4.72 (m, 2H); 4.79 (d, 1H); 7.43-7.53 (m, 2H); 7.73 (dd, 2H); 8.60 (s, 1H).

### Biological Efficacy of the Compounds According to the Invention

### 1. Assays

### 1.1 Protein-Protein Interaction Assay

### Binding Assay BRD4/Acetylated Peptide H4 (“PRO”)

To assess the BRD4 binding strength of the substances described in this application, the ability thereof to inhibit the interaction between BRD4 (BD1) and acetylated histone H4 in a dose-dependent manner was quantified.

For this purpose, a time-resolved fluorescence resonance energy transfer (TR-FRET) assay was used, which measures the binding between N-terminally His₆-tagged BRD4 (BD1) (amino acids 67-152, longer constructs also being possible, preferably amino acids 44-168) and a synthetic acetylated histone H4 (Ac-H4) peptide with sequence GRGK(Ac)GGK(Ac)GLGK(Ac)GGAK(Ac)RHGSGSK-biotin. The recombinant BRD4 protein produced in-house according to Filippakopoulos et al., Nature, 2010, 468:1119-1123 was expressed in E. coli and purified by means of (Ni-NTA) affinity and (Sephadex G-75) size exclusion chromatography. The Ac-H4 peptide can be purchased, for example, from Biosyntan (Berlin, Germany).

In the assay, typically 11 different concentrations of each substance (0.1 nM, 0.33 nM, 1.1 nM, 3.8 nM, 13 nM, 44 nM, 0.15 μM, 0.51 μM, 1.7 μM, 5.9 μM and 20 μM) were analysed as duplicates on the same microtitre plate. For this purpose, 100-fold concentrated solutions in DMSO were prepared by serial dilutions (1:3.4) of a 2 mM stock solution into a clear, 384-well microtitre plate (Greiner Bio-One, Frickenhausen, Germany). From this, 50 nl were transferred into a black test plate (Greiner Bio-One, Frickenhausen, Germany). The test was started by the addition of 2 μl of a 2.5-fold concentrated BRD4 solution (final concentration typically 10 nM in the 5 μl of reaction volume) in aqueous assay buffer [50 mM HEPES pH 7.5, 50 mM sodium chloride (NaCl), 0.25 mM CHAPS and 0.05% serum albumin (BSA)] to the substances in the test plate. This was followed by a 10-minute incubation step at 22° C. for the pre-equilibration of putative complexes between BRD4 and the substances. Subsequently, 3 μl of a 1.67-fold concentrated solution (in assay buffer) consisting of Ac-H4 peptide (83.5 nM) and TR-FRET detection reagents [16.7 nM anti-6His-XL665 and 3.34 nM streptavidin cryptate (both from Cisbio Bioassays, Codolet, France), and 668 mM potassium fluoride (KF)] were added.

The mixture was then incubated in the dark at 22° C. for one hour and then at 4° C. for at least 3 hours and for no longer than overnight. The formation of BRD4/Ac-H4 complexes was determined by the measurement of the resonance energy transfer from the streptavidin-Eu cryptate to the anti-6His-XL665 antibody present in the reaction. For this purpose, the fluorescence emission was measured at 620 nm and 665 nm after excitation at 330-350 nm in a TR-FRET measuring instrument, for example a Rubystar or Pherastar (both from BMG Lab Technologies, Offenburg, Germany) or a Viewlux (Perkin-Elmer). The ratio of the emissions at 665 nm and at 622 nm was taken as an indicator of the amount of BRD4/Ac-H4 complexes formed.

The data (ratios) obtained were normalized, with 0% inhibition corresponding to the mean from the measurements for a set of controls (typically 32 data points) in which all the reagents were present. In these, in place of test substances, 50 nl of DMSO (100%) were used Inhibition of 100% corresponded to the mean from the measurements for a set of controls (typically 32 data points) in which all the reagents except BRD4 were present. The IC₅₀ was determined by regression analysis based on a 4-parameter equation (minimum, maximum, IC₅₀, Hill; Y=max+(min−max)/(1+(X/Ic₅₀)^{Hill})).

### 1.2 Cell Assays

### Cell Proliferation Assays

In accordance with the invention, the ability of the substances to inhibit cell proliferation was determined Cell viability was determined by means of the alamarBlue® reagent (Invitrogen) in a Victor ×3 Multilabel Reader (Perkin Elmer). The excitation wavelength was 530 nm and the emission wavelength 590 nM.

The MOLM-13 cells (DSMZ, ACC 554) were sown at a concentration of 4000 cells/well in 100 μl of growth medium (RPMI1640, 10% FCS) on 96-well microtitre plates.

The B16F10 cells (ATCC, CRL-6475) were sown at a concentration of 300-500 cells/well in 100 μl of growth medium (DMEM with phenol red, 10% FCS) on 96-well microtitre plates.

The MOLP-8 cells (DSMZ, ACC 569) were sown at a concentration of 4000 cells/well in 100 μl of growth medium (RPMI1640, 20% FCS) on 96-well microtitre plates.

The LAPC-4 cells (ATCC, PTA-1441TM) were sown at a concentration of 4000 cells/well in 100 μl of growth medium (RPMI1640, 2 mM L-glutamine, 10% cFCS) on 96-well microtitre plates. One day later, the LAPC-4 cells were treated with 1 nM methyltrienolone and various substance dilutions.

The MDA-MB-231 cells (DSMZ, ACC 732) were sown at a concentration of 4000 cells/well in 100 μl of growth medium (DMEM/Ham's F12 medium, 10% FCS) on 96-well microtitre plates.

After overnight incubation at 37° C., the fluorescence values (CI values) were determined. Then the plates were treated with various substance dilutions (1E-5 M, 3E-6 M, 1E-6 M, 3E-7 M, 1E-7 M, 3E-8 M, 1E-8 M) and incubated at 37° C. over 96 (MOLM-13, B16F10, MDA-MB-431 cells), 120 (MOLP-8 cells) or 168 (LAPC-4 cells) hours. Subsequently, the fluorescence values were determined (CO values). For the data analysis, the CI values were subtracted from the CO values and the results were compared between cells which had been treated with various dilutions of the substance or only with buffer solution. The IC50 values (substance concentration needed for 50% inhibition of cell proliferation) were calculated therefrom.

The substances were tested in the cell lines in Table 1, which represent the indications specified by way of example:

TABLE 1

| Cell line | Source | Indication |
|---|---|---|
| MOLM-13 | DSMZ | acute myeloid leukaemia |
| B16F10 | ATCC | melanoma (BRAF wild-type) |
| MOLP-8 | DSMZ | multiple myeloma |
| LAPC-4 | ATCC | prostate cancer |
| MDA-MB-231 | DSMZ | mammary carcinoma |

### 2. Results:

### 2.1 Binding Assay

Table 2 shows the results from the BRD4 (BD1) binding assay.

TABLE 2

| | IC₅₀ (BRD4) |
|---|---|
| Example | (μmol/l) |
| 1 | 0.15. |
| 2 | 0.14 |
| 3 | 0.02 |
| 4 | 0.46 |
| 5 | 1.09 |
| 6 | 0.24 |
| 7 | 0.21 |
| 8 | 0.19 |
| 9 | 0.08 |
| 10 | 0.31 |
| 11 | 0.69 |
| 12 | 0.12 |
| 13 | 0.07 |
| 14 | 0.10 |
| 15 | 0.80 |

### 2.2 Cell Assays

The table shows the results from various cell proliferation assays.

TABLE 3

| | | | | | multiple |
|---|---|---|---|---|---|
| | Leukaemia | Prostate | melanoma | Breast | myeloma |
| | MOLM-13 | LAPC-4 | B16F10 | MDA-MB-231 | MOLP-8 |
| Exam- | IC₅₀ | IC₅₀ | IC₅₀ | IC₅₀ | IC₅₀ |
| ple | (μmol/l) | (μmol/l) | (μmol/l) | (μmol/l) | (μmol/l) |
| 1 | 0.71 | 0.68 | 1.14 | | |
| 2 | 0.90 | 0.92 | 2.01 | | |
| 3 | 0.11 | 0.11 | 0.16 | 0.30 | 0.14 |
| 6 | 1.24 | 0.52 | 0.89 | | |
| 8 | 0.75 | 0.54 | | | |
| 9 | 1.05 | 1.07 | | | |
| 10 | 1.46 | | | | |
| 12 | 0.48 | | | | |
| 13 | 0.41 | 0.23 | | | |
| 14 | 0.69 | 0.52 | | | |
| 15 | 2.70 | | | | |

### 3. Determination of Stability in Human Plasma

Isolated human liver microsomes (HLM) were used to assess the metabolic stability of compounds of general formula I.

The incubations were conducted with 2.4 ml of HLM solution (protein content 0.5 mg/ml), 30 μl of the test compound (final concentration 1 μM) and 0.6 ml of the cofactor mixture (=NADPH-generating system composed of 3 IU glucose-6-phosphate dehydrogenase, 14.6 mg glucose-6-phosphate, 1.2 mg NADP) at 37° C. in 100 mM phosphate buffer at pH 7.4. Samples were taken at 6 time points (2-60 min) and precipitated with an equal volume of methanol, and the recovery of the test substances used in the supernatant was determined by LC-MS/MS analysis. The half-life of substance degradation determined therefrom was used to calculate what is called the intrinsic clearance of the substance in the liver microsome preparation. With the aid of this, using various physiological parameters, a (metabolic) in vivo clearance with respect to phase I reactions was predicted according to the well-stirred model.

1. A compound of formula I 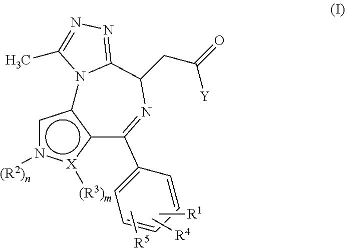
where
X represents a carbon or nitrogen atom,
n and m independently of one another represent 0 or 1,
R¹, R⁴ and R⁵ identical or different from one another represent hydrogen, hydroxy, cyano, nitro, amino, aminocarbonyl-, halogen or represent C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, aminocarbonyl-, C₁-C₆-alkylcarbonyl-, C₁-C₆-alkylaminocarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms, where the monocyclic heterocyclyl and heteroaryl radicals for their part are optionally monosubstituted by C₁-C₃-alkyl,
or
represents C₃-C₁₀-cycloalkyl- which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represents monocyclic heteroaryl- having 5 or 6 ring atoms which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylsulphonyl-, C₁-C₆-alkylamino-C₁-C₆-alkyl, halo-C₁-C₆-alkyl-, halo-C₁-C₆-alkoxy-, C₃-C₁₀-cycloalkyl- and a monocyclic heterocyclyl radical having 3 to 8 ring atoms,
or
represents monocyclic heterocyclyl- having 3 to 8 ring atoms which is optionally mono- or polysubstituted by identical or different substituents from the group consisting of halogen, amino, hydroxy, cyano, oxo, carboxy, C₁-C₆-alkyl-, C₁-C₆-alkoxy-, C₁-C₆-alkylcarbonyl-, C₁-C₆-alkoxy-C₁-C₆-alkyl-, C₁-C₆-alkylamino-, amino-C₁-C₆-alkyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
or
represents phenyl- which is optionally mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylcarbonyl-, C₁-C₆-alkylaminosulphonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
R³ represents hydrogen, hydroxy, cyano, nitro, amino, aminocarbonyl-, halogen or represents C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a carbon atom,
or
R³ represents hydrogen or represents C₁-C₆-alkyl-, C₁-C₆-alkoxy-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl-which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a nitrogen atom,
or
R² and R³ together with the ring atoms N and X may form a further heteroaromatic or heterocyclic ring having 5 to 7 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, nitro, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, pyridinyl-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
or an enantiomer, diastereomer, tautomer, solvate, physiologically acceptable salt or solvate of a salt thereof.

2. A compound according to claim 1 in which
X represents a carbon or nitrogen atom,
n and m independently of one another represent 0 or 1,
R¹, R⁴ and R⁵ identical or different from one another represent hydrogen, hydroxy, cyano, nitro, amino, aminocarbonyl-, halogen or represent C₁-C₆-alkyl-, C₁-C₆-alkoxy-, C₁-C₆-alkylamino-, C₁-C₆-alkylcarbonylamino-, C₁-C₆-alkylaminocarbonyl-, C₁-C₆-alkylcarbonyl-, C₁-C₆-alkylsulphonyl-, phenylsulphonyl- or C₁-C₆-alkylaminosulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino-, amino-C₁-C₆-alkyl-, monocyclic heterocyclyl- having 3 to 8 ring atoms and monocyclic heteroaryl- having 5 or 6 ring atoms, where the monocyclic heterocyclyl and heteroaryl radicals for their part are optionally monosubstituted by C₁-C₃-alkyl,
or
represents C₃-C₁₀-cycloalkyl- which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represent monocyclic heterocyclyl- having 3 to 8 ring atoms which is optionally mono- or polysubstituted by identical or different substituents from the group consisting of halogen, amino, hydroxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylamino-C₁-C₆-alkyl, halo-C₁-C₆-alkyl-, halo-C₁-C₆-alkoxy- and a monocyclic heterocyclyl radical having 3 to 8 ring atoms,
or
represents monocyclic heteroaryl- having 5 or 6 ring atoms which is optionally mono- or polysubstituted by identical or different substituents from the group consisting of halogen, amino, hydroxy, cyano, nitro, carboxy, C₁-C₆ alkyl-, C₁-C₆ alkoxy-, C₁-C₆ alkoxy-, C₁-C₆ alkyl-, hydroxy-C₁-C₆-alkyl-, C₁-C₆-alkylaminocarbonyl-, C₁-C₆-alkylaminosulphonyl-, C₁-C₆-alkylsulphonyl-, C₁-C₆ alkylamino-C₁-C₆ alkyl, halo-C₁-C₆-alkyl-, halo-C₁-C₆ alkoxy-, C₃-C₁₀-cycloalkyl- and a monocyclic heterocyclyl radical having 3 to 8 ring atoms,
or
represents phenyl- which is optionally mono- or polysubstituted by identical or different substituents from the group consisting of halogen, amino, hydroxy, cyano, nitro, carboxy, C1-C6-alkyl-, C1-C6-alkoxy-, C1-C6-alkoxy-C1-C6-alkyl-, C1-C6-alkylamino-, amino-C1-C6-alkyl-, C1-C6-alkylaminocarbonyl-, C1-C6-alkylaminosulphonyl-, C1-C6-alkylsulphonyl-, C1-C6-alkylamino-C1-C6-alkyl-, hydroxy-C1-C6-alkyl-, halo-C1-C6-alkyl-, halo-C1-C6-alkoxy-, C3-C10-cycloalkyl- and a monocyclic heterocyclyl radical having 3 to 8 ring atoms,
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆ alkoxy-, C₁-C₆ alkoxy-C₁-C₆ alkyl-, C₁-C₆-alkylamino- and amino-C₁-C₆-alkyl-, if X represents a carbon atom,
or
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆ alkoxy C₁-C₆ alkoxy C₁-C₆ alkyl C₁-C₆-alkylamino- and amino-C₁-C₆-alkyl-, if X represents a nitrogen atom,
or
R² and R³ together with the ring atoms N and X may form a further heteroaromatic or heterocyclic ring having 5 to 7 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, hydroxy, amino, oxo, carboxy, C₁-C₆-alkyl-, C₁-C₆-alkoxy-, C₁-C₆-alkoxy-C₁-C₆-alkyl-, hydroxy-C₁-C₆-alkyl-, C₁-C₆-alkylamino-, amino-C₁-C₆-alkyl-, C₁-C₆-alkylamino-C₁-C₆-alkyl-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
or an enantiomer, diastereomer, tautomer, solvate, physiologically acceptable salt or solvate of a salt thereof.

3. A compound according to claim 1 in which
X represents a carbon or nitrogen atom,
n and m independently of one another represent 0 or 1,
R¹, R⁴ and R⁵ identical or different from one another represent hydrogen, hydroxy, cyano, aminocarbonyl-, halogen or represent C₁-C₆-alkyl-, C₁-C₆-alkoxy, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-,
R² represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy and carboxy,
or
represents C₃-C₁₀-cycloalkyl- which is optionally mono- or polysubstituted by identical or different substituents from the group consisting of halogen, C₁-C₆-alkyl- and C₁-C₆-alkoxy-,
or
represents phenyl- which is optionally mono- or polysubstituted by identical or different substituents from the group consisting of halogen, hydroxy, cyano, carboxy, C₁-C₆-alkyl-, C₁-C₆-alkoxy-, halo-C₁-C₆-alkyl-, halo-C₁-C₆-alkoxy-, C₃-C₁₀-cycloalkyl- and a monocyclic heterocyclyl radical having 3 to 8 ring atoms,
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a carbon atom,
or
R³ represents hydrogen or represents C₁-C₆-alkyl-, C₁-C₆-alkylcarbonyl-, phenylsulphonyl- or C₁-C₆-alkylsulphonyl- which are optionally mono- or polysubstituted by identical or different substituents from the group consisting of halogen, amino, hydroxy, carboxy, hydroxy-C₁-C₆-alkyl-, C₁-C₆-alkoxy-, C₁-C₆-alkoxy-C₁-C₆-alkyl-, C₁-C₆-alkylamino- and amino-C₁-C₆-alkyl-, if X represents a nitrogen atom,
or
R² and R³ together with the ring atoms N and X may form a further heteroaromatic or heterocyclic ring having 5 to 7 ring atoms which may optionally be mono- or polysubstituted by identical or different substituents from the group consisting of halogen, amino, hydroxy, cyano, nitro, carboxy, C₁-C₆-alkyl-, C₁-C₆-alkoxy-, C₁-C₆-alkoxy-C₁-C₆-alkyl-, C₁-C₆-alkylamino-, amino-C₁-C₆-alkyl-, C₁-C₆-alkylaminocarbonyl-, C₁-C₆-alkylaminosulphonyl-, C₁-C₆-alkylcarbonyl-, C₁-C₆-alkylsulphonyl-, C₁-C₆-alkylamino-C₁-C₆-alkyl-, hydroxy-C₁-C₆-alkyl-, halo-C₁-C₆-alkyl-, halo-C₁-C₆-alkoxy-, C₃-C₁₀-cycloalkyl- and a monocyclic heterocyclyl radical having 3 to 8 ring atoms,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, hydroxy, amino, oxo, C₁-C₆-alkyl-, C₁-C₆-alkoxy-, halo-C₁-C₆-alkyl-, halo-C₁-C₆-alkoxy-, C₃-C₁₀-cycloalkyl-, phenyl-, halophenyl-, phenyl-C₁-C₆-alkyl-, phenyl-C₁-C₆-alkoxy-, —C(═O)—NR⁶R⁷, —C(═O)—R⁸, —S(═O)₂—NR⁶R⁷, —S(═O)—R⁹, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₆-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-, and
R⁹ represents C₁-C₆-alkyl-,
or an enantiomer, diastereomer, tautomer, solvate, physiologically acceptable salt or solvate of a salt thereof.

4. A compound according to claim 1 in which
X represents a carbon or nitrogen atom,
n and m independently of one another represent 0 or 1,
R¹, R⁴ and R⁵ identical or different from one another represent hydrogen, cyano, halogen or represent C₁-C₆-alkyl- or C₁-C₆-alkoxy- which is optionally mono- or polysubstituted by halogen,
R² represents hydrogen or represents C₁-C₆-alkyl- which is optionally mono- or polysubstituted by halogen,
or
represent phenyl which is optionally mono- or polysubstituted by identical or different substituents from the group consisting of halogen, cyano, C₁-C₆ alkyl-, C₁-C₆ alkoxy-, halo-C₁-C₆-alkyl- and halo-C₁-C₆-alkoxy-,
R³ represents hydrogen or C₁-C₆-alkyl- which is optionally mono- or polysubstituted by identical or different substituents from the group consisting of halogen, amino, hydroxy, C₁-C₆-alkoxy- and C₁-C₆-alkoxy-C₁-C₆-alkyl-, if X represents a carbon atom,
or
R³ represents hydrogen or C₁-C₆-alkyl- which is optionally mono- or polysubstituted by identical or different substituents from the group consisting of halogen and C₁-C₆-alkoxy-, if X represents a nitrogen atom,
or
R² and R³ together with the ring atoms N and X may form a further heteroaromatic or heterocyclic ring having 5 to 7 ring atoms which is optionally mono- or polysubstituted by identical or different substituents from the group consisting of halogen, amino, hydroxy, cyano, C₁-C₆-alkyl-, C₁-C₆-alkoxy-, halo-C₁-C₆-alkyl- and halo-C₁-C₆-alkoxy-,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, cyano, oxo, hydroxy, C₁-C₃-alkyl-, C₁-C₃-alkoxy-, halo-C₁-C₃-alkyl-, halo-C₁-C₃-alkoxy-, C₃-C₇-cycloalkyl-, phenyl-, halophenyl-, phenyl-C₁-C₃-alkyl-, phenyl-C₁-C₃-alkoxy-, —C(═O)—NR⁶R⁷, —C(═O)—R⁸, —S(═O)₂—NR⁶R⁷, —S(═O)₂—R⁹, —NH—S(═O)₂—R⁹ and a monocyclic heterocyclyl radical having 3 to 8 ring atoms,
R⁶ and R⁷ independently of one another represent hydrogen, C₁-C₃-alkyl-, cyclopropyl- or di-C₁-C₃-alkylamino-C₁-C₃-alkyl-,
R⁸ represents hydroxy, C₁-C₆-alkyl-, C₁-C₃-alkoxy-, halo-C₁-C₃-alkyl-, hydroxy-C₁-C₃-alkyl-, C₁-C₃-alkoxy-C₁-C₃-alkyl-, C₃-C₈-cycloalkyl-, phenyl-, monocyclic heterocyclyl- having 3 to 8 ring atoms or monocyclic heteroaryl- having 5 or 6 ring atoms, where phenyl-, heteroaryl- and heterocyclyl- are optionally mono- or disubstituted by halogen, C₁-C₃-alkoxy- or C₁-C₃-alkyl-,
R⁹ represents C₁-C₆-alkyl-,
or an enantiomer, diastereomer, tautomer, solvate, physiologically acceptable salt or solvate of a salt thereof.

5. A compound according to claim 1 in which
X represents a carbon or nitrogen atom,
n and m independently of one another represent 0 or 1,
R¹, R⁴ and R⁵ identical or different from one another represent hydrogen or halogen,
R² represents hydrogen or represents C₁-C₃-alkyl-,
R³ represents hydrogen or C₁-C₃-alkyl-, if X represents a carbon atom,
or
R³ represents hydrogen or represents C₁-C₃-alkyl-, if X represents a nitrogen atom,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of halogen, oxo, cyano, hydroxy, C₁-C₃-alkyl-, C₁-C₃-alkoxy-, halo-C₁-C₃-alkyl-, halo-C₁-C₃-alkoxy-, phenyl-, halophenyl-, phenyl-C₁-C₃-alkyl-, phenyl-C₁-C₃-alkoxy-, —C(═O)—NR⁶R⁷, —C(═O)—R⁸, —S(═O)₂—R⁹ and —NH—S(═O)₂—R⁹, R⁶ and R⁷ independently of one another represent hydrogen or C₁-C₃-alkyl,
R⁸ represents hydroxy, C₁-C₆-alkyl-, C₁-C₃-alkoxy-, halo-C₁-C₃-alkyl-, phenyl- or monocyclic heterocyclyl- having 3 to 8 ring atoms where phenyl- and heterocyclyl- are optionally mono- or disubstituted by halogen, C₁-C₃-alkoxy- or C₁-C₃-alkyl-,
R⁹ represents C₁-C₃-alkyl-,
or an enantiomer, diastereomer, tautomer, solvate, physiologically acceptable salt or solvate of a salt thereof.

6. A compound according to claim 1 in which
X represents a carbon or nitrogen atom,
n and m independently of one another represent 0 or 1,
R¹ represents hydrogen or halogen,
R² represents hydrogen or represents C₁-C₃-alkyl-,
R³ represents hydrogen or C₁-C₃-alkyl-, if X represents a carbon atom,
or
R³ represents hydrogen or represents C₁-C₃-alkyl-, if X represents a nitrogen atom,
R⁴ and R⁵ represent hydrogen,
Y represents a spirocycloalkyl- or heterospirocycloalkyl radical of 7 to 12 ring atoms, a bridged cycloalkyl radical or a bridged heterocycloalkyl radical of 7 to 12 ring atoms, where the radicals mentioned here are optionally mono- or polysubstituted by identical or different substituents from the group consisting of oxo, fluoro, chloro, bromo, cyano, hydroxy, methyl, ethyl, methoxy, ethoxy, trifluoromethyl, benzyl, phenyl and —C(═O)—R⁸,
R⁸ represents hydroxy, C₁-C₆-alkyl- or halo-C₁-C₃-alkyl-,
or an enantiomer, diastereomer, tautomer, solvate, physiologically acceptable salt or solvate of a salt thereof.

7. A compound according to claim 1 in which
X represents a carbon or nitrogen atom,
n and m independently of one another represent 0 or 1,
R¹ represents hydrogen, fluorine, chlorine or bromine,
R² represents hydrogen or represents methyl,
R³ represents hydrogen or methyl, if X represents a carbon atom,
or
R³ represents hydrogen or methyl, if X represents a nitrogen atom,
R⁴ and R⁵ represent hydrogen,
Y represents a spirocycloalkyl- or heterospirocycloalkyl radical of 7 to 11 ring atoms, a bridged cycloalkyl radical or a bridged heterocycloalkyl radical of 7 to 8 ring atoms, where the radicals mentioned here are optionally mono- or polysubstituted by identical or different substituents from the group consisting of oxo, fluoro, chloro, bromo, cyano, hydroxy, methyl, ethyl, methoxy, ethoxy, trifluoromethyl, benzyl, phenyl and —C(═O)—R⁸,
R⁸ represents hydroxy, C₁-C₆-alkyl- or halo-C₁-C₃-alkyl-,
or an enantiomer, diastereomer, tautomer, solvate, physiologically acceptable salt or solvate of a salt thereof.

8. A compound according to claim 1 in which
X represents a carbon or nitrogen atom,
n and m independently of one another represent 0 or 1,
R¹ represents hydrogen or chlorine,
R² represents hydrogen or represents methyl,
R³ represents methyl, if X represents a carbon atom,
or
R³ represents hydrogen or methyl, if X represents a nitrogen atom,
R⁴ and R⁵ represent hydrogen,
Y represents a radical 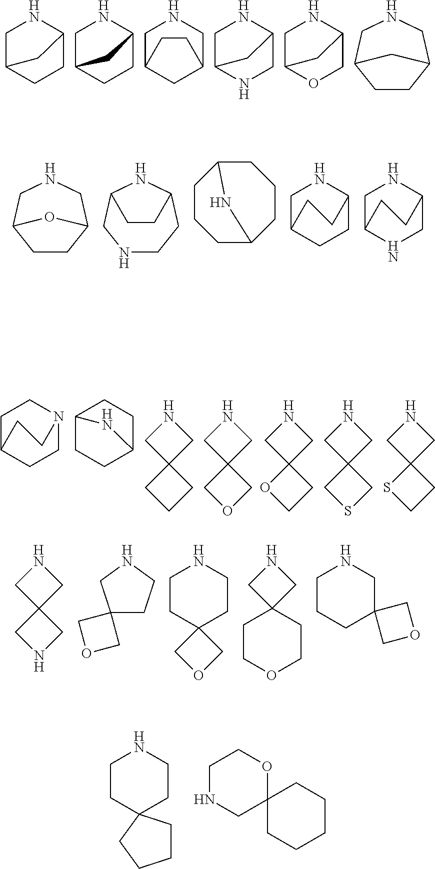
where the radicals mentioned here are optionally mono- or disubstituted by identical or different substituents from the group consisting of oxo, fluorine, chlorine, bromine, cyano, hydroxy, methyl, ethyl, methoxy, ethoxy, benzyl, phenyl and —C(═O)—R⁸,
R⁸ represents hydroxy, C₁-C₆-alkyl- or halo-C₁-C₃-alkyl-,
or an enantiomer, diastereomer, tautomer, solvate, physiologically acceptable salt or solvate of a salt thereof.

9. A compound according to claim 1 in which
X represents a carbon or nitrogen atom,
n and m independently of one another represent 0 or 1,
R¹ represents hydrogen or chlorine,
R² represents hydrogen or represents methyl,
R³ represents methyl, if X represents a carbon atom,
or
R³ represents hydrogen or methyl, if X represents a nitrogen atom,
R⁴ and R⁵ represent hydrogen,
Y represents a radical 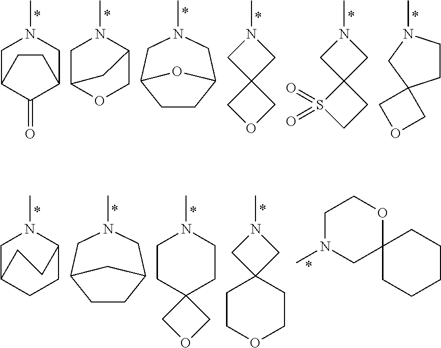
where “*” denotes the point of attachment to the remainder of the molecule,
or an enantiomer, diastereomer, tautomer, solvate, physiologically acceptable salt or solvate of a salt thereof.

10. A compound according to claim 1 selected from the group consisting of
(−)-2-[(4S)-6-(4-chlorophenyl)-1,7,8-trimethyl-4,8-dihydropyrrolo[3,4-f][1,2,4]triazolo-[4,3-a][1,4]diazepin-4-yl]-1-(2-oxa-6-azaspiro[3.3]hept-6-yl)ethan-1-one;
2-(1,7,8-trimethyl-6-phenyl-4,8-dihydropyrrolo[3,4-f][1,2,4]triazolo[4,3-a][1,4]diazepin-4-yl)-1-(2-oxa-6-azaspiro[3.3]hept-6-yl)ethan-1-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1,1-dioxo-1λ6-1-thia-6-azaspiro[3.3]hept-6-yl)ethan-1-one;
2-[(4S)-6-(4-chlorophenyl)-1,8-dimethyl-4,8-dihydropyrazolo[3,4-f][1,2,4]triazolo[4,3-a][1,4]diazepin-4-yl]-1-(2-oxa-6-azaspiro[3.3]hept-6-yl)ethan-1-one;
2-[6-(4-chlorophenyl)-1,7-dimethyl-4,7-dihydropyrazolo[3,4-f][1,2,4]triazolo[4,3-a][1,4]diazepin-4-yl]-1-(2-oxa-6-azaspiro[3.3]hept-6-yl)ethan-1-one;
(−)-8-{2-[(4S)-6-(4-chlorophenyl)-1,7,8-trimethyl-4,8-dihydropyrrolo[3,4-f][1,2,4]triazolo[4,3-a][1,4]diazepin-4-yl]acetyl}-8-azabicyclo[3.2.1]octan-3-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-{(1S,4S)-2-oxa-5-azabicyclo[2.2.1]hept-5-yl}-ethan-1-one;
(−)-2-[(4S)-6-(4-chlorophenyl)-1,7,8-trimethyl-4,8-dihydropyrrolo[3,4-f][1,2,4]triazolo[4,3-a][1,4]diazepin-4-yl]-1-(8-oxa-3-azabicyclo[3.2.1]oct-3-yl)ethan-1-one;
2-[(4S)-6-(4-chlorophenyl)-1,7,8-trimethyl-4,8-dihydropyrrolo[3,4-f][1,2,4]triazolo[4,3-a][1,4]diazepin-4-yl]-1-(1-oxa-4-azaspiro[5.5]undec-4-yl)ethanone;
1-(3-azabicyclo[3.2.1]oct-3-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone;
2-[(4S)-6-(4-chlorophenyl)-1,7,8-trimethyl-4,8-dihydropyrrolo[3,4-f][1,2,4]triazolo[4,3-a][1,4]diazepin-4-yl]-1-(7-oxa-2-azaspiro[3.5]non-2-yl)ethanone;
2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-7-azaspiro[3.5]non-7-yl)ethanone;
2-[(4S)-6-(4-chlorophenyl)-1,7,8-trimethyl-4,8-dihydropyrrolo[3,4-f][1,2,4]triazolo[4,3-a][1,4]diazepin-4-yl]-1-(2-oxa-6-azaspiro[3.4]oct-6-yl)ethanone;
1-(2-azabicyclo[2.2.2]oct-2-yl)-2-[(4S)-6-(4-chlorophenyl)-1,7,8-trimethyl-4,8-dihydropyrrolo[3,4-f][1,2,4]triazolo[4,3-a][1,4]diazepin-4-yl]ethanone;
and
2-[6-(4-chlorophenyl)-1,8-dimethyl-4,8-dihydropyrazolo[3,4-f][1,2,4]triazolo[4,3-a][1,4]diazepin-4-yl]-1-(1,1-dioxido-1-thia-6-azaspiro[3.3]hept-6-yl)ethanone.

11. A method for the treatment of a hyperproliferative disorder, benign hyperplasia, inflammatory disorder, autoimmune disorder, sepsis, viral infection, vascular disorder, atherosclerotic disorder or neurodegenerative disorder comprising administering to a patient in need thereof a therapeutically effective amount of a compound according to claim 1.

12. A method for the treatment of a tumour disorder comprising administering to a patient in need thereof a therapeutically effective amount of a compound according to claim 1.

13. A method for controlling male fertility comprising administering to a patient in need thereof an effective amount of a compound according to claim 1.

14. A method for the treatment of a leukaemia, prostate carcinoma, mammary carcinoma, melanoma or multiple myeloma comprising administering to a patient in need thereof a therapeutically effective amount of a compound according to claim 1.

15. (canceled)

16. (canceled)

17. A pharmaceutical composition comprising a compound according to claim 1 in combination with a further active compound.

18. A pharmaceutical formulation comprising a compound according to claim 1 and a pharmaceutically acceptable excipient.

19. An intermediate compound of formulae Ia or Ib 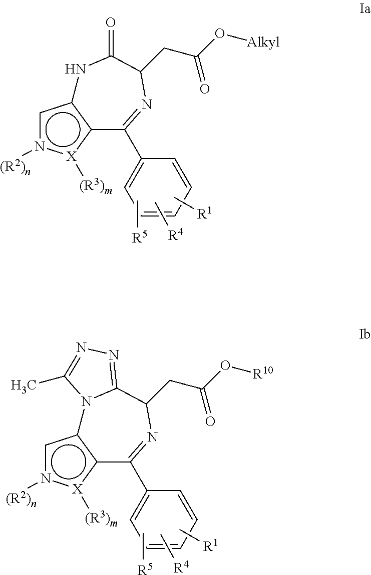
in which R¹, R², R³, R⁴, R⁵, X, n and m are defined in claim 1 and R¹⁰ represents hydrogen, or a sodium, potassium, lithium or caesium salt thereof.

20. An intermediate compound according to claim 19, selected from the group consisting of
methyl 2-(4S)-[6-(4-chlorophenyl)-1,7,8-trimethyl-4,8-dihydropyrrolo[3,4-f][1,2,4]triazolo[4,3-a][1,4]diazepin-4-yl]acetate;
methyl 2-(4S)-(1,7,8-trimethyl-6-phenyl-4,8-dihydropyrrolo[3,4-f][1,2,4]triazolo[4,3-a][1,4]diazepin-4-yl)acetate;
methyl 2-[(4S)-6-(4-chlorophenyl)-1,8-dimethyl-4,8-dihydropyrazolo[3,4-f][1,2,4]triazolo[4,3-a][1,4]diazepin-4-yl]acetate;
2-[(4S)-6-(4-chlorophenyl)-1,8-dimethyl-4,8-dihydropyrazolo[3,4-f][1,2,4]triazolo[4,3-a][1,4]diazepin-4-yl]acetic acid, sodium salt;
methyl 2-[(4S)-6-(4-chlorophenyl)-1,7-dimethyl-4,7-dihydropyrazolo[3,4-f][1,2,4]triazolo[4,3-a][1,4]diazepin-4-yl]acetate;
2-[(4S)-6-(4-chlorophenyl)-1,7-dimethyl-4,7-dihydropyrazolo[3,4-f][1,2,4]triazolo[4,3-a][1,4]diazepin-4-yl]acetic acid, sodium salt;
and
2-[(4S)-6-(4-chlorophenyl)-1,7,8-trimethyl-4,8-dihydropyrrolo[3,4-f][1,2,4]triazolo[4,3-a][1,4]diazepin-4-yl]acetate, sodium salt.
